(19) United States
(12) Patent Application Publication
(10) Pub. No.: US 2018/0313836 A1
(43) Pub. Date: Nov. 1, 2018

(54) BINDING ACTIVITY OF AMINOACYL-tRNA SYNTHETASE IN CHARCOT-MARIE-TOOTH (CMT) NEUROPATHY AND CMT-RELATED NEUROLOGICAL DISEASES

(71) Applicant: The Scripps Research Institute, La Jolla, CA (US)

(72) Inventors: Xiang-Lei Yang, San Diego, CA (US); Na Wei, San Diego, CA (US); Huihao Zhou, San Diego, CA (US); Paul Schimmel, Hobe Sound, FL (US)

(73) Assignee: The Scripps Research Institute, La Jolla, CA (US)

(21) Appl. No.: 15/767,591

(22) PCT Filed: Oct. 13, 2016
(86) PCT No.: PCT/US16/56902

§ 371 Date: Apr. 11, 2018

Related U.S. Application Data
(60) Provisional application No. 62/241,893, filed on Oct. 15, 2015.

(51) Int. Cl. G01N 33/573; G01N 33/58; G01N 33/68

(52) U.S. Cl. CPC G01N 33/573; G01N 33/581; G01N 2800/28; C12Y 601/01; G01N 33/6896

(57) ABSTRACT

Methods, compositions and kits for detecting mutated aminoacyl tRNA synthetase (aaRS) in biological samples from a subject suspect of having or suffering from a Charcot-Marie-Tooth (CMT) disease or a CMT-related disease are disclosed herein. In some embodiments, the methods include determining the amount of mutated aaRS bound to Neuropilin 1 (Nrp1). In some embodiments, methods include detection of endogenous vascular endothelial growth factor (VEGF) bound to Nrp1. Also disclosed are methods, compositions and kits for the diagnosis of a CMT or a CMT-related disease through the detection of VEGF and/or mutated aaRS in a subject.

### CROSS-REFERENCE TO RELATED APPLICATIONS

The present application claims the benefit of priority to U.S. Provisional Patent Application No. 62/241,893, filed on Oct. 15, 2015, the disclosure of which is incorporated by reference herein in its entirety.

### STATEMENT REGARDING FEDERALLY SPONSORED R&D

This invention was made with government support under R01GM088278 awarded by the U.S. National Institutes of Health. The government has certain rights in the invention.

### REFERENCE TO SEQUENCE LISTING

The material in the accompanying sequence listing is hereby incorporated by reference into this application. The accompanying sequence listing text file, named SeqListing.txt, was created on Oct. 13, 2016 and is 269 KB. The content of the sequence listing is hereby incorporated by reference in its entirety.

### BACKGROUND

### Field

The present disclosure relates generally to the field of aminoacyl-tRNA synthetases, and diagnostics and treatments of hereditary peripheral neuropathies.

### Description of the Related Art

Aminoacyl-tRNA synthetases (aaRSs) are essential housekeeping proteins that catalyze the aminoacylation of tRNA molecules as part of the decoding of genetic information during the process of protein translation. Each of the eukaryotic tRNA synthetases consists of a core enzyme, which is closely related to the prokaryotic counterpart of the tRNA synthetase, and an additional domain that is appended to the amino-terminal or carboxyl-terminal end of the core enzyme.

Glycyl tRNA synthetase (GlyRS) is a class II tRNA synthetase, whose catalytic domain consists of a central anti-parallel β sheet flanked with a helices, and three conserved sequence motifs (Xie et al., Proc. Natl. Acad. Sci. 104: 9976-9981 (2007)). GlyRS is a ubiquitously expressed enzyme in multi-cellular organisms. Like several other tRNA synthetase family members, GlyRS has acquired the ability to be secreted from cells and can influence cell signaling. Mutations in the GlyRS gene, GARS, are associated with hereditary peripheral neuropathies, such as Charcot-Marie-Tooth (CMT) diseases (He et al., Proc. Natl. Acad. Sci. 108: 12307-12312 (2011)). These mutations show loss-of-function features, suggesting that tRNA-charging deficits play a role in disease pathogenesis. Despite the broad requirement of GlyRS for protein biosynthesis in all cells, mutations in GARS cause a selective degeneration of peripheral axons leading to deficits in distal motor function. CMT is presently incurable, and although it is a rare disease, it is one of the commonest inherited neurological disorders, affecting 1 in 2,500 people (Krajewski et al., Brain 123: 1516-1527 (2000)). Current diagnosis of CMT involves a laborious and costly tiered genetic testing approach, which relies on nerve conduction velocity assessment, disease inheritance pattern, and population frequency (England et al., Neurol. 72: 185-192 (2009)). Furthermore, there are few, if any, effective treatments available for CMT, especially during the early stages of the disease. There is a need to provide effective diagnostics and treatment of CMT.

Selective neuronal loss is a hallmark of neurodegenerative diseases, which counter-intuitively are often caused by mutations in widely-expressed genes (Saxena et al., Neuron 71:35-48 (2011)). Charcot-Marie-Tooth (CMT) diseases are the most common hereditary peripheral neuropathies, for which there are no effective therapies (Skre et al., Clin. Genet. 6:98-118 (1974); Patzko et al., Curr. Neurol. Neurosci. Rep. 11:78-88 (2011)). CMT has more than 90 subtypes, each subtype linked to mutations in a specific gene. Different subtypes of CMT have similar clinical presentations, but likely different disease-causing mechanisms. For example one subtype of the CMT diseases—CMT2D—is caused by dominant mutations in GARS, encoding the ubiquitously expressed enzyme glycyl-tRNA synthetase (GlyRS). Despite the broad requirement of GlyRS for protein biosynthesis in all cells, mutations in this gene cause a selective degeneration of peripheral axons leading to deficits in distal motor function (Antonellis et al., Am. J. Hum. Genet. 72:1293-1299 (2011)). How mutations in GlyRS (GlyRS^{CMT2D}) are linked to motor neuron vulnerability has remained elusive. Some other CMT subtypes include DI-CMTC associated with tyrosyl-tRNA-synthetase (TyrRS), CMT2N associated with alanyl-tRNA synthetase (AlaRS), CMT2W associated with histidyl-tRNA synthetase (HisRS), CMTRIB associated with lysyl-tRNA synthetases (LysRS), and CMT2U associated with methionyl-tRNA synthetase (MetRS). Mutations in GlyRS and LysRS are also linked to CMT-related distal hereditary motor neurophy type V (dHMN-V) and hereditary neuropathy with liability to pressure palsies (HNPP), respectively. Diagnosis of the correct subtype of CMT and CMT-related diseases can be critical for developing effective treatment. So far, diagnosis of aminoacyl-tRNA synthetase linked CMT can only rely on genetic association studies, which cannot be performed effectively without a large family of patients. For small CMT and CMT-related disease families and for patients with de novo mutations, correct diagnosis of CMT and CMT-related disease subtypes remains a challenge.

### SUMMARY

The present disclosure shows that mutated aaRS acquires a neomorphic binding activity that directly antagonizes an essential signaling pathway for motor neuron survival. CMT2D mutations alter the conformation of aaRS, enabling mutated aaRS, such as GlyRS^{CMT2D} to bind the Neuropilin 1 (Nrp1) receptor. This aberrant interaction competitively interferes with the binding of the cognate ligand vascular endothelial growth factor (VEGF) to Nrp1. Genetic reduction of Nrp1 in mice worsens CMT2D symptoms, whereas enhanced expression of VEGF improves motor function. These findings link the selective pathology of CMT2D to the neomorphic binding activity of GlyRS^{CMT2D} that antagonizes the VEGF/Nrp1 interaction, and indicate the VEGF/Nrp1 signaling axis is an actionable target for treating CMT2D. Also described herein, the aberrant interaction between mutant aaRS and Nrp1 is a common feature for various CMT subtype diseases, and thus can be used to detect the presence of mutant aaRS in biological samples and/or in subjects suspected of having a CMT disease.

The present disclosure provides compositions, methods, kits, and diagnostic devices for the diagnosis of CMT diseases and CMT-related neurological diseases in a subject. Also provided herein are methods for the detection of mutated aaRS and endogenous VEGF bound to Nrp1.

Some embodiments provide methods for determining the presence of a mutated aaRS in a biological sample or a subject. In some embodiments, the methods comprise immobilizing a Nrp1 protein or a fragment thereof on a solid support, contacting a biological sample from a subject suspected of having a mutated aaRS with the immobilized Nrp1 protein under conditions that allows binding of Nrp1 protein to an aaRS to form an immobilized Nrp1-aaRS complex on the solid support, contacting the solid support with a detectably labeled molecule that specifically binds the aaRS, and detecting the amount of labeled Nrp1-aaRS complex on the solid support as indicative of the presence or absence of the mutated aaRS in the subject. In some embodiments, the detectably labeled molecule is an antibody against aaRS or a fragment thereof (anti-aaRS antibody). The anti-aaRS antibody can be, for example, a polyclonal antibody or a monoclonal antibody. The detectably labeled molecule can be, for example, isotopically or non-isotopically labeled. In some embodiments, the immobilizing step further comprises removing unbound Nrp1 protein or a fragment thereof from the solid support. In some embodiments, the methods (for example at the contacting step) further comprise washing the solid support to remove any unbound aaRS. In some embodiments, the methods (for example at the contacting step) further comprise removing unbounded detectably labeled molecule from the solid support. In some embodiments, the methods further comprises comparing the amount of labeled Nrp1-aaRS complex detected in the detecting step with a reference amount of Nrp1-aaRS complex from reference biological samples that do not have a mutated aaRS.

In some embodiments, the methods for determining the presence of a mutated aaRS in a biological sample or a subject comprise immobilizing a capture molecule on a solid support, wherein the capture molecule specifically binds to Nrp1 protein or a fragment thereof; contacting a biological sample suspected of containing a protein complex comprising Nrp1 protein and a mutated aaRS (Nrp1-aaRS complex) with the immobilized capture molecule under conditions that allows binding of Nrp1 protein to the capture molecule immobilized on the solid support; contacting the solid support with a detectably labeled molecule that specifically binds the aaRS; and detecting the amount of labeled Nrp1-aaRS complex on the solid support as indicative of the presence or absence of the mutated aaRS in the subject. In some embodiments, the detectably labeled molecule is a detectably labeled antibody against aaRS or a fragment thereof.

In some embodiments, methods for determining the presence of a mutated aaRS in a biological sample or in a subject suspected of having the mutant aaRS comprise: providing immobilizing a capture molecule on a solid support, where the capture molecule specifically binds to Nrp1 protein or a fragment thereof; contacting a biological sample with the immobilized capture protein under conditions that allow binding of Nrp1 protein to the capture molecule immobilized on the solid support, wherein the biological sample is suspected of containing a protein complex comprising Nrp1 protein and mutated aaRS (Nrp1-aaRS complex); contacting the solid support with a detectably labeled molecule that specifically binds VEGF under conditions that allow binding of the detectably labeled molecule to bind to VEGF to form a VEGF-containing complex; and detecting the amount of labeled VEGF-containing complex on the solid support as indicative of the presence or absence of a mutant GlyRS in the subject. In some embodiments, the detectably labeled molecule is a detectably labeled antibody against VEGF or a fragment thereof. In some embodiments, the methods further comprise comparing the amount of labeled VEGF-containing complex detected in the detecting step with a reference amount of VEGF-containing complex in subjects that do not have a mutated aaRS. In some embodiments, the capture molecule is an antibody against Nrp1 protein or a fragment thereof.

In the methods disclosed therein for determining the presence of a mutated aaRS in a biological sample or in a subject, the biological sample can, for example, comprises neural tissue, neural cells, neuroglia cells, peripheral blood, lymphoblastoid cells, cerebrospinal fluid, ependymal cells, muscle tissue, muscle cells, skin tissues, fibroblasts, or any combination thereof. In some embodiments, the biological sample comprises one or more neuronal cells. In some embodiments, the solid support comprises a bead, a microtiter plate, or a combination thereof. The Nrp1 protein or the fragment thereof can be, in some embodiments, a recombinant protein. The fragment of Nrp1 protein can comprise, for example, a b1 domain of the Nrp1 protein.

The methods for determining the presence of a mutated aaRS in a biological sample, in some embodiments, comprise obtaining and/or providing the biological sample from a subject having or suspected of having a mutated aaRS. In some embodiments, the methods comprise obtaining and/or providing the biological sample from a subject having or suspected of having a CMT disease and/or a CMT-related neurological disease. The mutated aaRS can be, for example, a mutated glycyl-tRNA synthetase (GlyRS), tyrosyl-tRNA synthetase (TyrRS), alanyl-tRNA synthetase (AlaRS), histidyl-tRNA synthetase (HisRS), lysyl-tRNA synthetase (LysRS), or methionyl-tRNA synthetase (MetRS). In some embodiments, the methods further comprise lysing cells in the biological sample. Lysing the cells in the biological sample can, in some embodiments, comprises dissociating endogenous Nrp1-aaRS complex in the biological sample. In some embodiments, the biological sample is obtained or derived from a subject suffering from a Charcot-Marie-Tooth (CMT) disease and/or a CMT-related neurological disease. The CMT disease can be, for example, CMT subtype CMT2D (or dHMN-V), DI-CMTC, CMT2N, CMT2W, CMTRIB (or HNPP), CMT2U or a combination there of.

In some embodiments, Nrp1 that is mobilized to the solid support comprises a b1 domain of the Nrp1 protein. In some embodiments, the aaRS is glycyl-tRNA synthetase (GlyRS), including but not limited to the GlyRS having the sequence of SEQ ID NOs: 10-25; tyrosyl-tRNA synthetase (TyrRS), including but not limited to the TyrRS having the sequence of SEQ ID NOs: 26-30; alanyl-tRNA synthetase (AlaRS), including but not limited to the AlaRS having the sequence of SEQ ID NO: 31-37, histidyl-tRNA synthetase (HisRS), including but not limited to the HisRS having the sequence of SEQ ID NO: 38-43; lysyl-tRNA synthetase (LysRS), including but not limited to the LysRS having the sequence of SEQ ID NO: 44-48; or methionyl-tRNA synthetase (MetRS), including but not limited to the MetRS having the sequence of SEQ ID NO: 49-51. In some embodiments, the detectable labeled molecule is an antibody against aaRS or a fragment thereof (anti-aaRS antibody). In some embodiments, the anti-aaRS antibody is a polyclonal antibody or a monoclonal antibody. In some embodiments, the detectable labeled molecule is isotopically or non-isotopically labeled.

In some embodiments, the solid support comprises a bead, a microtiter plate, or a combination thereof. In some embodiments, the Nrp1 protein is a recombinant Nrp1 protein. In some embodiments, unbound Nrp1 protein or a fragment thereof is removed from the solid support. In some embodiments, the solid support is washed to remove any unbound aaRS, and unbound detectably labeled molecule is removed from the solid support. In some embodiments, the amount of labeled Nrp1-aaRS complex detected is compared with a reference amount of Nrp1-aaRS complex in subjects that do not have a mutated aaRS.

In some embodiments, the amount of labeled VEGF-containing complex detected is compared with a reference amount of VEGF-containing complex in subjects that do not have a mutated aaRS.

Some embodiments provide methods for diagnosing a CMT disease or a CMT-related neurological disease in a subject. In some embodiments, the methods comprise isolating protein complexes comprising Nrp1 from a biological sample from a subject suspected of having a CMT disease and/or a CMT-related neurological disease, determining the amount of VEGF in the protein complexes isolated in step, and comparing the amount of VEGF with a reference amount of VEGF in subjects that do not have CMT diseases and CMT-related neurological diseases, whereby a lower VEGF amount indicates that the subject suffers from a CMT disease and/or a CMT-related neurological disease. The methods can, in some embodiments, further comprise obtaining and/or providing the biological sample from the subject.

In some embodiments, determining the amount of VEGF in the protein complexes comprises detecting VEGF using an antibody against VEGF (anti-VEGF antibody). The anti-VEGF antibody can be, for example, a polyclonal antibody or a monoclonal antibody. In some embodiments, determining the amount of VEGF in the protein complexes comprises dissociating the protein complexes.

In some embodiments, the methods further comprise determining the amount of one or more aaRS (e.g., mutant aaRS) in the isolated Nrp1 protein complexes. In some embodiments, the one or more mutated aaRS comprises a mutated GlyRS, TyrRS, AlaRS, HisRS, LysRS, MetRS, or a combination thereof. In some embodiments, at least one of the one or more aaRS is GlyRS. In some embodiments, the methods further comprise comparing the amount of at least one of the one or more aaRS determined with a reference amount of the aaRS in subjects that do not have CMT diseases and/or CMT-related neurological diseases.

In some embodiments, methods for diagnosing a CMT disease and/or a CMT-related neurological disease in a subject comprise: isolating protein complexes comprising Nrp1 from a biological sample from a subject suspected of having a CMT disease and/or a CMT-related neurological disease, determining the amount of one or more aaRS in the isolated protein complexes; and comparing the amount of the aaRS with a reference amount of aaRS in subjects that do not have CMT diseases, whereby higher aaRS amount indicates that the subject suffers from a CMT disease.

The biological sample used in the methods can, for example, comprise neural tissue, neuroglia cells, neural cells, peripheral blood, lymphoblastoid cells, cerebrospinal fluid, ependymal cells, muscle tissue, muscle cells, skin tissues, fibroblasts, or any combination thereof. In some embodiments, the methods further comprise lysing cells in the biological sample. The methods can, in some embodiments, further comprise obtaining and/or providing the biological sample from the subject.

In some embodiments, the step of isolating protein complexes comprises Nrp1 comprises immunoprecipitating the protein complexes using an antibody against Nrp1 (anti-Nrp1 antibody). The anti-Nrp1 antibody can be, for example, a polyclonal or a monoclonal antibody against Nrp1. In some embodiments, the anti-Nrp1 antibody binds to the b1 domain of Nrp1. In some embodiments, the anti-Nrp1 antibody does not bind to the b1 domain of Nrp1. In some embodiments, the anti-Nrp1 antibody binds to the intracellular domain of Nrp1.

In some embodiments, the step of determining the amount of aaRS in the protein complexes comprises detecting aaRS using an antibody against aaRS or a fragment thereof (anti-aaRS antibody). In some embodiments, the one of the one or more aaRS is GlyRS, TyrRS, AlaRS, HisRS, LysRS, or MetRS. In some embodiments, at least one of the one or more aaRS is GlyRS. The anti-aaRS antibody can be, for example, a polyclonal or monoclonal antibody. In some embodiments, the determining the amount of aaRS in the protein complexes comprises dissociating the protein complexes.

In some embodiments, the methods further comprise determining the amount of vascular endothelial growth factor (VEGF) in the protein complexes. In some embodiments, the amount of VEGF determined is compared with a reference amount of VEGF in subjects that do not have CMT diseases and/or CMT-related neurological diseases.

Some embodiments provide a method of determining the presence of a mutated aaRS in a biological sample, wherein the method comprises: isolating protein complexes comprising Nrp1 from a biological sample from a subject suspected of having a CMT disease and/or a CMT-related neurological disease; determining the amount of an aaRS in the protein complexes; and comparing the amount of aaRS with a reference amount of aaRS in subjects that do not have CMT diseases and/or CMT-related neurological diseases, whereby a higher aaRS amount in the test sample is indicative of the presence of mutated aaRS in the biological sample. In some embodiments, the aaRS is GlyRS, TyrRS, AlaRS, HisRS, LysRS, or MetRS.

In some embodiments, the mutated aaRS is a missense mutant. In some embodiments, the aaRS is GlyRS. In some embodiments, the GlyRS mutant comprises at least one amino acid substitution selected from the group consisting of A57V, E71G, P234KY, L129P, D146N, C157R, S211F, L218Q, G240R, P244L, E279D, I280F, H418R, D500N, G526R, S581L, G598A, and a combination thereof.

In some embodiments, the aaRS is TyrRS. In some embodiments, the TyrRS mutant comprises a 4 amino acid deletion of VKQV at positions 153-156, at least one amino acid substitution selected from the group consisting of G41R, D81I, E196K, or a combination thereof.

In some embodiments the aaRS is AlaRS. In some embodiments, the AlaRS mutant comprises at least one amino acid substitution selected from the group consisting of N71Y, G102R, R329H, E688G, E778A, D893N, and a combination thereof.

In some embodiments, the aaRS is HisRS. In some embodiments, the HisRS mutant comprises at least one amino acid substitution selected from the group consisting of T132I, P134H, R137Q, D175E, D364Y, and a combination thereof.

In some embodiments, the aaRS is MetRS. In some embodiments, the MetRS mutant comprises at least one amino acid substitution selected from the group consisting of R618C, P800T and a combination thereof.

In some embodiments, the aaRS is LysRS. In some embodiments, the LysRS mutant comprises at least one amino acid substitution selected from the group consisting of L133H, Y173SerfsX7, I302M, T623S, and a combination thereof.

In some embodiments, the isolated protein complexes comprising Nrp1 comprises immunoprecipitating the biological sample with an anti-NRP1 antibody.

Some embodiments provide a method comprising: providing a biological sample from a subject, isolating protein complexes comprising Neuropilin 1 (Nrp1) from the biological sample; and determining the amount of VEGF in the isolated protein complexes. In some embodiments, the biological sample comprises neural tissue, neuroglia cells, neural cells, peripheral blood, lymphoblastoid cells, cerebrospinal fluid, ependymal cells, muscle tissue, muscle cells, skin tissues, fibroblasts, or any combination thereof. In some embodiments, the method further comprises lysing cells in the biological sample. In some embodiments, and isolating protein complexes comprising Nrp1 comprises immunoprecipitating the protein complexes using an antibody against Nrp1 (anti-Nrp1 antibody). In some embodiments, the anti-Nrp1 antibody binds to the b1 domain of Nrp1. In some embodiments, the anti-Nrp1 antibody does not bind to the b1 domain of Nrp1. In some embodiments, the anti-Nrp1 antibody binds to the intracellular domain of Nrp1.

In some embodiments, determining the amount of VEGF in the protein complexes comprises detecting VEGF using an antibody against VEGF or a fragment thereof (anti-VEGF antibody). In some embodiments, determining the amount of VEGF in the protein complexes comprises dissociating the protein complexes. In some embodiments, the method further comprises determining the amount of at least one aaRS in the isolated protein complexes. In some embodiments, the method further comprises comparing the amount of the at least one aaRS from the subject with a reference amount of the at least one aaRS in subjects that do not have CMT diseases and/or CMT-related neurological diseases.

Some embodiments provide a method comprising: providing a biological sample from a subject; isolating protein complexes comprising Nrp1 from the biological sample; and determining the amount of an aaRS in the isolated protein complexes.

Some embodiments provided a method for ascertaining the presence of a mutant aaRS in a subject, comprising: providing a biological sample from a subject; isolating protein complexes comprising Nrp1 from the biological sample; and determining the amount of an aaRS in the isolated protein complexes as indicative of the presence or absence of a mutant aaRS in the subject. In some embodiments, the biological sample comprises neural tissue, neural cells, neuroglia cells, peripheral blood, lymphoblastoid cells, cerebrospinal fluid, ependymal cells, muscle tissue, muscle cells, skin tissues, fibroblasts, or any combination thereof. In some embodiments, the method further comprises lysing cells in the biological sample. In some embodiments, isolating the protein complexes comprising Nrp1 comprises immunoprecipitating the protein complexes using an antibody against Nrp1 (anti-Nrp1 antibody). In some embodiments, the anti-Nrp1 antibody binds to the b1 domain of Nrp1. In some embodiments, the anti-Nrp1 antibody does not bind to the b1 domain of Nrp1. In some embodiments, the anti-Nrp1 antibody binds to the intracellular domain of Nrp1. In some embodiments, determining the amount of an aaRS in the protein complexes comprises detecting the aaRS using an antibody against the aaRS or a fragment thereof (anti-aaRS antibody). In some embodiments, determining the amount of the aaRS in the protein complexes comprises dissociating the protein complexes. In some embodiments, the method further comprises determining the amount of VEGF in the isolated protein complexes. In some embodiments, the amount of VEGF from the isolated protein complexes is compared with a reference amount of VEGF in subjects that do not have CMT diseases and/or CMT-related neurological diseases.

Some embodiments provide kits for detecting a mutated aaRS in a biological sample. In some embodiments, the kits comprise: a cell lysis buffer; a solid support coated with an Nrp1 protein or a fragment thereof; and a detectably labeled molecule that specifically binds to an aaRS. In some embodiments, the Nrp1 protein is a recombinant Nrp1 protein. In some embodiments, the Nrp1 protein or the fragment thereof comprises a b1 domain of Nrp1 protein. In some embodiments, the detectably labeled molecule is an antibody against the aaRS or a fragment thereof (anti-aaRS antibody). The anti-aaRS antibody can be, for example, a polyclonal or a monoclonal antibody. The detectably labeled molecule can be, for example, isotopically or non-isotopically labeled. In some embodiments, the kits for detecting a mutated aaRS comprise: a cell lysis buffer; a solid support on which a capture molecule is immobilized, wherein the capture molecule specifically binds to Nrp1 protein or a fragment thereof; and a detectably labeled molecule that specifically binds to VEGF or a fragment thereof. In some embodiments, the aaRS is GlyRS, TyrRS, AlaRS, HisRS, LysRS, or MetRS. The solid support can, for example, comprise a bead, a microtiter plate, or a combination thereof.

Also provided are methods for treating CMT disease and/or CMT-related neurological diseases in a subject. The method includes, in some embodiments, acquiring knowledge of interaction of Nrp1 with one or more mutant aaRS in a biological sample from the subject; and administering a therapeutically effective amount of a treatment agent to the subject. In some embodiments, the treatment agent is VEGF or a fragment thereof. In some embodiments, the VEGF reduces or inhibits the binding of GlyRS to Nrp1. In some embodiments, the knowledge acquired comprises the extent to which GlyRS binds to Nrp1.

Also provided are kits and devices for the diagnosis or determination of CMT and/or CMT-related neurological diseases in a subject. In some embodiments, point-of-care (POC) diagnostic devices are used. In some embodiments, a lateral flow assay (LFA) is used to diagnose CMT and/or CMT-related neurological diseases in a subject. In some embodiments, the LFA comprises a solid phase having mobilizable anti-Nrp1 antibodies deposited thereon, wherein the anti-bodies bind to Nrp1 upon contact. In some embodiments, the antibody-Nrp1 complex migrates with the sample front to a detection region. In some embodiments, immobilized anti-VEGF antibodies are deposited at the detection region, and bind to the antibody-Nrp1 complex if VEGF is present. In some embodiments, a visually detectable signal indicates the presence of VEGF bound to Nrp1. In some embodiments, the signal is compared to a signal obtained from healthy subjects, wherein a less intense signal is indicative of less VEGF and a higher likelihood of having CMT and/or CMT-related neurological diseases. In some embodiments, the sample is lysed prior to deposition or placement on the LFA.

In some embodiments, methods of detecting a mutated aaRS in a biological sample or a patient comprise: providing a biological sample from a patient; and detecting whether a mutated aaRS is present in the biological sample by contacting the sample with Nrp1 and detecting the binding between the mutated aaRS and Nrp1 In some embodiments, said detecting binding between mutated aaRS and Nrp1 is indicative of Charcot-Marie-Tooth (CMT) disease in the patient.

In some embodiments, methods of diagnosing CMT disease and/or CMT-related neurological diseases in a patient comprise: providing a biological sample from a patient; detecting whether a mutated aaRS is present in the biological sample by contacting the sample with Nrp1 and detecting the binding between the mutated aaRS and Nrp1; and diagnosing the patient with CMT when the binding between the mutated aaRS and Nrp1 is detected.

The presence of various mutant aaRS, including but not limited to GlyRS, TyrRS, AlaRS, HisRS, LysRS, or MetRS, in a biological sample or a subject can be detected using the methods, compositions and kits disclosed herein. The methods, compositions and kits disclosed herein can also be used to detect interference to the interaction between VEGF and Nrp1 by mutant aaRS to ascertain the presence of the mutant aaRS. The detection of the mutant aaRS can be used to diagnose CMT diseases, including but not limited to CMT disease subtype CMT2D, DI-CMTC, CMT2N, CMT2W, or a combination there of. As disclosed herein, the CMT-related neurological diseases include, but are not limited to, distal hereditary motor neurophy (dHMN) and hereditary neuropathy with liability to pressure palsies (HNPP).

These features, together with other features herein further explained, will become obvious through a reading of the following description of the drawings and detailed description.

### BRIEF DESCRIPTION OF THE DRAWINGS

FIGS. 1A-1C show that dispersed CMT2D mutations consistently cause neomorphic structural opening at the dimer interface of GlyRS. FIG. 1A illustrates the distribution of 15 CMT2D-associated dominant mutations in the three domains of the cytosolic human GlyRS. The three strongest pathogenic mutations are E71G, L129P, and G240R. Two mutations identified in mice (*) are labeled with their corresponding residue numbers in the human protein. FIG. 1B shows the human GlyRS structure (monomeric subunit) viewed from the dimer interface. Consensus opened-up areas caused by 5 CMT2D mutations are highlighted in dark grey. FIG. 1C shows opened-up areas (highlighted in dark grey) by the P234KY mutation (>10% increase in deuterium incorporation relative to WT GlyRS).

FIG. 2 illustrates hydrogen-deuterium exchange analysis to compare P23₄KYGlyRS^{CMT2D} and GlyRS^{WT} in solution. A global increase (15%) in deuterium incorporation for the mutant GlyRS was observed indicating overall structural opening. The regions having significant changes (>10%) in deuterium incorporation are highlighted under the human cytosolic GlyRS sequence to indicate the % difference in changes.

FIGS. 3A-3L show that GlyRS^{CMT2D} specifically binds Nrp1 and antagonizes the VEGF-Nrp1 interaction and that CMT mutants of TyrRS, AlaRS, and HisRS also specifically bind Nrp1. FIG. 3A shows the results of an in vitro pull-down (PD) assay of GlyRS^{CMT2D} proteins by the ectodomain of Nrp1 but not TrkB. FIG. 3B shows the results of a co-immunoprecipitation (IP) assay to detect the GlyRS-Nrp1 interaction in neural tissues of wild-type (WT) and P234KY-Gars^{CMT2D} mice (CMT). FIG. 3C shows the results of a co-immunoprecipitation (IP) assay showing aberrant Nrp1 interaction, as detected in motor neuron NSC-34 cells, with almost all of the published CMT2D GlyRS mutations tested. The only exception is S581L. Interestingly, the S581L mutation was also found in healthy individuals, suggesting that the mutation may not be disease causing. FIG. 3D shows aberrant Nrp1 interaction, detected by co-immunoprecipitation in NSC-34 cells or by GST pull-down using purified proteins, with almost all of the published DI-CMTC TyrRS mutations. The only exception is D81I, which is a de novo mutation without strong genetic association to CMT. K265N, a non-CMT causing mutation was used as the negative control. FIG. 3E shows aberrant Nrp1 interaction, detected by co-immunoprecipitation in NSC-34 cells, with all of the published CMT2N AlaRS mutations tested. FIG. 3F shows aberrant Nrp1 interaction, detected by co-immunoprecipitation in NSC-34 cells, with all of the published CMT2W HisRS mutations. Y454S mutation, which is linked to Usher syndrome, a disease different from CMT, was used as a negative control. FIG. 3G shows the results of a co-immunoprecipitation assay that detected significantly more aberrant GlyRS-Nrp1 interaction in lymphocytes from CMT2D patients carrying the GlyRS L129P mutation (n=5) than from DI-CMTC patients carrying TyrRS G41R mutation (n=3) and from healthy individuals. Similarly, significantly more aberrant TyrRS-Nrp1 interaction was detected in lymphocytes from DI-CMTC patients carrying TyrRS g41R mutation (n=3) than from CMT2D patients carrying the GlyRS L129P mutation (n=5) and from healthy individuals. Moreover, within the five CMT2D patients carrying the GlyRS Li29P mutation from the same family, the strength of the aberrant GlyRS-Nrp1 interaction seems to correlate with the severity of the CMT2D symptoms. For example, patient 1066.29 has the most severe CMT2D symptoms and also the strongest GlyRS-Nrp1 interaction, while patient 1066.70 has the least severe symptoms and the weakest GlyRS-Nrp1 interaction. Therefore, aberrant tRNA synthetase-Nrp1 interaction may be used not only to determine the CMT pathogenicity of a tRNA synthetase mutation, but also as a companion diagnostic assay to select patients that are most suitable for targeting the aberrant interaction as a potential therapeutic. FIG. 3H shows the domain mapping using in vitro IP, which identifies the b1 domain of Nrp1 as the main binding site of GlyRS^{CMT2D}. FIGS. 3I-3J show the results of an in vitro PD assay showing the competition between P234KY-GlyRS^{CMT2D} and VEGF-A165 proteins for Nrp1 (b domains) binding. FIG. 3K provides a schematic representation of facial motor neuron migration in open-book preparations of WT (left half) and VEGF/Nrp1-deficient mouse hindbrains at E13.5 (right half). The center and right panels depict fluorescence labeling of facial motor neuron somata and axons by ISLMN:GFP-F on one side of E13.5 mouse hindbrain of open-book preparation. Scale bar represents 200 μm. FIG. 3L provides a schematic representation of facial motor nucleus in open-book preparations of WT (left half) and VEGF/Nrp1-deficient mouse hindbrains at E13.5 (right half). The center and right panels depict immunostaining of Isl-positive facial nucleus on one side of the E13.5 mouse hindbrain of open-book preparation. Scale bar represents 200 μm.

FIGS. 4A-4E show the characterization of the binding activity of GlyRS^{CMT2D}. FIG. 4A shows the results of an in vitro pull-down of P234KY-GlyRS^{CMT2D} proteins with the ectodomains of Nrp1, TrkB, DCC, Robo1, and Unc5C proteins. Note the much stronger binding of GlyRS^{CMT2D} with Nrp1 compared to other receptors. GlyRS was detected by immunoblot with anti-GlyRS antibody; similar amounts of input receptors were visualized by Coomassie Brilliant Blue staining. FIG. 4B shows the results of an in vitro pull-down of GlyRS^{CMT2D} proteins with the ectodomain of Nrp1. In addition to L129P and P234KY, direct binding to Nrp1 was detected for E71G and G240R GlyRS^{CMT2D}. FIG. 4C shows the results of a GST pull-down to confirm that b1 domain of Nrp1 is the main binding site of GlyRS^{CMT2D}. The amount of GST and GST fusion proteins used for GlyRS^{CMT2D} binding was visualized by Ponceau staining. FIGS. 4D-4E show the results of an in vitro pull-down assay showing the mutual competition between L129P-GlyRS^{CMT2D} and VEGF-A165 for Nrp1 binding.

FIGS. 5A-5G show the results of detection of GlyRS proteins in the cell medium. FIG. 5A shows the results of Western-blot analysis of the GlyRS protein levels in NSC34 motor neurons. FIG. 5B shows the quantification of GlyRS protein level indicated in FIG. 5A. FIG. 5C shows the results of Western-blot analysis of C2C12 cell-differentiated myotubes. FIG. 5D shows the quantification of GlyRS protein level indicated in FIG. 5C. FIG. 5E shows the results of Western-blot analysis of undifferentiated C2C12 myoblasts. The level of GlyRS proteins in cell medium is diminished by application of the exosome-pathway inhibitor GW4869, but not by Brefeldin A (BFA), an inhibitor of the classical endoplasmic reticulum (ER) to Golgi secretory pathway. GAPDH (cytoplasmic protein), vWF (secretory protein through ER-Golgi pathway) and TSG101 (Exosomal protein) are used as controls. Data are presented as the mean±SEM of three independent experiments (*p<0.05, t-test). FIG. 5F shows the results of Western-blot analysis of the GlyRS protein level in NSC34 motor neurons. The level of GlyRS proteins in the cell medium is increased by the treatment of monensin (MON), an activator for microvesicle release by regulating the intracellular calcium level. Vehicle treated cells were used as control (Ctrl). FIG. 5G shows the results of Western-blot analysis of the GlyRS protein level in Cos7 cells transfected with plasmids encoding GlyRS^{WT} and P234KY-GlyRS^{CMT2D}. The expression of GlyRS proteins was detected by immuno-blot with antibody to V5 epitope tag. GAPDH was used as control. Note the similar level of GlyRS^{CMT2D} and GlyRS^{WT} in the media of transfected Cos7 cells. The observation that differentiated myotubes also secret GlyRS raises the possibility that muscles, which are directly innervated by the peripheral motor neurons, might contribute to the disease pathology.

FIGS. 6A-6B shows the detection of GlyRS proteins in exosome-enriched fractions. FIG. 6A is a schematic diagram showing a non-limiting procedure of “exosome” separation from the cell medium of NSC34 cells by differential centrifugation. FIG. 6B shows the results of Western-blot analysis of proteins associated with various fractions. GlyRS proteins were detected in the “exosome”-enriched fractions but not in supernatant fractions. The quality of the “exosome” preparation was controlled by detection of TSG101 (exosomal protein), Bip (ER-associated protein), GAPDH (cytoplasmic protein), and vWF (secretory protein through ER-Golgi pathway).

FIGS. 7A-7C depict that CMT2D mutant embryos have overall normal morphology but exhibit facial motor neuron migration defects. FIG. 7A shows a lateral view of WT and CMT2D mutant embryos at E12.5. Motor neurons are specifically labeled by a transgenic fluorescence reporter, Hb9:GFP. Note overall normal morphology of CMT2D mutant embryos (CMT) compared to their littermate controls (WT). FIG. 7B shows the results of Western-bolt analysis of protein expression in E12.5 mouse neural tissues. The expression levels of various neuronal proteins appear normal in CMT2D mutants compared to their littermate controls. FIG. 7C shows the quantification of the facial motor neuron migration phenotype, obtained by measuring the relative distance of the facial motor nucleus between WT and CMT littermate embryos (each dot represents one facial motor nucleus, n=6 embryos for WT; n=8 embryos for CMT2D). The migration of facial motor neurons is significantly disrupted in CMT embryos. Data are presented as the mean±SEM. **p<0.01 (t-test).

FIGS. 8A-8H show that Nrp1 is a genetic modifier of CMT2D. FIG. 8A shows hind limb extension test at 4 weeks. FIG. 8B graphically depicts the data gathered from FIG. 8A, showing the hind limb extension test at 4 weeks. ***p<0.001 (Mann-Whitney test). FIG. 8C shows the hind limb footprints of WT and mutant animals at 4 weeks. GarsCMT2D/Nrp1+/− mutant mice exhibit disrupted gait patterns of different degrees (mild, severe). Note that severe cases show inability to walk. FIG. 8D graphically depicts the stride length of WT and mutant animals at 4 weeks. FIG. 8E shows the neuromuscular junction (NMJ) immunostaining in the gastrocnemius muscles of 4-week-old mice with the motor nerve terminal and acetylcholine receptors on the muscle labeled highlighted in light grey. Scale bar represents 50 μm. FIG. 8F graphically shows the results of neuromuscular junction (NMJ) immunostaining in the gastrocnemius muscles of 4-week-old mice. Data are presented as mean values±SEM. n=3 mice per group. FIG. 8G shows myelinated axons from sciatic nerves of 4-week-old mice. FIG. 8H provides a histogram showing the quantification of axon numbers with the diameter larger than 2 μm. n=3 mice per group. *p<0.05, **p<0.01 (t-test).

FIGS. 9A-9C shows the genetic-interaction between Gars and Nrp1 in the early stage of CMT2D. FIG. 9A shows a hind limb extension test of wild-type and mutant animals at 2 weeks. FIG. 9B provides graphical data showing the Hind limb extension test of wild-type and mutant animals at 2 weeks. Note that 2 out of 9 Gars^{CMT2D}:Nrp1^{+/+} (CMT;Nrp1^{+/-}) mutants exhibit hind limb weakness with significantly lower scores compared to Gars^{CMT2D} (CMT), Nrp1^{+/-} (Nrp1^{+/-}) and wild-type (WT) littermate controls. FIG. 9C provides a comparison of stride lengths in different CMT2D mutant mice at 4-week-old: Gars^{CMT2D} (CMT), Gars^{CMT2D};TrkB^{+/-} (CMT;TrkB^{+/-}), Gars^{CMT2D};DCC^{+/-} (CMT;DCC^{+/-}), Gars^{CMT2D};Robo1^{+/-} (CMT;Robo1^{+/-}), and Gars^{CMT2D}; Unc5C^{+/-} (CMT;Unc5C^{+/-}). No significant differences were observed between compound heterozygotes and their littermate controls (CMT).

FIGS. 10A-10D shows the axonal dystrophy in CMT2D mice. FIG. 10A provides a histogram showing the axonal diameter frequencies in the sciatic nerves of 4-week-old wild-type (WT). FIG. 10B provides a histogram showing the axonal diameter frequencies in the sciatic nerves of 4-week-old Nrp1 heterozygous (Nrp1^{+/-}). FIG. 10C provides a histogram showing the axonal diameter frequencies in the sciatic nerves of 4-week-old Gars^{CMT2D} (CMT). FIG. 10D provides a histogram showing the axonal diameter frequencies in the sciatic nerves of 4-week-old (CMT;Nrp1^{+/-}) mutant mice. n=3 mice per group. Note the decreased numbers of larger-diameter axons in CMT;Nrp1^{+/-} mutants compared to CMT, Nrp1 heterozygous, and wild-type controls.

FIGS. 11A-11D show that VEGF treatment improves motor function in CMT2D mice. FIG. 11A illustrates a bilateral intramuscular injection of lentivirus (LV) into mouse hind limbs at P5. FIG. 11B provides the results of an inclined plane test of 4-week-old animals. ***p<0.001 (t-test). FIG. 11C provides the results of walking strides of 7-week-old animals. *p<0.05 (t-test). FIG. 11D provides the results of rotarod test of 2-month-old animals. **p<0.01 (t-test).

FIG. 12 shows the expression level of VEGF in mouse muscles. The expression level of VEGF proteins in muscle fibers of mice injected with lentivirus expressing LV-VEGF165-ires-GFP versus LV-GFP was determined by immunostaining with anti-VEGF antibodies. Note the expression level of VEGF in LV-VEGF infected muscles is significantly higher than in LVGFP infected control groups.

FIGS. 13A-13G shows that VEGF treatment retains limb strength in CMT2D mice. FIG. 13A shows that lentiviral vectors encoding GFP (LV-GFP) or VEGF-A165 (LVVEGF165) are injected unilaterally into each hind limb of the same GlyRS^{CMT2D} mutant mouse at P5. FIG. 13B shows that no significant difference was observed between both injected legs of wild type animals in the hind limb extension test. FIG. 13C shows that at 5 weeks, LV-GFP-injected legs (L, left) of CMT2D animals have largely lost their ability to extend, while LV-VEGF165-treated legs (R, right) retained more limb strength with significantly higher scores in the hind limb extension test (3 out of 7 animals). p<0.05 (Permutation test). FIG. 13D shows that there is no significant difference was observed between both injected legs of wild type animals in the hind limb extension test. FIG. 13E shows that at 5 weeks, LV-GFP-injected legs (L, left) of CMT2D animals have largely lost their ability to extend, while LV-VEGF165-treated legs (R, right) retained more limb strength with significantly higher scores in the hind limb extension test (3 out of 7 animals). p<0.05 (Permutation test). FIG. 13F shows that GDNF and VEGF-A121 treatments fail to improve stride length in CMT2D mice. Walking strides of 2-month-old CMT2D mice bilaterally injected with lentiviral vectors (LV) encoding GFP, GDNF or VEGF-A121. No significant difference of hind limb stride length was observed between animals treated with LV-GDNF, LVVEGF-A121, and LV-GFP controls. FIG. 13G shows that GDNF and VEGF-A121 treatments fail to improve stride length in CMT2D mice. Walking strides of 2-month-old CMT2D mice bilaterally injected with lentiviral vectors (LV) encoding GFP, GDNF or VEGF-A121. No significant difference of hind limb stride length was observed between animals treated with LV-GDNF, LVVEGF-A121, and LV-GFP controls.

FIG. 14 shows a schematic illustration of a non-limiting exemplary model for the neomorphic binding activity of GlyRS^{CMT2D}. Left panel, GlyRS^{WT} is a multifunctional protein with both intracellular and extracellular distributions. VEGF/Nrp1 signaling is an essential pathway for survival and function of motor neurons. (Note that VEGF may also act synergistically with other trophic factors, and/or maintains motor-function indirectly by acting on Nrp1 receptors on non-motor neurons.) Right panel, CMT2D mutations alter the conformation of GlyRS, enabling GlyRS^{CMT2D} to bind Nrp1. This aberrant interaction antagonizes the binding of VEGF to Nrp1, contributing to motor defects in CMT2D. The results do not exclude the possibility that GlyRS^{CMT2D} may also interact with other extracellular and/or intracellular targets, related to CMT2D pathology.

FIG. 15 shows the lack of Nrp1 interaction for non-tRNA synthetase genes linked to CMT, detected by co-immunoprecipitation in NSC-34 cells. Two MFN2 mutations, causing CMT2A, and two HSPB1 mutations, causing CMT2F, were utilized to detect Nrp1 interaction. HDAC6 and Daxx, which have been reported to interact with MFN2 and HSPB1, respectively, were utilized as the positive control. The result suggests that aberrant Nrp1 interaction is unique to tRNA synthetase-linked CMT.

FIG. 16 shows the aberrant gain-of-interaction of CMT-causing mutant tRNA synthetases is not common to any tRNA synthetase interacting proteins. As detected by co-immunoprecipitation of elongation factor eEF1A1 in NSC-34 cells, CMT-causing mutants of GlyRS, TyrRS, AlaRS, and HisRS do not show gain-of-interaction with eEF1A1. eEF1A1 is known to interact with all tRNA synthetases to take the aminoacylated tRNA from the synthetases to the ribosome for protein synthesis.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

aaRSs are essential housekeeping proteins that catalyze the aminoacylation of tRNA molecules during protein translation. Mutations in the aaRS gene, including GlyRS, TyrRS, AlaRS, HisRS, LysRS and MetRS, are found to be associated with CMT, and oftentimes result in loss-of-function features, suggesting that tRNA-charging deficits play a role in disease pathogenesis. For example, despite the broad requirement of GlyRS for protein biosynthesis in all cells, mutations in GARS cause a selective degeneration of peripheral axons leading to deficits in distal motor function. CMT is presently incurable, and although it is a rare disease, it is one of the commonest inherited neurological disorders, affecting 1 in 2,500 people. Diagnosis is laborious, costly, and inefficient. Furthermore, current diagnosis is poor for early stage detection. In addition, few effective treatments are available, which potentially show greatest efficacy during the early stages of disease state.

As described herein, mutated aaRS plays an important role in the interaction between VEGF and Nrp1. VEGF is thought to protect neurons from a variety of damaging insults, and deficiency in VEGF signaling can result in selective degeneration of motor neurons. As described herein, the VEGF-Nrp1 interaction is important for normal Nrp1 signaling. As shown herein, mutated aaRS, such as mutated GlyRS, antagonizes the VEGF-Nrp1 interaction, disrupting the normal signaling, by competing with VEGF for the binding site on Nrp1. As described herein, VEGF treatment is capable of ameliorating the loss of motor function in CMT subjects by displacing the mutated aaRS (e.g., GlyRS) from the Nrp1 binding site.

### Definitions

Unless defined otherwise, technical and scientific terms used herein have the same meaning as commonly understood by one of ordinary skill in the art to which the present disclosure belongs. See, e.g. Singleton et al., Dictionary of Microbiology and Molecular Biology 2nd ed., J. Wiley & Sons (New York, N.Y. 1994); Sambrook et al., Molecular Cloning, A Laboratory Manual, Cold Springs Harbor Press (Cold Springs Harbor, N.Y. 1989). For purposes of the present disclosure, the following terms are defined below.

As used herein, the term “treatment” refers to an intervention made in response to a disease, disorder or physiological condition manifested by a subject, particularly a subject suffering from one or more CMT diseases and/or one or more CMT-related neurological diseases. The aim of treatment may include, but is not limited to, one or more of the alleviation or prevention of symptoms, slowing or stopping the progression or worsening of a disease, disorder, or condition and the remission of the disease, disorder or condition. In some embodiments, “treatment” refers to both therapeutic treatment and prophylactic or preventative measures. Those in need of treatment include those already affected by a disease or disorder or undesired physiological condition as well as those in which the disease or disorder or undesired physiological condition is to be prevented. For example, in some embodiments, treatments reduce, alleviate, or eradicate the symptom(s) of the disease(s). As used herein, the term “prevention” refers to any activity that reduces the burden of the individual later expressing disease symptoms. This can take place at primary, secondary and/or tertiary prevention levels, wherein: a) primary prevention avoids the development of symptoms/disorder/condition; b) secondary prevention activities are aimed at early stages of the condition/disorder/symptom treatment, thereby increasing opportunities for interventions to prevent progression of the condition/disorder/symptom and emergence of symptoms; and c) tertiary prevention reduces the negative impact of an already established condition/disorder/symptom by, for example, restoring function and/or reducing any condition/disorder/symptom or related complications.

“Pharmaceutically acceptable” carriers are ones which are nontoxic to the cell or mammal being exposed thereto at the dosages and concentrations employed. “Pharmaceutically acceptable” carriers can be, but not limited to, organic or inorganic, solid or liquid excipients which is suitable for the selected mode of application such as oral application or injection, and administered in the form of a conventional pharmaceutical preparation, such as solid such as tablets, granules, powders, capsules, and liquid such as solution, emulsion, suspension and the like. Often the physiologically acceptable carrier is an aqueous pH buffered solution such as phosphate buffer or citrate buffer. The physiologically acceptable carrier may also comprise one or more of the following: antioxidants including ascorbic acid, low molecular weight (less than about 10 residues) polypeptides, proteins, such as serum albumin, gelatin, immunoglobulins; hydrophilic polymers such as polyvinylpyrrolidone, amino acids, carbohydrates including glucose, mannose, or dextrins, chelating agents such as EDTA, sugar alcohols such as mannitol or sorbitol, salt-forming counterions such as sodium, and nonionic surfactants such as Tween™, polyethylene glycol (PEG), and Pluronics™. Auxiliary, stabilizer, emulsifier, lubricant, binder, pH adjustor controller, isotonic agent and other conventional additives may also be added to the carriers.

The pharmaceutically acceptable or appropriate carrier may include other compounds known to be beneficial to an impaired situation of the GI tract, (e.g., antioxidants, such as Vitamin C, Vitamin E, Selenium or Zinc); or a food composition. The food composition can be, but is not limited to, milk, yogurt, curd, cheese, fermented milks, milk based fermented products, ice-creams, fermented cereal based products, milk based powders, infant formulae, tablets, liquid bacterial suspensions, dried oral supplement, or wet oral supplement.

As used herein, the term “antibody” includes polyclonal antibodies, monoclonal antibodies (including full length antibodies which have an immunoglobulin Fc region), antibody compositions with polyepitopic specificity, multispecific antibodies (e.g., bispecific antibodies, diabodies, and single-chain molecules, and antibody fragments (e.g., Fab or F(ab′)₂, and Fv). For the structure and properties of the different classes of antibodies, see e.g., Basic and Clinical Immunology, 8th Edition, Daniel P. Sties, Abba I. Terr and Tristram G. Parsolw (eds), Appleton & Lange, Norwalk, Conn., 1994, page 71 and Chapter 6.

The articles “a” and “an” are used herein to refer to one or to more than one (i.e., to at least one) of the grammatical object of the article. By way of example, “an element” means one element or more than one element.

By “about” is meant a quantity, level, value, number, frequency, percentage, dimension, size, amount, weight or length that varies by as much as 30, 25, 20, 15, 10, 9, 8, 7, 6, 5, 4, 3, 2 or 1% to a reference quantity, level, value, number, frequency, percentage, dimension, size, amount, weight or length.

The term “antagonist” includes to a molecule that reduces or attenuates a CMT-associated non-canonical biological activity a GlyRS polypeptide, such as a GlyRS mutant associated with CMT. Antagonists may include proteins such as antibodies, nucleic acids, carbohydrates, small molecules, or any other compound or composition that modulates the activity of a GlyRS mutant or its binding partner, either by directly interacting with the GlyRS mutant or its binding partner or by acting on components of the biological pathway in which the GlyRS mutant participates. Included are partial and full antagonists.

Throughout this specification, unless the context requires otherwise, the words “comprise,” “comprises,” and “comprising” will be understood to imply the inclusion of a stated step or element or group of steps or elements but not the exclusion of any other step or element or group of steps or elements.

By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

In some embodiments, the “purity” of any given agent (e.g., antibody, polypeptide binding agent) in a composition may be specifically defined. For instance, certain compositions may comprise an agent that is at least 80, 85, 90, 91, 92, 93, 94, 95, 96, 97, 98, 99, or 100% pure, including all decimals in between, as measured, for example and by no means limiting, by high pressure liquid chromatography (HPLC), a well-known form of column chromatography used frequently in biochemistry and analytical chemistry to separate, identify, and quantify compounds.

As used herein, the terms “function” and “functional” and the like refer to a biological, enzymatic, or therapeutic function.

The term “isolated” is meant material that is substantially or essentially free from components that normally accompany it in its native state. For example, an “isolated polynucleotide,” as used herein, includes a polynucleotide that has been purified from the sequences that flank it in its naturally-occurring state, e.g., a DNA fragment which has been removed from the sequences that are normally adjacent to the fragment. Alternatively, an “isolated peptide” or an “isolated polypeptide” and the like, as used herein, includes the in vitro isolation and/or purification of a peptide or polypeptide molecule from its natural cellular environment, and from association with other components of the cell; i.e., it is not significantly associated with in vivo substances.

The term “neomorphic region” as used herein relates to an exposed region or surface of human aaRS associated with one or more dominant, non-canonical activities of a neuronal disease-associated aaRS mutant. These neomorphic regions or surfaces are mostly or entirely hidden (e.g., they have reduced solvent exposure) in a properly folded wild-type aaRS sequence, but show significantly increased solvent exposure due to altered folding of a neuronal disease-associated aaRS mutant, such as a CMT-associated aaRS mutant. Certain neomorphic “opened up” regions partially overlap with the dimerization interface, and provide a new surface for potential pathological interactions specific to neuronal diseases such as CMT. Examples of disease-associated GlyRS mutants are described elsewhere herein and known in the art. Non-limiting examples of neomorphic regions include A57-A663, A57-A83, L129-D161, N208-Y320, V366-H378, P518-531, L584-Y604, F620-R635, and D654-A663 of human GlyRS, and fragments of said region(s), including antigenic fragments. Antigenic fragments of a neomorphic region can be at least about 6-12 to 20 or more residues in length, including all integers in between. Also included are combinations of these neomorphic regions, such as A57-D161, A57-Y320, A57-H378, A57-M531, A57-Y604, A57-R635, L129-Y320, L129-H378, L129-M531, L129-Y604, L129-R635, L129-A663, N208-H378, N208-53I, N208-Y604, N208-R635, N208-A663, V366-M531, V366-Y604, V366-R635, V366-A663, P518-Y604, P518-R635, P518-A663, L584-R635, L584-A663, and F620-A663, and others, including fragments thereof. As used herein, the numbering of the residues used herein is based on the cytoplasmic GlyRS sequence, and the numbering needs to be increased by 54 when a mitochondrial GlyRS protein is considered.

Examples of specific fragments of neomorphic regions include F79-A83, F78-T137, I108-E123, F224-L242, M227-L257, I232-N253, L252-E291, L258-R288, F147-K150, E515-M531, and R635-I645. Examples of specific neomorphic regions associated with GlyRS mutants include A57-A83, G97-T110, E119-S178, N208-Y320, A326-N348, L361-H378, K423-E429, V461-Y464, L480-F486, K505-P554, V564-N570, L584-Y604, F620-1645, and D654-A663 for the L129P of GlyRS; A57-A83, G97-E123, F147-L189, F204-Y320, N348-H378, V461-Y464, K483-M531, D545-R642, and D654-E685 for the G240R mutant of GlyRS; A57-A83, L129-K150, S183-V188, N208-Y320, N348-D389, K423-E429, L480-E485, D500-L511, P518-M531, T538-F550, L584-Y604, F620-1645, D654-A663 for the G526R mutant of GlyRS; A57-107, L129-D161, N208-Y320, V366-1402, K493-Q496, V513-M531, A555-R635, and D654-E685 for the S581L mutant of GlyRS; and A57-N106, L129-L203, N208-Y320, V366-D389, A421-Y464, E504-M531, F551-I645, and D654-A663 for the G598A mutant of GlyRS. For instance, regions characterized as “31-50%” or “>51%” can be included as neomorphic regions. Regions characterized as “29-6%” can also be characterized as neomorphic regions.

“Non-canonical” activity as used herein, refers generally to an activity possessed by a GlyRS polypeptide that is other than aminoacylation and, more specifically, other than the addition of its cognate amino acid onto its cognate tRNA molecule. Non-limiting examples of non-canonical activities include extracellular signaling, RNA-binding, modulation of cell proliferation, modulation of cell migration, modulation of cell differentiation, modulation of apoptosis or other forms of cell death, modulation of cell signaling, modulation of cell binding, modulation of cellular metabolism, modulation of cytokine production or activity, modulation of cytokine receptor activity, modulation of inflammation, and the like. Certain of these non-canonical activities may be related to the pathology of various diseases described herein and known in the art, such as CMT and Distal Spinal Muscular Atrophy Type V (dSMA-V), including, for example, activities related to neurite distribution defects and axonal degeneration, among others. Some non-canonical activities of disease-associated GlyRS relate to modulating (e.g., reducing, enhancing) the activity or activation of neuropilin transmembrane receptors, such as Nrp1, and/or modulating the activity of one or more of neuropilin ligands by altering (e.g., inhibiting) their interaction with neuropilin. Examples of such ligands include vascular endothelial growth factors (VEGFs), hepatocyte growth factor, placental growth factors (PGFs), semaphorins, among others described herein and known in the art. Specific neuropilin ligands include the VEGF-165 isoform, VEGF-A, VEGF-B, the PLGF-2 isoform, and semaphorin-3A. One specific non-canonical activity of disease-associated GlyRS includes the inhibition of neuropilin-induced neurite outgrowth in cells.

The term “half maximal effective concentration” or “EC₅₀” refers to the concentration of an antibody or other agent described herein at which it induces a response halfway between the baseline and maximum after some specified exposure time, the EC₅₀ of a graded dose response curve therefore represents the concentration of a compound at which 50% of its maximal effect is observed. In some embodiments, the EC₅₀ of an agent provided herein is indicated in relation to a “non-canonical” activity, as noted above, for example, a non-canonical activity related to symptoms or pathology of CMT. EC₅₀ also represents the plasma concentration required for obtaining 50% of a maximum effect in vivo. Similarly, the “EC₉₀” refers to the concentration of an agent or composition at which 90% of its maximal effect is observed. The “EC₉₀” can be calculated from the “EC₅₀” and the Hill slope, or it can be determined from the data directly, using routine knowledge in the art. In some embodiments, the EC₅₀ of an antibody or other agent is less than about 0.01, 0.05, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 40, 50, 60, 70, 80, 90, or 100 nM. Preferably, biotherapeutic compositions will have an EC₅₀ value of about 1 nM or less.

The term “modulating” includes “increasing” or “stimulating,” as well as “decreasing” or “reducing,” typically in a statistically significant or a physiologically significant amount as compared to a control. An “increased” or “enhanced” amount is typically a “statistically significant” amount, and may include an increase that is 1, 2, 3, 4, 5, 6, 7, 8, 9, 10, 15, 20, 30 or more times (e.g., 500, 1000 times) (including all integers and decimal points in between and above 1, e.g., 1.5, 1.6, 1.7, 1.8, etc.) the amount produced by no composition (the absence of an agent or compound) or a control composition. A “decreased” or reduced amount is typically a “statistically significant” amount, and may include a 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, or 100% decrease in the amount produced by no composition (the absence of an agent or compound) or a control composition, including all integers in between. As one non-limiting example, a control in comparing canonical and non-canonical activities could include the activity (e.g., antagonist activity) or binding specificity of an antibody or binding agent towards a disease-associated GlyRS mutant of interest relative to a wild-type human GlyRS. Other examples of “statistically significant” amounts are described herein.

The terms “sequence identity” or, for example, comprising a “sequence 50% identical to,” as used herein, refer to the extent that sequences are identical on a nucleotide-by-nucleotide basis or an amino acid-by-amino acid basis over a window of comparison. Thus, a “percentage of sequence identity” may be calculated, for example, by comparing two optimally aligned sequences over the window of comparison, determining the number of positions at which the identical nucleic acid base (e.g., A, T, C, G, U) or the identical amino acid residue (e.g., Ala, Pro, Ser, Thr, Gly, Val, Leu, Ile, Phe, Tyr, Tip, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity.

The term “solubility” refers to the property of an antibody, peptide, or other agent provided herein to dissolve in a liquid solvent and form a homogeneous solution. Solubility is typically expressed as a concentration, either by mass of solute per unit volume of solvent (g of solute per kg of solvent, g per dL (100 mL), mg/ml, etc.), molarity, molality, mole fraction or other similar descriptions of concentration. The maximum equilibrium amount of solute that can dissolve per amount of solvent is the solubility of that solute in that solvent under the specified conditions, including temperature, pressure, pH, and the nature of the solvent. In some embodiments, solubility is measured at physiological pH. In some embodiments, solubility is measured in water or a physiological buffer such as PBS. In some embodiments, solubility is measured in a biological fluid (solvent) such as blood or serum. In some embodiments, the temperature can be about room temperature (e.g., about 20, 21, 22, 23, 24, 25° C.) or about body temperature (37° C.). In some embodiments, an agent has a solubility of at least about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, or 30 mg ml at room temperature or at 37° C.

As used herein, the term “subject” can be an animal, such as a vertebrate, preferably a mammal. The term “mammal” is defined as an individual belonging to the class Mammalia and includes, without limitation, humans, domestic and farm animals, and zoo, sports, or pet animals, such as sheep, dogs, horses, cats or cows. In some embodiments, the subject is mouse or rat. In some embodiments, the subject is human. A “subject” includes any animal that exhibits a symptom, or is at risk for exhibiting a symptom, of one or more diseases such as distal spinal muscular atrophies (dSMA) and distal hereditary motor neuropathies (dHMN), which are preferably associated with one or more aaRS mutations. Examples of neuronal disease-associated GlyRS mutants include, without limitation, A57V, E71G, L129P, D146N, P234KY, G240R, P244L, E279D, I280F, H418R, D500N, G526R, S581L, and G598A mutants of wild-type GlyRS. Specific examples of diseases, including mutant GlyRS-associated diseases, include CMT Type 1, CMT Type 2, CMT Type 2D, and dSMA Type V, among others described herein and known in the art. Also included are subjects for which it is desirable to profile presence and/or levels of disease-associated GlyRS mutants, for diagnostic or other purposes. In certain aspects, a subject includes any animal having a disease or condition associated with increased or aberrant activity of a neuropilin-related pathway, as described herein and known in the art. Suitable subjects (patients) include laboratory animals (such as mouse, rat, rabbit, or guinea pig), farm animals, and domestic animals or pets (such as a cat or dog). Non-human primates and, preferably, human patients, are included.

The term “detecting” is used herein in the broadest sense to include both qualitative and quantitative measurements of a target molecule. In one aspect, the detecting method as described herein is used to identify the mere presence of VEGF in a biological sample. In another aspect, the method is used to test whether VEGF in a sample is at a detectable level. In yet another aspect, the method can be used to quantify the amount of VEGF in a sample and further to compare the VEGF levels from different samples.

The term “biological sample” used herein refers to a sample from an animal, but preferably is from a mammal, more preferably from a human. For example, the biological sample can be a sample obtained directly from an animal, including but not limited to, a biofluidic sample, a neural tissue, a blood sample, or any combination thereof. The biological sample can also be a sample derived from materials obtained directly from an animal. For example, the biological sample may be, or comprise, progenies of cells (e.g., neuronal cells) obtained from the animal. The progenies can be obtained by conventional methods known in the art, for example cell culturing. Biological samples suitable for methods described herein can be or can comprise, but not limited to, neural tissue, neural cells, neuroglia cells, peripheral blood, lymphoblastoid cells, cerebrospinal fluid, ependymal cells, muscle tissue, muscle cells, skin tissues, fibroblasts, or any combination thereof. In some embodiments, the biological sample comprises one or more neuronal cells. In some embodiments, the biological sample is from a patient with a CMT disease and/or a CMT-related neurological disease. Such samples include samples where the presence of Nrp1 is sufficient for detection.

The term “capture reagent” refers to a reagent capable of binding and/or capturing a target molecule in a sample such that under suitable condition, the capture reagent-target molecule complex can be separated from the rest of the sample. In some embodiments, the capture reagent is immobilized or immobilizable on a solid support. In some embodiments, a sandwich immunoassay is described, wherein the capture reagent is preferably Nrp1 or a fragment thereof for use in binding VEGF or mutated GlyRS.

The term “detectable antibody” refers to an antibody that is capable of being detected either directly through a label amplified by a detection means, or indirectly through, e.g., another antibody that is labeled. For direct labeling, the antibody is typically conjugated to a moiety that is detectable by some means. The preferred detectable antibody is biotinylated antibody.

The term “detection means” refers to a moiety or technique used to detect the presence of the detectable antibody in the ELISA herein and includes detection agents that amplify the immobilized label such as label captured onto a microtiter plate. Preferably, the detection means is a fluorometric detection agent such as avidin or streptavidin.

The practice of the present disclosure will employ, unless indicated specifically to the contrary, conventional methods of molecular biology and recombinant DNA techniques within the skill of the art, many of which are described below for the purpose of illustration. Such techniques are explained fully in the literature. See, e.g., Sambrook, el al, Molecular Cloning: A Laboratory Manual (3^{rd} Edition, 2000); DNA Cloning: A Practical Approach, vol. 1 & II (D. Glover, ed.); Oligonucleotide Synthesis (N. Gait, ed., 1984); Oligonucleotide Synthesis: Methods and Applications (P. Herdewijn, ed., 2004); Nucleic Acid Hybridization (B. Hames & S. Higgins, eds., 1985); Nucleic Acid Hybridization: Modern Applications (Buzdin and Lukyanov, eds., 2009); Transcription and Translation (B. Hames & S. Higgins, eds., 1984); Animal Cell Culture (R. Freshney, ed., 1986); Freshney, R. I. (2005) Culture of Animal Cells, a Manual of Basic Technique, 5^{th} Ed. Hoboken N.J., John Wiley & Sons; B. Perbal, A Practical Guide to Molecular Cloning (3^{rd} Edition 2010); Farrell, R., RNA Methodologies: A Laboratory Guide for Isolation and Characterization (3^{rd} Edition 2005).

### Hereditary Peripheral Neuropathy

The terms “neuropathy” or “neuropathies,” as used herein, is defined as a functional defect or defects and/or a pathological change or changes in the peripheral nervous system. In some embodiments, the peripheral neuropathy is an autosomal recessive form of the Charcot-Marie-Tooth (CMT) syndrome or CMT-related neurological diseases.

Charcot-Marie-Tooth (CMT) disease, also known as hereditary motor and sensory neuropathy, was named after three physicians who first described the disease in 1886. The disease is characterized by loss of muscle tissue and touch sensation in body extremities, predominantly in the feet and legs but also in the hands and arms. Presently incurable, this disease is one of the most common inherited neurological disorders affecting one in 2,500 people. Genetically, CMT disease is a heterogeneous group of disorders, which has more than 90 subtypes, each subtype linked to mutations in a specific gene. Different subtypes of CMT have similar clinical presentations, but likely different disease-causing mechanisms. Six aminoacyl-tRNA synthetases (aaRS) genes, including glycyl-tRNA synthetase (GlyRS or GARS), tyrosyl-tRNA synthetase (TyrRS or YARS), histidyl-tRNA synthetase (HisRS or HARS), alanyl-tRNA synthetase (AlaRS or AARS), lysyl-tRNA synthetase (LysRS or KARS), and methionyl-tRNA synthetase (MetRS or MARS) have been linked to CMT. However, so far only the first four genes/subtypes (GlyRS/CMT2D, TyrRS/DI-CMTC, AlaRS/CMT2N, and HisRS/CMT2W) have strong genetic evidence as CMT-associative. In most cases, the disease-causing mutation is dominant, and thus implicates a neomorphic (gain of gene function that is different from the normal function) form(s) that engenders the neuropathology.

In view of the high heterogeneity of CMT diseases, diagnosis of the correct subtype of CMT can be critical for developing effective treatments for CMT patients. Current diagnoses of aaRS linked CMT only rely on genetic association studies, which cannot be performed effectively without a large family of patients. As disclosed herein, aberrant Nrp1-aaRS (e.g., GlyRS, TyrRS, AlaRS, and HisRS) interaction is present in patients suffering from various CMT subtype diseases. The detection of the aberrant interaction can be used to diagnose CMT diseases, including CMT diseases in the absence of strong genetic association. The methods, compositions and kits disclosed herein allow effective, sensitive and accurate diagnoses of CMT diseases and CMT-related neurological diseases.

Aminoacyl-tRNA Synthetase (aaRS)

aaRSs are a family of essential enzymes in translation (Ling et al., Annu. Rev. Microbiol. 63:61-78 (2009)). Each member is responsible for charging one specific amino acid onto its cognate tRNAs. The charged tRNAs then use the embedded 3-nucleotide anticodons to decode mRNA and provide the corresponding amino acid building blocks for protein synthesis on the ribosome. Six aaRS genes, including glycyl-tRNA synthetase (GlyRS or GARS), tyrosyl-tRNA synthetase (TyrRS or YARS), histidyl-tRNA synthetase (HisRS or HARS), alanyl-tRNA synthetase (AlaRS or AARS), lysyl-tRNA synthetase (LysRS or KARS), and methionyl-tRNA synthetase (MetRS or MARS) have been linked to CMT. Four genes/subtypes, GlyRS/CMT2D, TyrRS/DI-CMTC, AlaRS/CMT2N, and HisRS/CMT2W, have strong genetic evidence as CMT-associative.

Glycyl-tRNA synthetase (GlyRS) was the first tRNA synthetase implicated in CMT (Antonellis et al., Am. J. Hum. Genet. 72:1293-1299 (2003)). Eleven different missense mutations of GARS have been reported to cause a dominant axonal form of CMT (CMT type 2D) in patients (He et al., Proc. Natl. Acad. Sci. 108: 12307-12312 (2011)). Two separate spontaneous or ENU-induced missense mutations have also been linked to CMT-like phenotypes in mice. Interestingly, not all mutations affect the aminoacylation activity of the tRNA synthetase. Furthermore, studies in mice clearly demonstrate that the CMT-like phenotype was not caused by haploinsufficiency in protein synthesis, but rather by a pathogenic role of the mutant GlyRS itself, which remains to be defined at the molecular level. GlyRS is a class II tRNA synthetase, whose catalytic domain consists of a central antiparallel β sheet flanked with a helices, and three conserved sequence motifs (motifs 1-3). Human GlyRS has three insertions that split the catalytic domain, a metazoan-specific helix-turn-helix WHEP domain, and an anticodon binding domain at the N- and C-terminal side of the catalytic domain, respectively. Like most class II tRNA synthetases, GlyRS functions as a dimer for aminoacylation. Interestingly, despite being well-separated in the primary sequence of the three domains of GlyRS, all known CMT-causing mutations are located near the dimer interface of crystal structure (He et al., Proc. Natl. Acad. Sci. 108: 12307-12312 (2011)). This observation suggests a connection of the dimer interface with the disease-causing mechanism. However, different CMT-causing mutations have different effects on dimer formation: some disrupt, some strengthen and some seem to have no effect on the dimer. In addition, crystal structures of two CMT-causing mutant proteins showed little difference from that of the WT protein, and suggest that structural differences, if any, between mutant and WT GlyRSs are subtle and could be suppressed by crystal packing forces.

Mutations in GlyRS (GlyRS^{CMT2D}) alter the conformation of GlyRS, enabling GlyRS^{CMT2D} to bind Nrp1. In some embodiments, GlyRS^{CMT2D} is a missense GlyRS mutant. In some embodiments, the missense GlyRS^{CMT2D} mutant comprises at least one amino acid substitution selected from the group consisting of E71G, P234KY, L129P, S211F, G240R, E279D, H418R, G526R, and a combination thereof.

The GlyRS mutant, in some embodiments, comprises at least one amino acid substitution selected from the group consisting of A57V, E71G, P234KY, L129P, D146N, C157R, S211F, L218Q, G240R, P244L, E279D, I280F, H418R, D500N, G526R, S581L, G598A, and a combination thereof. The numbering of the residues is based on the cytoplasmic GlyRS sequence and requires the addition of 54 amino acids when the mitochondrial GlyRS protein is considered.

In addition to GlyRS, mutations in other aaRS also play a significant role in CMT diseases, as described herein. For example, also contemplated herein are TyrRS mutants, for example TyrRS mutants comprising a 4 amino acid deletion of VKQV at positions 153-156, at least one amino acid substitution selected from the group consisting of G41R, Del153-156VKGV, D81I, E196K, or a combination thereof; AlaRS mutants, for example AlaRS mutants comprising at least one amino acid substitution selected from the group consisting of N71Y, G102R, R329H, E688G, E778A, D893N, and a combination thereof; HisRS mutants, for example HisRS mutants comprising at least one amino acid substitution selected from the group consisting of T132I, P134H, R137Q, D175E, D364Y, and a combination thereof; LysRS mutants, for example LysRS mutants comprising at least one amino acid substitution selected from the group consisting of L133H, Y173SerfsX7, I302M, T623S, and a combination thereof; and MetRS mutants, for example MetRS mutants comprising R618C, P800T, or a combination thereof. As used herein, the terms “a mutated aaRS”, “a mutant aaRS” and “an aaRS mutant” are used interchangeably, and refer to an aaRS protein having one or more amino acid addition, substitution, deletion, or a combination thereof relative to the corresponding wildtype aaRS protein. It has been found that mutated amino acids in various aaRS identified as CMT-associated are highly conserved across species. For example, Antonellis (2003) Am. J. Hum. Genet. 72:1293-1299 shows that E71, L129, G240, and G526 CMT-associated substitution in GlyRS are highly conserved; Jordanova et al. (2006) Nature Genetics 38(2):197-202 shows that G41, 153-156VKGV and E196 CMT-associated mutations in TyrRS are highly conserved; and McLaughlin et al. (2011) Human Mutation 33910:244-253 shows that N71, R329 and E778 CMT-associated mutations in AlaRS are highly conserved.

In some embodiments, the aaRS is glycyl-tRNA synthetase (GlyRS), including but not limited to the GlyRS protein having the amino acid sequence of SEQ ID NOs: 10-25; tyrosyl-tRNA synthetase (TyrRS), including but not limited to the TyrRS protein having the amino acid sequence of SEQ ID NOs: 26-30; alanyl-tRNA synthetase (AlaRS), including but not limited to the AlaRS protein having the amino acid sequence of SEQ ID NO: 31-37, histidyl-tRNA synthetase (HisRS), including but not limited to the HisRS having the sequence of SEQ ID NO: 38-43; lysyl-tRNA synthetase (LysRS), including but not limited to the LysRS protein having the amino acid sequence of SEQ ID NO: 44-48; or methionyl-tRNA synthetase (MetRS), including but not limited to the MetRS protein having the amino acid sequence of SEQ ID NO: 49-51. Non-limiting exemplary coding sequences for GlyRS, TyrRs, AlaRS, HisRS, LysRS, and MetRS are provided in SEQ ID NOs: 1-6, respectively.

### Vascular Endothelial Growth Factor (VEGF)

Vascular endothelial growth factors (VEGFs) regulate blood and lymphatic vessel development. They are predominantly produced by endothelial, hematopoietic and stromal cells in response to hypoxia and stimulation with growth factors such as transforming growth factors, interleukins and platelet-derived growth factor. VEGF is a heparin binding growth factor with a molecular weight of 45 kD (Plouet et al., EMBO J. 8:3801 (1989); Neufeld et al., Prog. Growth Factor Res. 5:89 (1994)). VEGF is produced by tissues and does not have to enter the circulation to exert its biological effect, but rather acts locally as a paracrine regulator. the ability to accurately measure VEGF will be important to understand its potential role(s). The ability to measure endogenous VEGF levels depends on the availability of sensitive and specific assays. Colorimetric, chemiluminescence, and fluorometric based enzyme-linked immunosorbent assays (ELISAs) for VEGF have been reported.

The term “VEGF” as used herein refers to the 165-amino acid vascular endothelial cell growth factor, and related 121-, 145-, 189-, and 206-amino acid vascular endothelial cell growth factors (Leung et al., Science 246:1306 (1989); Houck et al., Mol. Endocrin. 5:1806 (1991)), together with the naturally occurring allelic and processed forms of those growth factors. A non-limiting exemplary coding sequence and protein sequence of VEGF-A is provided herein as SEQ ID NO: 8, and SEQ ID NO: 54, respectively.

Various isoforms of VEGF have been shown to bind to neuropilin-1 (Soker et al., Cell 92:735-745 (1998)), including, for example, VEGF-A, and VEGF isoforms are capable of interacting of interacting with neuropilin. For example, VEGF binds to the b1 domain of Nrp1. As described herein, mutated aaRS, such as GlyRS^{CMT2D} also binds to the b1 domain of Nrp1, thereby competing for binding to Nrp1.

### Neuropilin 1 (Nrp1)

Neuropilins, including Neuropillin 1 (Nrp1), play a general role in axon guidance during the development of the nervous system in vertebrates, and play other important roles in normal physiology, this discovery suggests that disease-associated GlyRS mutants may mediate disease progression at least in part through the negative regulation of neuropilins. This discovery thus enables the development of specific screening assays to identify molecules such as antibodies or other binding agents that can block the interaction between disease-associated mutant aaRS (e.g., GlyRS) and neuropilins. It also suggests that soluble isoforms of neuropilins could sequester disease-associated aaRS (e.g., GlyRS) mutants, and thereby reduce the symptoms or progression of aaRS-associated diseases, such as CMT and other diseases mediated by aaRS mutants.

Additionally, because neuropilins play diverse roles during the physiological regulation of processes such as angiogenesis, axon guidance, cell survival, migration, and invasion, the ability of aaRS mutants to specifically interact with neuropilins further suggests that these aaRS mutants (or other molecules with exposed neomorphic regions of aaRS) may have therapeutic utility in their own right, for example, where physiological problems occur due to aberrant activity of neuropilins, aberrant activity of neuropilin ligands such as VEGF. A non-limiting exemplary coding sequence and protein sequence of Nrp1 is provided in SEQ ID NO: 7 and SEQ ID NO: 52, respectively. Moreover, a non-limiting exemplary coding sequence and protein sequence of Nrp1 b1 domain is shown in SEQ ID NO: 8 and SEQ ID NO: 53, respectively.

Methods of Detecting Mutant aaRS and/or VEGF in a Subject

Some embodiments herein relate to methods for detecting mutant aaRS, such as GlyRS^{CMT2D} and/or VEGF in a biological sample. The biological sample can be from a subject having or suspected of having a CMT disease and/or a CMT-related neurological disease. The detection of the mutant aaRS, including GlyRS^{CMT2D}, and/or VEGF can provide a diagnosis of CMT disease. Also provided herein in some embodiments are methods for determining the presence of a mutated aaRS in a biological sample. In some embodiments, the method comprises: providing a biological sample from a subject having or suspected of having a mutated aaRS; immobilizing a Nrp1 protein or a fragment thereof on a solid support; contacting the biological sample with the immobilized Nrp1 protein under conditions that allows binding of Nrp1 protein to an aaRS to form an immobilized Nrp1-aaRS complex on the solid support; contacting the solid support with a detectably labeled molecule that specifically binds the aaRS; and detecting the amount of labeled Nrp1-aaRS complex on the solid support as indicative of the presence or absence of the mutated aaRS in the subject.

Enzyme Linked Immune-Sorbent Assays (ELISA)

ELISA is a laboratory technique commonly used for measuring the concentration of an analyte (for example a protein) in a solution. As described herein, ELISA assays can be used to detect mutant aaRS, VEGF, Nrp1, or a combination thereof in the methods described herein. ELISA assays can be performed with variations. In some embodiments, cells in a biological sample are lysed to obtain free target molecule of interest (for example, VEGF or aaRS). For example, the biological sample may include endogenous Nrp1-containing complex having Nrp1 bound to either or both of VEGF and aaRS. In some embodiments, the lysis buffer is sufficiently strong to at least partially dissociate the target molecule from endogenous Nrp1, thereby providing free target molecule in the biological sample. In some embodiments, cells in the biological sample are not lysed. The biological sample can be any biological sample as described previously, from which a complex of Nrp1-aaRS and/or Nrp1-VEGF can be obtained. The biological sample can comprise, for example, neural tissue, neural cells, neuroglia cells, peripheral blood, lymphoblastoid cells, cerebrospinal fluid, ependymal cells, muscle tissue, muscle cells, skin tissues, fibroblasts, or any combination thereof.

In some embodiments, the biological sample is contacted and incubated with the immobilized capture (or coat) reagents, bound to a solid support. In some embodiments, the immobilized capture reagent is Nrp1 or fragments thereof, including a b1 domain of Nrp1. In some embodiments, the Nrp1 or fragment thereof comprises or consists of a b1 domain for specifically binding to the target molecule of interest (VEGF or aaRS). In some embodiments, the Nrp1 or fragment thereof comprises or consists of an amino acid sequence that is at least 60%, 70%, 80%, 90%, 95%, 98%, or 99% identical to the b1 domain of SEQ ID NO: 52 (i.e., SEQ ID NO: 53). In some embodiments, the Nrp1 or fragment thereof comprises or consists of a truncated Nrp1 b1 domain. For example, the Nrp1 or fragment thereof can comprise or consist of at least 50%, 60%, 70%, 80%, 90%, 95%, 98%, or 99% of the sequence of SEQ ID NO: 53. In some embodiments, the Nrp1 or fragment thereof comprises or consists of the amino acid sequence of SEQ ID NO: 53. In some embodiments, the Nrp1 or fragment thereof is a recombinant polypeptide. Immobilization can be accomplished by insolubilizing the capture reagents either before the assay procedure, as by adsorption to a water-insoluble matrix or surface or non-covalent or covalent coupling (for example, using glutaraldehyde or carbodiimide cross-linking, with or without prior activation of the support with, e.g., nitric acid and a reducing agent as is known, or afterward, e.g., by immunoprecipitation.

In some embodiments, the immobilized Nrp1 or fragment thereof binds to one or more mutated aaRS. In some embodiments, at least one of the one or more mutated aaRS is a mutated GlyRS, TyrRS, AlaRS, HisRS, LysRS, or MetRS. For example, the GlyRS mutant can comprise one or more of amino acid substitution A57V, E71G, P234KY, L129P, D146N, G157R, S211F, L218Q, G240R, P244L, E279D, I280F, H418R, D500N, G526R, S581L, and G598A, or a combination thereof. Non-limiting examples of mutant GlyRS protein sequences are provided in SEQ ID NOs: 11-25. The TyrRS mutant can, in some embodiments, comprise a 4 amino acid deletion of VKQV at positions 153-156, at least one amino acid substitution selected from the group consisting of G41R, D81I, E196K, or a combination thereof. Non-limiting examples of mutant TyrRS protein sequences are provided in SEQ ID NOs: 27-30. The AlaRS mutant can, in some embodiments, comprise at least one amino acid substitution selected from the group consisting of N71Y, G102R, R329H, E688G, E778A, D893N, and a combination thereof. Non-limiting examples of mutant GlyRS protein sequences are provided in SEQ ID NOs: 32-37. The HisRS mutant can, in some embodiments, comprise at least one amino acid substitution selected from the group consisting of T132I, P134H, R137Q, D175E, D364Y, and a combination thereof. Non-limiting examples of mutant HisRS protein sequences are provided in SEQ ID NOs: 39-43. The LysRS mutant can, in some embodiments, comprise at least one amino acid substitution selected from the group consisting of L133H, Y173SerfsX7, I302M, T623S, and a combination thereof. Non-limiting examples of mutant HisRS protein sequences are provided in SEQ ID NOs: 45-48. The MetRS mutant can, in some embodiments, comprise at least one amino acid substitution selected from the group consisting of R618C, P800T, and a combination thereof. Non-limiting examples of mutant MetRS protein sequences are provided in SEQ ID NOs: 50 and 51.

The solid phase used for immobilization may be any inert support or carrier that is essentially water insoluble and useful in immunometric assays, including supports in the form of, e.g., surfaces, particles, porous matrices, etc. Examples of commonly used supports include small sheets, Sephadex, polyvinyl chloride, plastic beads, and assay plates or test tubes manufactured from polyethylene, polypropylene, polystyrene, and the like including 96-well microtiter plates, as well as particulate materials such as filter paper, agarose, cross-linked dextran, and other polysaccharides. In some embodiments, the immobilized capture reagents are coated on a microtiter plate, a bead, or a combination thereof. In some embodiments, the solid phase used is a multi-well microtiter plate that can be used to analyze several samples at one time. In some embodiment, a microtest 96-well ELISA is used.

The solid phase is coated with the pre-mixed capture reagents as defined above, which may be linked by a non-covalent or covalent interaction or physical linkage as desired. If covalent, the plate or other solid phase is incubated with a cross-linking agent together with the capture reagent under conditions well known in the art such as for 1 hour at room temperature.

Commonly used cross-linking agents for attaching the pre-mixed capture reagents to the solid phase substrate include, e.g., 1,1-bis(diazoacetyl)-2-phenylethane, glutaraldehyde, N-hydroxysuccinimide esters, for example, esters with 4-azidosalicylic acid, homobifunctional imidoesters, including disuccinimidyl esters such as 3,3′-dithiobis (succinimidylpropionate), and bifunctional maleimides such as bis-N-maleimido-1,8-octane. Derivatizing agents such as methyl-3-[(p-azidophenyl)dithio]propioimidate yield photoactivatable intermediates capable of forming cross-links in the presence of light.

If 96-well plates are utilized, they can be coated with the mixture of capture reagents (typically diluted in a buffer such as 0.05 M sodium carbonate by incubation for at least about 10 hours, more preferably at least overnight, at temperatures of about 4-20° C., more preferably about 4-8° C., and at a pH of about 8-12, more preferably about 9-10, and most preferably about 9.6. If shorter coating times (1-2 hours) are desired, one can use 96-well plates with nitrocellulose filter bottoms (such as, for example, Millipore MULTISCREEN™) or coat at 37° C. The plates may be stacked and coated long in advance of the assay itself, and then the assay can be carried out simultaneously on several samples in a manual, semi-automatic, or automatic fashion, such as by using robotics.

The coated plates can then be treated with a blocking agent that binds non-specifically to and saturates the binding sites to prevent unwanted binding of the free ligand to the excess sites on the wells of the plate. Examples of appropriate blocking agents for this purpose include, e.g., gelatin, bovine serum albumin, egg albumin, casein, and non-fat milk. The blocking treatment typically takes place under conditions of ambient temperatures for about 1-4 hours, preferably about 1.5 to 3 hours.

After coating and blocking, the standard (purified VEGF or GlyRS^{CMT2D}) or the biological sample to be analyzed, appropriately lysed and/or diluted, is added to the immobilized phase. The preferred dilution rate is about 5-15%, preferably about 10%, by volume. Buffers that may be used for dilution for this purpose include (a) PBS containing 0.5% BSA, 0.05% TWEEN 20™ detergent (P20), 0.05% PROCLIN™ 300 antibiotic, 5 mM EDTA, 0.25% Chaps surfactant, 0.2% beta-gamma globulin, and 0.35M NaCl; (b) PBS containing 0.5% BSA, 0.05% P20, and 0.05% PROCLIN™ 300, pH 7; (c) PBS containing 0.5% BSA, 0.05% P20, 0.05% PROCLIN™ 300, 5 mM EDTA, and 0.35 M NaCl, pH 6.35; (d) PBS containing 0.5% BSA, 0.05% P20, 0.05% PROCLIN™ 300, 5 mM EDTA, 0.2% beta-gamma globulin, and 0.35 M NaCl; and (e) PBS containing 0.5% BSA, 0.05% P20, 0.05% PROCLIN™ 300, 5 mM EDTA, 0.25% Chaps, and 0.35 M NaCl. PROCLIN™ 300 acts as a preservative, and TWEEN 20™ acts as a detergent to eliminate non-specific binding.

For sufficient sensitivity, it can be beneficial that the amount of biological sample added be such that the immobilized capture reagents are in molar excess of the maximum molar concentration of VEGF or aaRS anticipated in the biological sample after appropriate dilution of the sample. This anticipated level depends mainly on any known correlation between the concentration levels of the VEGF or aaRS in the particular biological sample being analyzed with the clinical condition of the patient. Thus, for example, patients may have a maximum expected concentration of VEGF that is low compared to a healthy subject, because aaRS will displace the VEGF from binding to Nrp1 in the sample of CMT disease and/or CMT-related neurological disease subjects, resulting in lower than normal quantities of VEGF.

On the other hand, in an ELISA assay used for the detection of aaRS, such as GlyRS^{CMT2D}, the quantity of aaRS in disease subjects will be greater than that in healthy subjects.

While the concentration of the capture reagents will generally be determined by the concentration range of interest of the VEGF or aaRS taking any necessary dilution of the biological sample into account, the final concentration of the capture reagents will normally be determined empirically to maximize the sensitivity of the assay over the range of interest. However, as a general guideline, the molar excess is suitably less than about ten-fold of the maximum expected molar concentration of VEGF or aaRS in the biological sample after any appropriate dilution of the sample.

The conditions for incubation of sample and immobilized capture reagent are selected to maximize sensitivity of the assay and to minimize dissociation. In some embodiments, the incubation is accomplished at fairly constant temperatures, ranging from about 0° C. to about 40° C., preferably from about 36 to 38° C. to obtain a less variable, lower coefficient of variant (CV) than at, e.g., room temperature. The time for incubation depends primarily on the temperature, being generally no greater than about 10 hours to avoid an insensitive assay. In some embodiments, the incubation time is from about 0.5 to 3 hours, and more preferably 1.5-3 hours at 36-38° C. to maximize binding of VEGF and/or aaRS to capture reagents. The duration of incubation may be longer if a protease inhibitor is added to prevent proteases in the biological fluid from degrading the VEGF and/or aaRS.

The pH of the incubation mixture can vary, for example be in the range of about 6-9.5, preferably in the range of about 6-7, more preferably about 6.0 to 6.5, and most preferably the pH of the ELISA assay diluent is 6.35±0.1. Acidic pH such as pH 4-5 decreased recovery of VEGF or aaRS. The pH of the incubation buffer is chosen to maintain a significant level of specific binding of the capture reagents to the VEGF being captured. Various buffers may be employed to achieve and maintain the desired pH during this step, including borate, phosphate, carbonate, Tris-HCl or Tris-phosphate, acetate, barbital, and the like. The particular buffer employed is not critical to the methods disclosed herein, but in individual assays one buffer may be preferred over another.

Following incubation of the biological sample (lysed or otherwise treated) on the ELISA plate having immobilized Nrp1 or fragment thereon, the biological sample is separated (preferably by washing) from the immobilized capture reagents to remove uncaptured target molecule (VEGF or aaRS). The solution used for washing is generally a buffer (“washing buffer”) with a pH determined using the considerations and buffers described above for the incubation step, with a preferable pH range of about 6-9. The washing may be done three or more times. The temperature of washing is generally from refrigerator to moderate temperatures, with a constant temperature maintained during the assay period, typically from about 0-40° C., more preferably about 4-30° C. For example, the wash buffer can be placed in ice at 4° C. in a reservoir before the washing, and a plate washer can be utilized for this step. A cross-linking agent or other suitable agent may also be added at this stage to allow the now-bound VEGF or aaRS to be covalently attached to the capture reagents if there is any concern that the captured VEGF or aaRS may dissociate to some extent in the subsequent steps.

Following the wash step, the immobilized capture reagents can be contacted with detectably labeled molecules, such as detection antibodies, for example at a temperature of about 20-40° C., including about 36-38° C., with the exact temperature and time for contacting the two being dependent primarily on the detection means employed. The detectably labeled molecule can be isotopically or non-isotopically labeled. For example, molecules capable of detection include, but are not limited to radioactive isotopes, fluorescers, luminescers, chemilluminescers, enzymes, enzyme substrates, enzyme cofactors, enzyme inhibitors, chromophores, dyes, metal ions, metal sols, ligands (e.g., biotin or haptens), fluorescent nanoparticles, gold nanoparticles, and the like. The term “fluoresce” refers to a substance or a portion thereof that is capable of exhibiting fluorescence in the detectable range such as a fluorophore. Particular examples of labels that can be used include, but are not limited to fluorescein, rhodamine, dansyl, umbelliferon, Texas red, luminol, acridinium esters, NADPH, beta-galactosidase, horseradish peroxidase, glucose oxidase, alkaline phosphatase and urease. The label can also be an epitope tag (e.g., a His-His tag), an antibody or an amplifiable or otherwise detectable oligonucleotide. For example, when 4-methylumbelliferyl-β-galactoside (MUG) and streptavidin-3-galactosidase are used as the means for detection, preferably the contacting is carried out overnight (e.g., about 15-17 hours or more) to amplify the signal to the maximum. The detectable antibody may be a polyclonal or monoclonal antibody. Also, the detectable antibody may be directly detectable, and may be a fluorometric label. The fluorometric label has greater sensitivity to the assay compared to the conventional colorimetric label. In some embodiments, the detectable antibody is biotinylated and the detection means is avidin or streptavidin-β-galactosidase and MUG.

In some embodiments, a molar excess of an antibody with respect to the maximum concentration of VEGF or aaRS expected (as described above) is added to the plate after it is washed. This antibody (which can be directly or indirectly detectable) can be a polyclonal antibody, although any antibody can be employed. The affinity of the antibody must be sufficiently high that small amounts of the VEGF or aaRS can be detected, but not so high that it causes the VEGF or aaRS to be pulled from the capture reagents. The detectably labeled molecule can be removed by washing, as described previously, to remove any unbound molecule.

After binding of the detectably labeled molecule, the level of VEGF or aaRS that is now bound to the capture reagents can be measured using a detection means for the detectably labeled molecule. The measuring step preferably comprises comparing the reaction that occurs as a result of the above three steps with a standard curve to determine the level of VEGF or aaRS compared to a healthy subject.

In some embodiments, the method for diagnosing CMT disease and/or CMT-related neurological diseases in a subject comprises providing a biological sample from a subject having or suspected of having a CMT disease and/or a CMT-related neurological disease. In some embodiments, the biological sample comprises neural tissue, peripheral blood, lymphoblastoid cells, cerebrospinal fluid, muscle tissue, or combinations thereof or other tissue or cellular biological sample having detectable quantities of molecules associated with CMT and/or CMT-related neurological diseases. In some embodiments, cells in the biological sample are lysed, wherein a lysis buffer is provided which is sufficiently strong to dissociate molecules of interest from forming a complex with other molecules. In some embodiments, the biological sample is incubated on an ELISA plate, wherein the ELISA plate is coated with immobilized Nrp1 or a fragment thereof. In some embodiments, the Nrp1 or fragment thereof is a recombinant polypeptide. In some embodiments, the Nrp1 or fragment thereof comprises the b1 domain. In some embodiments, the ELISA plate having Nrp1 immobilized thereon binds to molecules of interest in the biological sample. In some embodiments, the molecules of interest include VEGF or mutated aaRS. In some embodiments, the mutated aaRS is a mutated GlyRS, TyrRS, AlaRS, HisRS, LysRS, or MetRS. In some embodiments, the mutated GlyRS is a missense GlyRS mutant. In some embodiments, the missense GlyRS mutant comprises at least one amino acid substitution selected from the group consisting of P234KY, L129P, E71G, G240R, and combinations thereof. In some embodiments, the mutated GlyRS binds to the immobilized Nrp1 or fragment thereof.

In some embodiments, the ELISA assay is a direct ELISA, a sandwich ELISA, or a competitive ELISA. In some embodiments, an antibody binds to the molecule of interest. In some embodiments, the antibody is detectably labeled, and binding to the molecule of interest creates a detectable signal for determination of the presence and quantity of the molecule of interest. In some embodiments, the antibody is an anti-VEGF or anti-aaRS antibody, including, for example anti-GlyRS, anti-TyrRS, anti-AlaRS, anti-HisRS, anti-LysRS, or antiMetRS antibody.

Some embodiments provide a method for diagnosing CMT and/or CMT-related neurological diseases, where the method comprises providing a biological sample from a patient having or suspected of having CMT and/or CMT-related neurological diseases; lysing cells in the biological sample with a lysis buffer sufficiently strong to dissociate protein interactions; incubating the lysed sample on an ELISA plate having Nrp1 or fragment thereof immobilized thereon; washing the ELISA plate; incubating the ELISA plate with anti-VEGF antibody; and detecting a signal, thereby quantitating the amount of VEGF present in the sample.

The present disclosure further provides methods for diagnosing CMT diseases and/or CMT-related neurological diseases in a subject, wherein the method includes, in some embodiments, providing a biological sample from a subject suspected of having a CMT disease, isolating protein complexes of Neuropilin 1 (Nrp1) from the biological sample, determining the amount of vascular endothelial growth factor (VEGF) in the protein complex, and comparing the amount of VEGF with a reference amount of VEGF from subjects who do not have CMT disease and/or CMT-related neurological diseases, whereby a lower VEGF amount in samples of subjects having or suspected of having a CMT disease and/or a CMT-related neurological disease indicates that the subject suffers from a CMT disease and/or a CMT-related neurological disease. The biological sample can comprise, but is not limited to, neural tissue, peripheral blood, or lymphoblastoid cells, cerebrospinal fluid, muscle tissue or any combination thereof. The method further includes, in some embodiments, lysing cells in the biological sample. In some embodiments, the Nrp1 complex is isolated by immunoprecipitation using an antibody against Nrp1 (an anti-Nrp1 antibody). In some embodiments, the anti-Nrp1 antibody can be a monoclonal or polyclonal antibody. In some embodiments, the anti-Nrp1 antibody binds to the b1 domain of Nrp1. In some embodiment, the anti-Nrp1 antibody binds to one or more of the a1, a2, b1, b2, and c domain of Nrp1. In some embodiments, the anti-Nrp1 antibody binds to the extracellular domain of Nrp1. In some embodiments, the determination of the amount of VEGF in the protein complex is determined by detecting VEGF using an antibody against VEGF (anti-VEGF antibody). In some embodiments, the anti-VEGF antibody is monoclonal or polyclonal. In some embodiments, determining the amount of VEGF comprises dissociating VEGF from Nrp1.

Some embodiments provide a method for ascertaining the presence of aaRS in a subject to diagnose CMT disease and/or CMT-related neurological diseases. The method includes, in some embodiments, isolating protein complexes from a sample and determining the amount of aaRS in the isolated complex as indicative of the presence or absence of a mutant aaRS. In some embodiments, the amount of aaRS is compared to a reference amount of aaRS in subjects that do not have CMT diseases and/or CMT-related neurological diseases.

In some embodiments, the method further includes determining the presence of a mutated aaRS in a biological sample. In some embodiments, the subject suffers from a subtype of CMT referred to as CMT2D, which is caused by dominant mutations in GARS, encoding GlyRS. Mutations in GlyRS (GlyRS^{CMT2D}) alter the conformation of GlyRS, enabling GlyRS^{CMT2D} to bind Nrp1. In some embodiments, the method for ascertaining the presence of GlyRS^{CMT2D} comprises isolating protein complexes comprising Nrp1 from a biological sample and determining the amount of GlyRS in the isolated protein complex as indicative of the presence or absence of GlyRS^{CMT2D} in the subject. In some embodiments, the method comprises isolating protein complexes comprising Nrp1 from a sample from a subject suspected of having a CMT disease and/or a CMT-related neurological disease, determining the amount of GlyRS in the protein complex, and comparing the amount of GlyRS with a reference GlyRS from subjects who do not have CMT diseases, whereby a higher quantity of GlyRS in patients suspected of having CMT is indicative of the presence of mutated GlyRS in the sample. In some embodiments, the mutated GlyRS is GlyRS^{CMT2D}. In some embodiments, the mutated GlyRS is a missense GlyRS mutant. In some embodiments, the missense GlyRS mutant comprises at least one amino acid substitution selected from the group consisting of P234KY, L129P, E71G, G240R, and a combination thereof. In some embodiments, isolating the protein complexes comprising Nrp1 includes isolating whole Nrp1 or a fragment thereof, wherein the fragment comprises the b domains of Nrp1. In some embodiments, isolating the protein complexes comprising Nrp1 or fragments thereof comprises immunoprecipitating the sample with an anti-Nrp1 antibody.

Immunoprecipitation

As defined herein, “immunoprecipitation” comprises the steps of preparing a sample containing the target molecule, such as VEGF or GlyRS^{CMT2D}, adding to the sample an anti-Nrp1 antibody, washing the immunoprecipitate, and detecting the target molecule. Immunoprecipitation can be performed, for example, with an anti-Nrp1 antibody (for example rabbit anti-Nrp1 antibody) and the precipitates are subjected to Western-blot analysis using anti-GlyRS antibody and/or anti-VEGF antibody. The quantities of target molecule are compared to a standard.

Kits for Detection of Mutant aaRS

Also provided herein are kits for the detection of mutant aaRS. In some embodiments, the kit comprises a cell lysis buffer, a solid support coated with a Nrp1 protein or a fragment thereof, and a detectably labeled molecule that specifically binds to an aaRS. As described herein, the label on the detectably labeled molecule can vary, and can be isotopical or non-isotopical. Examples of such kits include, but not limited to, ELISA, immunoprecipitation, or point-of-care (POC) diagnostic kits for the detection and/or quantitation of mutant aaRS and/or VEGF from a biological sample of a subject having or suspected of having a CMT disease. In some embodiments, the kit comprises: a cell lysis buffer; a solid support coated with a capture molecule, wherein the capture molecule specifically binds to Neuropilin 1 (Nrp1) protein or a fragment thereof; and a detectably labeled molecule that specifically binds to vascular endothelial growth factor (VEGF) or a fragment thereof.

POC diagnostics provide reliable, inexpensive, portable, rapid, and simple approaches capable of diagnostic testing. However, POC devices for CMT diseases have not been realized. The lateral flow device, also known as a lateral flow assay (LFA), is one of POC diagnostic tools that is capable of identifying biomarkers in a biological sample. Like most POC devices, LFA devices are minimally invasive, inexpensive, portable, and reliable. Other POC devices capable of detecting biomarkers include the flow through device (FTD).

One common example of a LFA is the common household pregnancy test. LFAs generally involve the use of a labeled antibody deposited at a first position on a solid substrate. Sample is applied to the first position, causing the antibody to dissolve in solution, whereupon the antibody recognizes and binds a first epitope on the analyte in the sample. A complex of analyte and antibody forms and this complex flows along the liquid front from the first location through the solid substrate to a second location, a test line, where immobilized antibodies are located. The immobilized antibody recognizes and binds a second epitope on the analyte, resulting in a high concentration of labeled antibody at the test line. The high concentration of labeled antibody provides a detectable visual signal. Gold nanoparticles are typically used to label the antibodies because they are relatively inexpensive and provide easily observable color indications based on the surface plasmon resonance properties of gold nanoparticles. Generally, this signal provides qualitative information, such as whether or not the analyte is present in the sample.

In some embodiments, a LFA for the detection of VEGF or a mutant aaRS (for example GlyRS^{CMT2D}) from a biological sample of a person having or suspected of having CMT is provided. In some embodiments, mobilizable anti-VEGF or anti-aaRS labeled antibodies are deposited on the LFA device at a first position. Cells in the biological sample are lysed, as described previously, and deposited on the LFA device, whereupon the labelled antibody binds to VEGF and/or aaRS. The antibody-complex migrates along the sample front to a detection line, where Nrp1 or a fragment thereof is immobilized. VEGF and/or mutant aaRS binds to the b1 domain of Nrp1 or a fragment thereof at the detection line, and a signal is detected. The signal is compared with a standard from healthy subject. Numerous iterations and variations of a LFA can be practiced under similar principles.

Methods of Modulating the Effects of aaRS in a Subject Method of Treating CMT Diseases and CMT-Related Neurological Diseases

A patient suffering from a CMT disease and/or CMT-related neurological diseases can be treated with VEGF alone or in combination with other therapies known to treat the disease or condition. As used herein, “therapy” includes but is not limited to a known drug. In addition, VEGF can be combined with a drug associated with an undesirable side effect. By coupling VEGF with such a drug, the effective dosage of the drug with the side effect can be lowered to reduce the probability of the side effect from occurring.

The present disclosure includes methods of treating a patient diagnosed with CMT by the methods described herein with a therapeutically effective amount of VEGF, comprising administering VEGF to the patient such that CMT and/or the CMT-related neurological diseases is ameliorated or reduced. Also disclosed are methods of treating a patient diagnosed with CMT with a therapeutically effective amount of VEGF, comprising administering VEGF to the patient such that the symptoms of CMT are reduced or inhibited. In some embodiments, the VEGF functions by displacing aaRS (e.g., GlyRS) binding to Nrp1, thereby restoring natural Nrp1 signaling. In some embodiments, the method further comprises acquiring knowledge of interaction of Nrp1 with aaRS in a biological sample from the patient.

CMT diseases treatable by the methods disclosed herein include CMT diseases affected by mutations in aaRS that cause a displacement of endogenous VEGF from binding to Nrp1. Such mutations include, for example, G41R mutation in tyrosyl-tRNA synthetase (TyrRS) or P234KY, E71G, L129P, G240R, G526R, or G598A mutations in GlyRS, or combinations thereof. The treatment includes but is not limited to treatment of CMT with VEGF as disclosed herein, alone, in combination with CMT treatments, or in combination with CMT therapy by methods known in the art, such as with physical therapy, with pain management treatments, or with other treatments or therapies in the art.

Pharmaceutical Formulations

The present disclosure provides methods of diagnosis and treatment by administration to a subject of an effective amount of a therapeutic disclosed herein. The subject may be an animal, including but not limited to animals such as cows, pigs, horses, chickens, cats, dogs, etc., and is preferably a mammal, and most preferably human. In a specific embodiment, a non-human mammal is the subject.

The pharmaceutical compositions can comprise an effective amount of VEGF in combination with a pharmaceutically acceptable carrier. The compositions may further comprise other known drugs suitable for the treatment of CMT disease and/or CMT-related neurological diseases. In some embodiments, a therapeutically effective amount of VEGF is an amount of VEGF that is sufficient to partially or completely displace, outcompete, inhibit, or reduce the binding of mutant aaRS to Nrp1, compared to that which would occur in the absence of VEGF treatment. The effective amount (and the manner of administration) can be determined on an individual basis and will be based on a consideration of the subject (size, age, general health), the severity of the condition being treated, the severity of the symptoms to be treated, the result sought, the specific carrier or pharmaceutical formulation being used, the route of administration, and other factors as would be apparent to those of ordinary skill in the art. The effective amount can be determined by one of ordinary skill in the art using techniques as are known in the art. Therapeutically effective amounts of the compounds described herein can be determined using in vitro tests, animal models or other dose-response studies, as are known in the art. VEGF can be used alone or in conjunction with other therapies. The therapeutically effective amount may be reduced when VEGF is used in conjunction with another therapy.

The pharmaceutical compositions may be prepared, packaged, or sold in formulations suitable for intradermal, intravenous, subcutaneous, oral, rectal, vaginal, parenteral, intraperitoneal, topical, pulmonary, intranasal, buccal, ophthalmic, intrathecal, epidural or another route of administration. The compounds may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. For example, the pharmaceutical compositions disclosed herein can be administered locally to a tumor via microinfusion. Further, administration may be by a single dose or a series of doses.

For pharmaceutical uses, the VEGF treatment may be used in combination with a pharmaceutically acceptable carrier, and can optionally include a pharmaceutically acceptable diluent or excipient.

The present disclosure thus also provides pharmaceutical compositions suitable for administration to a subject. The carrier can be a liquid, so that the composition is adapted for parenteral administration, or can be solid, i.e., a tablet or pill formulated for oral administration. Further, the carrier can be in the form of a nebulizable liquid or solid so that the composition is adapted for inhalation. When administered parenterally, the composition should be pyrogen free and in an acceptable parenteral carrier. Active compounds can alternatively be formulated or encapsulated in liposomes, using known methods. Other contemplated formulations include projected nanoparticles and immunologically based formulations.

Liposomes are completely closed lipid bilayer membranes which contain entrapped aqueous volume. Liposomes are vesicles which may be unilamellar (single membrane) or multilamellar (onion-like structures characterized by multiple membrane bilayers, each separated from the next by an aqueous layer). The bilayer is composed of two lipid monolayers having a hydrophobic “tail” region and a hydrophilic “head” region. In the membrane bilayer, the hydrophobic (nonpolar) “tails” of the lipid monolayers orient toward the center of the bilayer, whereas the hydrophilic (polar) “heads” orient toward the aqueous phase.

### EXAMPLES

Some aspects of the embodiments discussed above are disclosed in further detail in the following examples, which are not in any way intended to limit the scope of the present disclosure. Those in the art will appreciate that many other embodiments also fall within the scope of the invention, as it is described herein above and in the claims.

### Experimental Materials and Methods

The following experimental materials and methods were used for Examples 1-5 described below.

Animals

The following strains of mice were used in this study: wild-type C57BL/6J (JAX), P234KY-CMT2D mutant (background includes a mix of C57BL/6, CB6 and CAST), Tg (Hb9:GFP), Tg (ISL^{MN}:GFP-F) (Lewcock et al., 2007), Nrp1 mutant (Gu et al., 2003), TrkB mutant (Xu et al., 2000), Robo1 mutant (Ma et al., 2007), DCC mutant (Fazeli et al., 1997), and Unc5C mutant (Burgess et al., 2006). Both male and female mice were used. All experiments were done in accordance with Institutional Animal Care and Use Committee animal protocols and BSL2+ safety protocols, on animals housed in groups on a 12-h light-dark cycle.

Recombinant GlyRS Expression and Purification

C-terminal His-tagged human GlyRS^{WT} and GlyRS^{CMT2D} proteins were individually cloned into pET21b vector (Novagen) and expressed in Escherichia coli BL21 (DE3) host cells at 25° C. The proteins were purified by Ni-NTA agarose affinity column followed by ion exchange monoQ column and size exclusion column Superdex 200 (GE Healthcare). To prepare non-tagged human GlyRS^{WT} and GlyRS^{CMT2D} proteins, the GlyRS gene was fused with an N-terminal His-SUMO tag, cloned into pET28a vector (Novagen), and expressed as His-SUMO-GlyRS fusion proteins in Escherichia coli BL21 (DE3) cells. The fusion proteins were purified with a Ni-NTA agarose affinity column, and then subjected to homemade Ulp1 protease to remove the His-SUMO tag. The non-tagged GlyRS proteins were separated from the tag by flowing through the Ni-NTA column again.

Hydrogen-Deuterium Exchange (HDX) Analysis

Solution-phase amide HDX was performed with a fully automated system as described previously (Chalmers et al., 2006). Briefly, 4 μL of His-tagged GlyRSP234KY or GlyRSWT was diluted to 20 μL with D₂O-containing HDX buffer to a final concentration of 10 μM, and incubated at 4° C. for 10, 30, 60, 900, and 3,600 seconds. Following on-exchange, unwanted back exchange was minimized by adding 30 μL of 1% TFA in 5M urea to denature the protein (held at 1° C.). Samples were then passed across an immobilized pepsin column (prepared in house) at 50 μL min−1 (0.1% TFA, 15° C.), and the resulting peptides were trapped onto a C8 trap cartridge (Thermo Fisher, Hypersil Gold). Peptides were eluted across a 1 mm×50 mm C18 HPLC column (Hypersil Gold, Thermo Fisher) with a 4-40% CH₃CN gradient and 0.3% formic acid over 5 min at 2° C., and electrosprayed directly into an Orbitrap mass spectrometer (LTQ Orbitrap with ETD, Thermo Fisher). Data were processed with in-house software (Pascal et al., 2012) and visualized with PyMOL (DeLano Scientific). The difference in HDX between GlyRS^{P234KY} and GlyRS^{WT} was calculated by subtracting the average percentage deuterium uptake for GlyRS^{P234KY} from that for GlyRS^{WT} following 10, 30, 60, 300, 900 and 3,600 seconds of on-exchange. The numbers obtained for GlyRS^{P234KY} cannot be directly compared with those for other GlyRS^{CMT2D} mutants from previous studies (He et al., Proc. Natl. Acad. Sci. 108: 12307-12312 (2011)), because this and the precious analysis were carried out in two different laboratories with different instruments and experimental procedures.

Detection of GlyRS Proteins in Cell Cultures

NSC-34 motor neuron cells (Cellutions Biosystems Inc.) and C2C12 mouse adherent myoblasts (From Dr. Ardem Patapoutian's lab at The Scripps Research Institute) were maintained in Dulbecco's modified Eagle's medium (DMEM) supplemented with 10% heat-inactivated fetal bovine serum (FBS) and 1% penicillin (Life Technologies) at 37° C. in humidified incubator containing 5% C02. These cell lines had not been recently authenticated and tested for mycoplasma contamination. Myogenic differentiation of C2C12 myoblasts was induced by substituting the FBS with 2% horse serum. The cells were further cultured in Opti-MEM Reduced Serum Medium (Life Technologies) for 16 h. Brefeldin A (Cell Signaling Technology), GW4869 (Sigma-Aldrich), or monensin (eBioscience) was added to the cell medium for 2 h before the cells and the medium were separated for Western-blot analysis. After removal of cell debris by spinning the medium at 300×g for 10 min, the supernatant was concentrated using Amicon Ultra-4 Centrifugal filter (Millipore). Cells were lysed using cell lysis buffer (ATCC) with added protease inhibitor cocktail (Roche). The following antibodies were used for Western blot analysis: mouse anti-GlyRS (H00002617-B01P, ABNOVA; 1:1000), Rabbit anti-GAPDH (#3683, Cell Signaling Technology; 1:1000), Rabbit anti-vWF (sc-14014, Santa Cruz; 1:50), and Rabbit anti-TSG101 (MABC649, Millipore; 1:1000). To study the effect of CMT2D-causing mutation on GlyRS secretion, constructs overexpressing V5-tagged GlyRS^{P234KY} or GlyRS^{WT} were transfected into COS7 cells using lipofectamine 2000 (Invitrogen). The expression and secretion of GlyRS proteins were detected by Western blot analysis using anti-V5 antibody (R960-25, Invitrogen; 1:5000).

Exosome Purification and Analysis

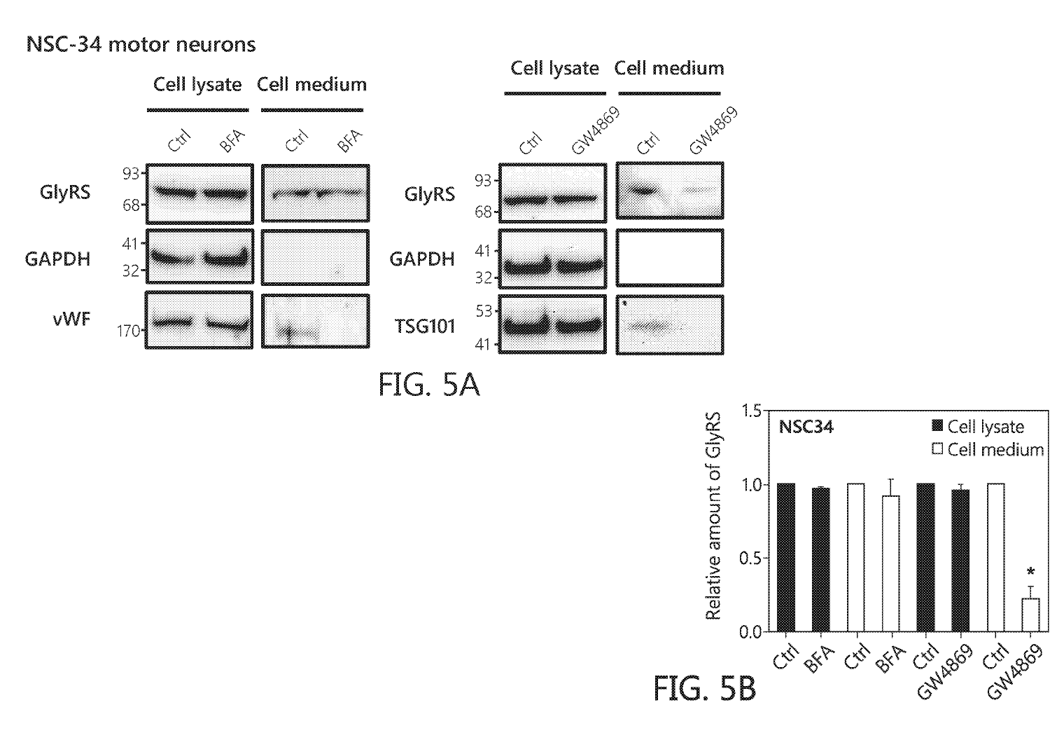

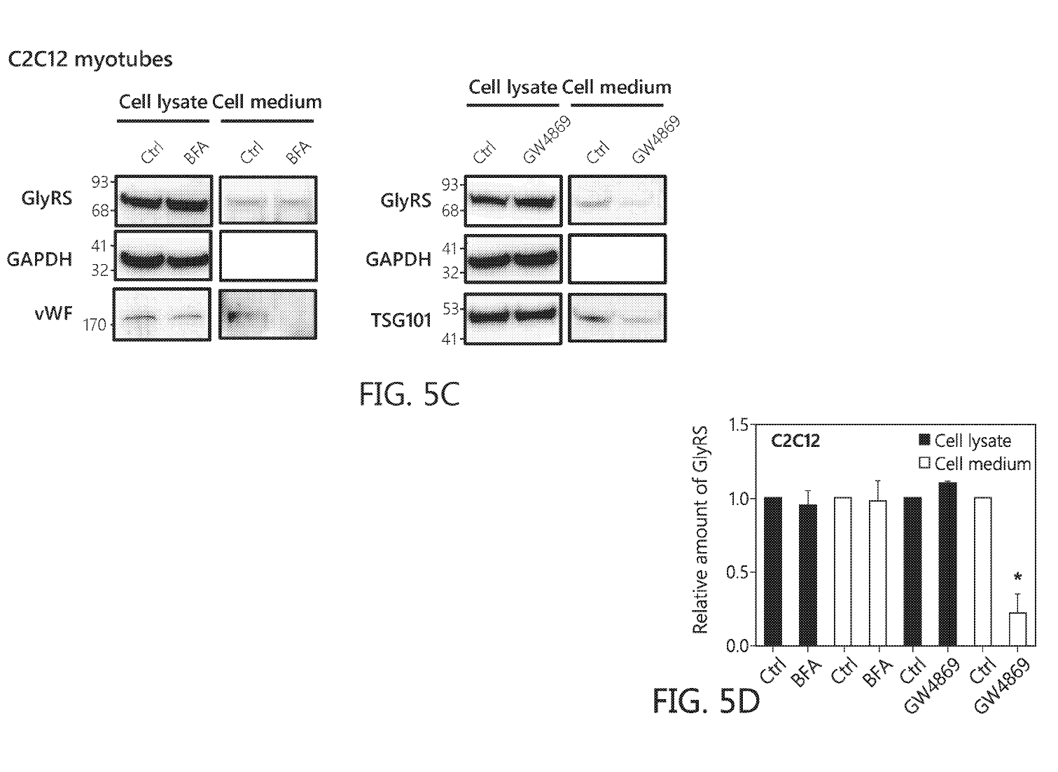

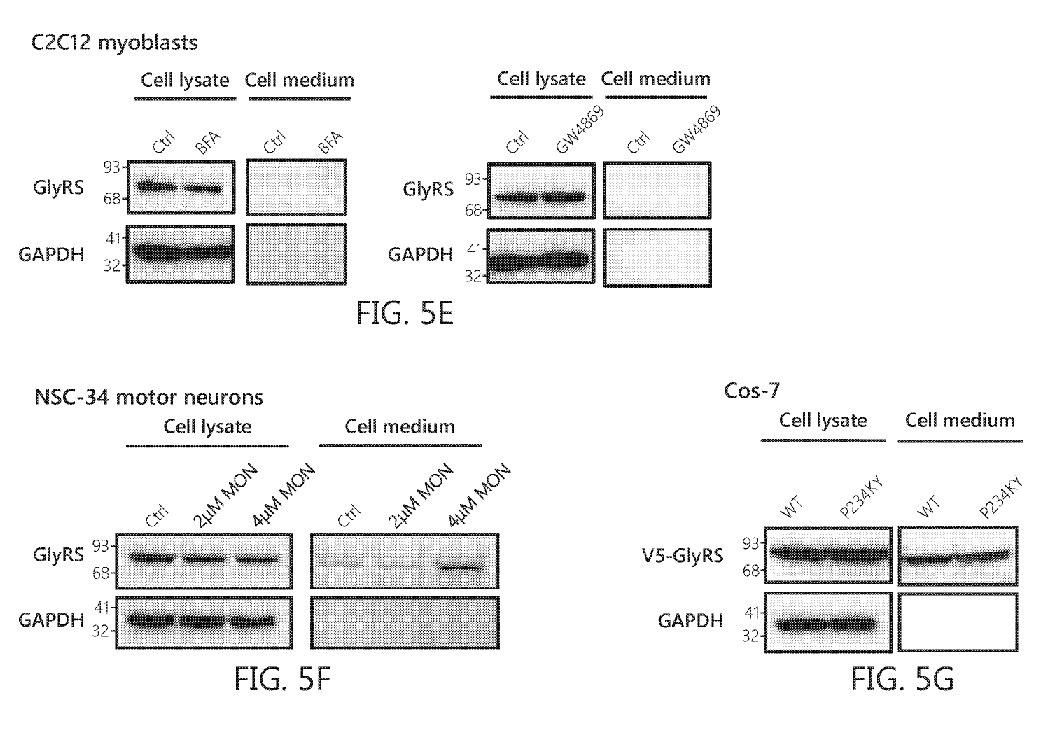

The general idea of exosome purification by differential centrifugation is depicted in FIG. 5A. Supernatants from NSC-34 cell media were subjected to successive centrifugation steps at 4° C.: 1) 200×g for 10 min to eliminate floating cells; 2) 2,000×g for 10 min to discard large dead cells; 3) 10,000×g for 1 h to remove cell debris and cellular organelles such as mitochondria and lysosomes. At each step, the pellet was thrown away and the supernatant was used for the following step. The final supernatant was centrifuged at 100,000×g at 4° C. for 4 h to pellet micro-vesicles that are commonly known as “exosomes”. The final supernatant and the exosome fraction were analyzed by Western blot analysis using antibodies specified above and Rabbit anti-Bip antibody (#3183S, Cell signaling technology; 1:1000).

In Vitro Pull-Down Assay

Recombinant rat Nrp1-Fc, mouse TrkB-Fc, mouse DCC-Fc, rat Robo1-Fc and human Unc5c-Fc extracellular domain-Fc chimeras (R&D systems) were bound to the Protein G beads. Purified non-tagged GlyRS^{WT} and GlyRS^{CMT2D} proteins were individually added to the receptor-immobilized beads and incubated for 1 h at 4° C. After removal of unbound GlyRS proteins, SDS-loading buffer was directly added to the beads to elute the receptor and its bound GlyRS. The amount of GlyRS bound to receptors was analyzed by Western-blot analysis using mouse anti-GlyRS antibodies (H00002617-B01P, ABNOVA; 1:1000).

Co-Immunoprecipitation and Western-Blot Analysis of Protein Expression in Mouse Tissues

The interaction between endogenous GlyRS and Nrp1 proteins was detected by coimmunoprecipitation. Adult mouse neural samples were lysed using RIPA buffer (Cell Signaling Technology) containing 20 mM Tris-HCl (pH 7.5), 150 mM NaCl, 1 mM Na₂EDTA, 1 mM EGTA, 1% NP-40, 1% sodium deoxycholate, 2.5 mM sodium pyrophosphate, 1 mM β-glycerophosphate, 1 mM Na₃VO₄, and 1 μg/mL leupeptin. Immunoprecipitation was performed with rabbit anti-Nrp1 antibody (NBP1 40666, Novus Biologicals; 1:100) and the precipitates were subjected to Western-blot analysis using mouse anti-GlyRS antibody (H00002617-B01P, ABNOVA; 1:1000).

Western-blot was performed to analyze the expression levels of various neuronal proteins in E12.5 WT and CMT2D mutant neural tissues. The following primary antibodies were used: mouse anti-GlyRS (H00002617-B01P, ABNOVA; 1:1000), rabbit anti-Nrp1 (#3725S, Cell Signaling; 1:1000), rabbit anti-VEGFR1 (#36-1100, Life Technologies; 1:1000), rabbit anti-VEGFR2 (#2479S, Cell Signaling; 1:1000), mouse anti-3-actin (#3700, Cell Signaling; 1:1000), mouse anti-DCC (AF5, Abcam; 1:100), rabbit anti-Robo1 (NB100-60458, Novus Biologicals; 1:2000), mouse anti-NF (2H3, DSHB; 1:100), mouse anti-MAP2 (MAB364, Millipore; 1:500), rabbit anti-GAP43 (AB5220, Millipore; 1:1000), and rabbit anti-β-catenin (#9587, Cell signaling technology; 1:1000).

Co-Immunoprecipitation Using CMT2D Patient Samples

Peripheral blood was drawn from CMT2D patients carrying the L129P mutation and control individuals after obtaining their written informed consent. The study complies with the ethical guidelines of the Medical University of Sofia, Bulgaria and University of Antwerp, Belgium. Lymphocytes were isolated on a Ficoll-Paque gradient, transformed with Epstein-Barr virus and incubated at 37° C. for 2 h. After centrifugation, cells were re-suspended in 4 ml RPMI complete medium (Life Technologies) supplemented with 1% phytohaemagglutinin. Cells were seeded on a 24-well plate and incubated at 37° C., 6% C02 for a minimum of 3 days. After establishment, cell lines were cultivated in RPMI1640 medium containing 15% fetal calf serum, 1% sodium pyruvate, 1% 200M L-glutamine and 2% penicillin/streptomycin. The harvested lymphoblastoid cells were lysed using RIPA buffer (Cell Signaling Technology). Immunoprecipitation was performed with rabbit anti-Nrp1 antibody (Novus Biologicals) and rabbit anti-IgG (#2729, Cell Signaling Technology) and the pull-downed samples were subjected to Western-blot analysis using rabbit anti-GlyRS antibody (sc-98614, Santa Cruz Biotechnology, 1:500).

Mapping of GlyRS^{CMT2D} Interaction Domain on Nrp1

The variants of Nrp1 extracellular domain (ECD) include intact ECD (res. Arg23-Asp840), b1b2c domain (res. Phe273-Asp840), ala2 domain (res. Arg23-Asp272), b1b2 domain (res. Phe273-Phe643), c domain (res. Thr589-Asp840), b1 domain (res. Phe273-Asp428) and b2 domain (res. Lys425-Phe643). These variants were designed as chimera proteins containing a 17-residue secretion signal peptide from myeloid cell surface antigen CD33 (gp67) at the N-terminus and a human IgG Fc domain at C-terminus, and were expressed using pcDNA6.0N/V5-His-A vector (Life Technologies). For each Nrp1 variants, 3 μg plasmids were transfected using Lipofactmine 2000 (Life Technologies) into human HEK293 cells in a 6-well plate. 20 h after transfection, MEM media containing secreted Nrp1 variants were collected and incubated with 30 μL Protein A resins. The Nrp1-bound resins were divided equally into two 1.5 mL Eppendorf tubes and incubated with 5 g of recombinant GlyRS^{CMT2D} or GlyRS^{WT} in 1 mL of Washing Buffer (PBS, 5 mM β-ME, 0.2% BSA and 0.05% Triton X-100) for 1 h. Resins were then washed three times with the Washing Buffer and one time with PBS. The bound proteins were eluted with 30 μL of SDS-PAGE sample buffer and subjected to Western-blotting analysis using mouse anti-GlyRS (H00002617-B01P, ABNOVA; 1:1000) and Rabbit anti-His antibodies (RHIS-45P-Z, ICL Lab; 1:10000) to detect GlyRS and the Nrp1 variants, respectively.

The b1 (res. Phe273-Asp428), b2 (res. Lys425-Glu586), and b b2 domain (res. Phe273-Glu586) of Nrp1 fused with an N-terminal GST tag was cloned into the pET28a vector (Novagen), expressed in E. coli BL21(DE3) cells and purified with GST resin (Qiagen). GST or GST-Nrp1 fusion proteins was incubated with 20 μL GST resin and then bind with non-tagged WT or P234KY GlyRS in 1 mL of Washing buffer (1×PBS, 5 mM BME, 0.2% BSA and 0.05% Triton X-100) for 1 h. GST resins were washed three times with Washing buffer and one more time with PBS. The bound proteins were eluted with SDS-PAGE sample buffer, and subjected to Western blotting analysis.

Competition Assay Between VEGF-A165 and GlyRSCMT2D for Nrp1 Binding

In each experiment, 5 μg of GST-b1b2 protein was bound with 15 μL of GST resin in 1 mL Washing Buffer on ice. The competition was tested in both directions. In one direction, 30 nM of P234KY GlyRS^{CMT2D} was added to GST-b1b2 with an increasing concentration of human VEGF-A165 (IBL); in the opposite direction, 30 nM of VEGF-A165 was added with an increasing concentration of P234KY GlyRS^{CMT2D}. After the resins were washed three times with the Washing buffer and one time with PBS, proteins were eluted with SDS-PAGE sample buffer, and analyzed by Western-blot using rabbit anti-VEGF-A (ABS82, Millipore; 1:2000), mouse anti-GlyRS (H00002617-B01P, ABNOVA; 1:1000) and rabbit anti-GST (#2622, Cell Signaling Technology; 1:1000) antibodies.

Immunostaining and Imaging

Immunostaining of NMJs was performed as described in the prior art (Achilli et al., 2009). Cocktails of the following primary antibodies were used to visualize nerves: rabbit anti-NF (AB1991, Millipore; 1:1000), rabbit anti-Synaptophysin (A0010, Dako; 1:2000), and mouse anti-SV2 (DSHB, 1:1000). Secondary antibodies were Alexa-488 or -647 conjugated (Molecular Probes/Invitrogen; 1:1000). Tetramethylrhodamine-conjugated α-bungarotoxin (T-1175, Molecular Probes/Invitrogen; 1:1000) was used to visualize acetylcholine receptors (AChRs) on muscles. The occupancy of NMJs is measured by examining the overlap of the motor nerve terminal with AChRs on the muscle. At least 40 randomly selected NMJs were examined from each of three mutant and three control mice. The flat-mount preparations of hindbrains were performed as previously described in the art (Lewcock et al., 2007). Rabbit anti-Isl1/2 (Ericson et al., 1992) was used to label facial motor nuclei by whole-mount immunostaining. The distance between facial motor nucleas and trigeminal nucleas was measured for each embryo. Each distance was further normalized to relative distance of WT facial motor nucleas. Rabbit anti-VEGF (ab52917, Abcam, 1:200) was used to determine the expression of VEGF in muscle fibers.

Bright field and fluorescence images of whole embryos were obtained using a 0.8×objective on Zeiss Lumar.V12 fluorescence stereomicroscope. Confocal images were obtained using 10× and 20× objectives on Olympus Fluoview 1000 confocal microscope.

Hindlimb Extension Test

Mice were suspended by the tail and the extent of hindlimb extension was observed over 10 s. A score of 2 corresponded to a normal extension reflex in hindlimbs with splaying of toes. A score of 1 corresponded to clenching of hindlimbs to the body with partial splaying of toes. A score of 0 corresponded to clasping hindlimbs with curled toes. Three tests were performed for each mouse with 5-s intervals. A score of 1.5 or 0.5 corresponded to behaviors between 2 and 1, or between 1 and 0, respectively.

Footprint Test

Blue ink was applied to the hind paws of each mouse and the animal was placed in a narrow alley (9×80×25 cm) with the floor covered with white paper. A home cage was placed at the end of the alley for the animal to walk to while leaving its footprints on the paper. Stride length was assessed by measuring the average distances of at least three consecutive steps on each side.

Inclined Plane Test

Hindlimb strength was assessed at postnatal 4 weeks using the inclined plane test. Briefly, animals were placed on an inclined plane, and the angle of incline was gradually increased starting from 15°. The maximum angle at which the animal could maintain its position for 5 sec constituted the inclined plane score. The test was performed 3 times for each mouse.

Rotarod Test

Motor coordination was assessed with a rotarod apparatus (Economex, Columbus Instruments). The mice were first placed on the stationary rod (0 rpm) to acclimate them to the apparatus, followed by a trial at a rotation speed of 1 rpm for 3 min or until a fall occurred. For testing, the rotation of the rotarod was accelerated from 0 rpm with an accelerating rate (0.1 rpm/min). Latency of each mouse to fall was monitored for three consecutive trials and the intra-trial interval for each animal was about 20 min. The average time of three trials was used as a measure of motor performance.

Virus Preparation and Injection

The cDNAs encoding GDNF, VEGF-A121, or VEGF-A165 were cloned into lentiviral vector (p156RRLsinPPTCMVGFPPRE) between BamHI and SalI sites. All lentiviruses were produced by GT3 core facility at Salk Institute with a titer of 1×10¹²−2×10¹³ genome copies/mL. Injections were performed at P5 (±1 day) after anesthetizing pups on ice. Multiple injections (n≥8) of virus (5 μL for each limb) into a variety of hindlimb muscles were performed with a Hamilton syringe. Based on the expression pattern of GFP reporter, the lentiviruses mainly infect muscle fibers.

Nerve Histology and Imaging

Mouse sciatic nerves were dissected and fixed in 2.5% glutaraldehyde in 0.1 M phosphate buffer. Nerve samples were then osmicated, dehydrated, and embedded in araldite resin. Transverse nerve sections (1 μm) were cut on a Leica RM2065 microtome and stained with methylene blue Azure II. Images were collected on a Leica DMR microscope or an Olympus BX61 microscope. Axon numbers were determined from two non-overlapping fields (50×50 μm) from each of three mutant and three control nerve samples. Axon diameters were measured by Image J.

Statistics

All graphs and data generated in this study were analyzed using GraphPad Prism 6.0 Software (MacKiev) or Excel (Microsoft). Two-tailed unpaired t-tests with Welch's correction using parametric distribution, two-tailed Mann-Whitney test using unparametric distribution, or twotailed paired Wilcoxon test using unparametric distribution were performed to measure differences from at least three independent biological replicates. P<0.05 was considered significant. These tests do not require similar variance of the data between the groups that are being statistically compared. The normality of the data was determined by D'Agostino-Pearson omnibus test and Kolmogorov-Smirnov test. No statistical methods were used to predetermine sample sizes, but the sample sizes are similar to those generally used in the field.

For all animal studies, analyses were performed on approximately equal numbers of male and female mice selected randomly from populations, and no sex-specific differences in the disease progression were identified. All behavioral experiments were performed in double-blind fashion, and stressed animals were excluded from the analysis.

### Example 1

### CMT2D Mutations Cause Neomorphic Structural Openings at the Dimer Interface of GlyRS

This example demonstrates that mutations in GlyRS cause overall structural opening of GlyRS.

CMT diseases are a group of inherited disorders that specifically affect the peripheral nervous system and are characterized by progressive weakness and atrophy in the hands and feet. GARS, encoding glycyl-tRNA synthetase (GlyRS) mutations cause a dominant axonal form of CMT (CMT2D) (Antonellis et al., Am. J. Hum. Genet. 72:1293-1299 (2011)). The canonical function of this evolutionarily ancient cytoplasmic enzyme is to catalyze the ligation of glycine to the 3′-end of its cognate tRNA as the first step of protein synthesis. Interestingly, emerging evidence reveals that GlyRS in multi-cellular organisms, like several other tRNA synthease family members, has acquired the ability to be secreted from cells and as an extracellular protein can influence cell signaling (Wakasugi et al., Science 284:147-151 (1999)).

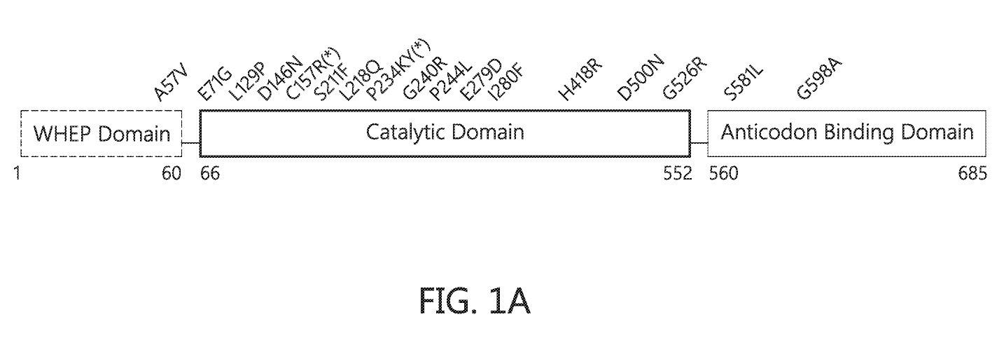

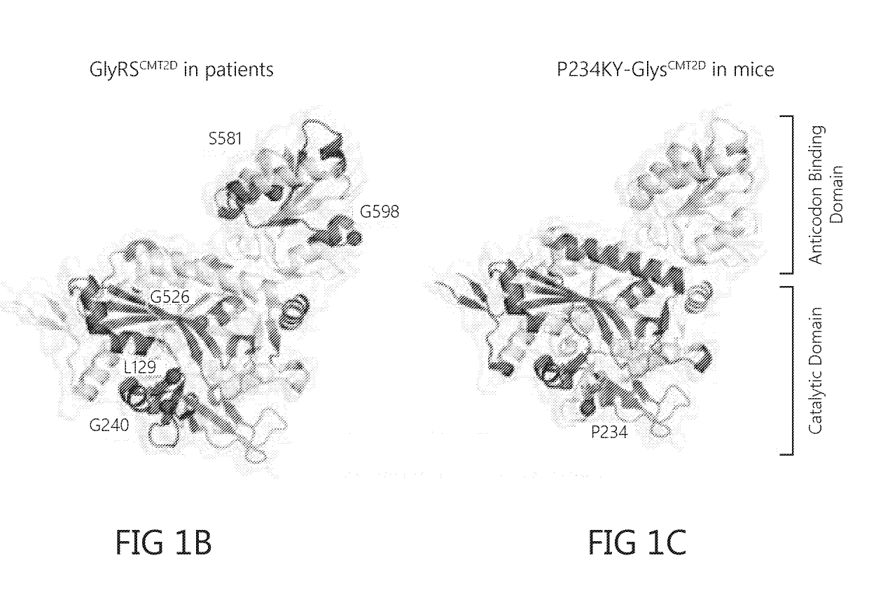

More than a dozen missense GARS mutations (GlyRS^{CMT2D}) have been found in CMT2D patients with varying degrees of genetic evidence for disease-association (Lee et al., J. Peripher. Nerv. Syst. 17:418-421 (2012); Motley et al., Trends Neurosci. 33:59-66 (2010); Kawakami et al., Rinsho Shikeigaku 54:911-915 (2014); Sun et al., Neurol. Res. 37:782-787 (2015)). Among them, three mutations (E71G, L129P and G240R) are the most tightly linked to the disease (FIG. 1A) (Motley et al. Trends Neurosci. 33:59-66 (2010)). Spontaneous and ENU-induced missense mutations in mouse Gars also cause CMT2D-like phenotypes (FIG. 1A) (Achilli et al., Dis. Model Mech. 2:359-373 (2009); Seburn et al., Neuron 51:715-726 (2006)). These dominant mutations are found throughout the primary sequence of GlyRS, with some affecting the aminoacylation enzyme activity whereas others do not (Motley et al. Trends Neurosci. 33:59-66 (2010)). Mice with a heterozygous deletion of the Gars gene and a 50% reduction in glycyl-tRNA synthetase activity are normal (Seburn et al., Neuron 51:715-726 (2006)). Furthermore, overexpression of the wild-type GlyRS (GlyRS^{WT}) in a CMT2D-disease mouse fails to rescue the neuropathy (Motley et al., PLoS Genet. 7:e1002399 (2011). These genetic experiments indicate that CMT2D may arise from an abnormal activity gained by GlyRS^{CMT2D} rather than a general defect in tRNA aminoacylation as initially suspected.

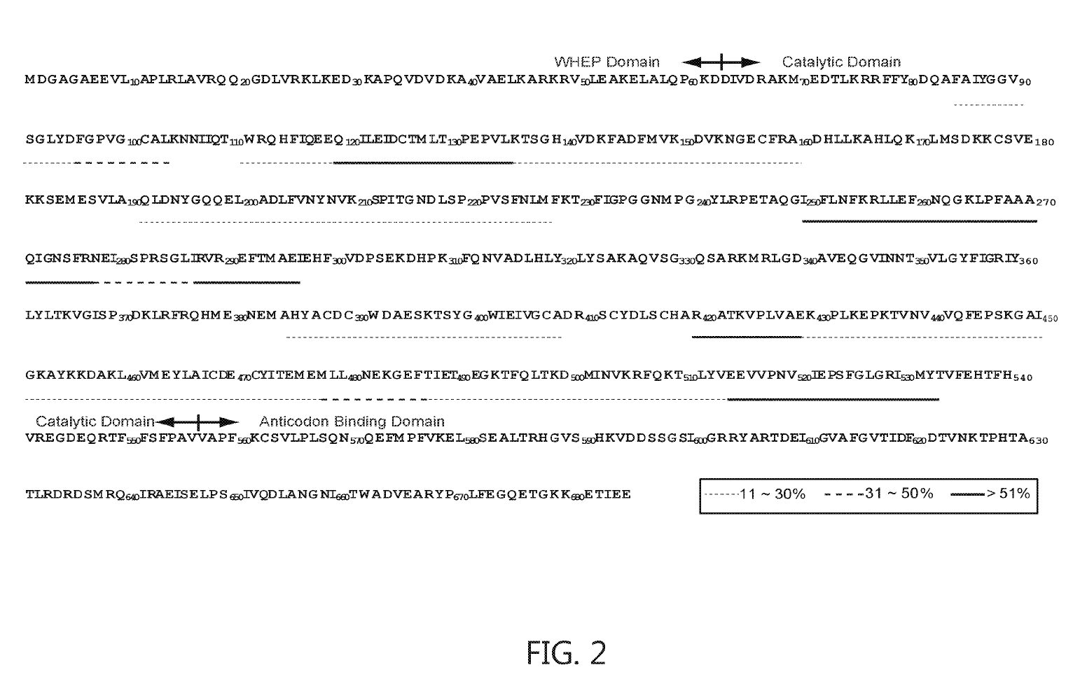

Like most class II tRNA synthetases, GlyRS functions as a dimer for aminoacylation. Interestingly, despite being dispersed in three separate domains of GlyRS, all known CMT2D causing mutations are located near the dimer interface in the GlyRS crystal structure 17 (FIGS. 1B and 1C). Five different human mutations associated with CMT2D caused a conformational opening in GlyRS that exposes new protein surfaces to solution (FIG. 1B) (He et al., PNAS 108:12307-12312 (2011)). To test if this conformational change also occurs in P234KY-GlyR^{SCMT2D} linked to CMT-like phenotypes in mice, hydrogen-deuterium exchange labeling was performed on P234KY-GlyRS. This mutation opens new surfaces of the GlyRS protein to solution (FIGS. 1B and 1C, and FIG. 2). Thus, many of the CMT2D mutants share the abnormal opening.

### Example 2

### Mutant aaRS Bind Nrp1

This example demonstrates that aberrant interactions between various mutant aaRS and Nrp1.

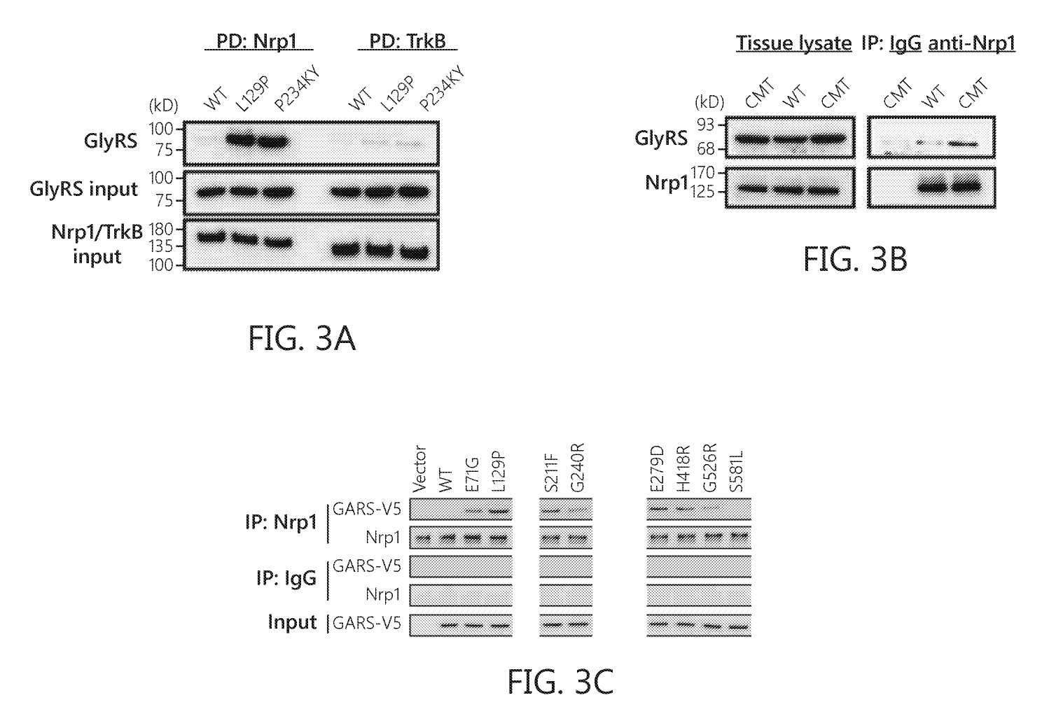

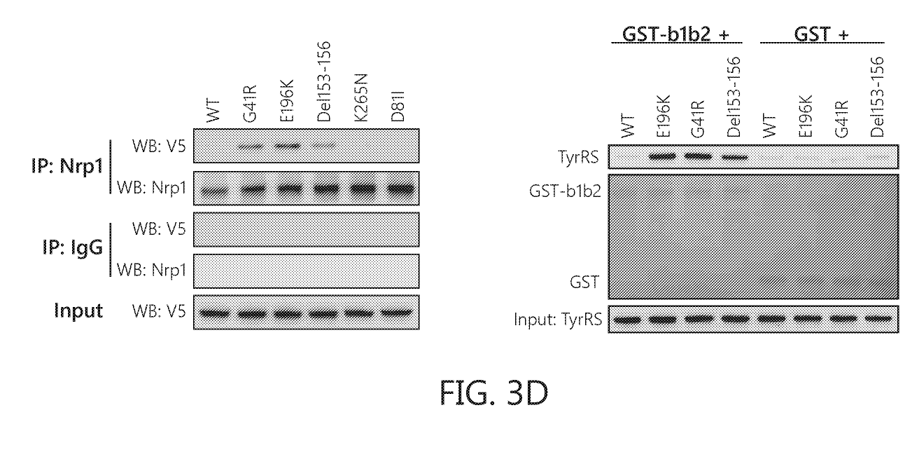

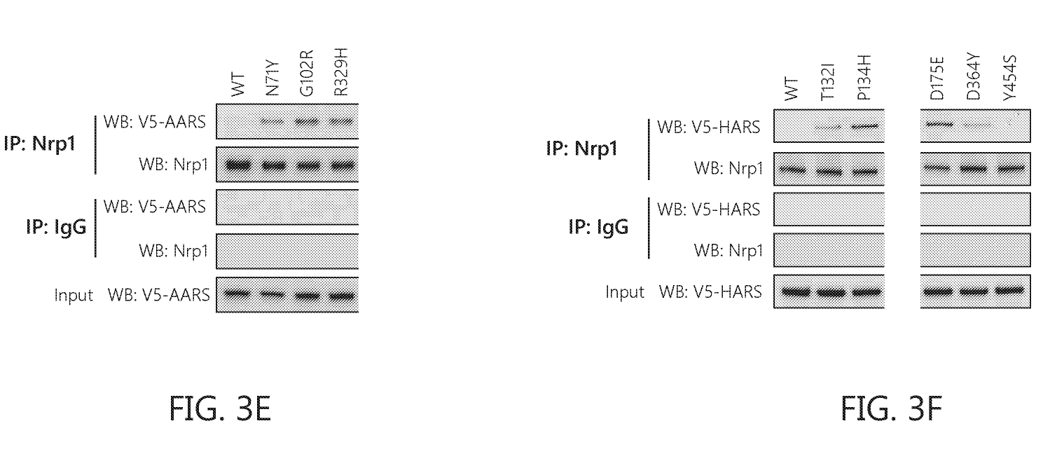

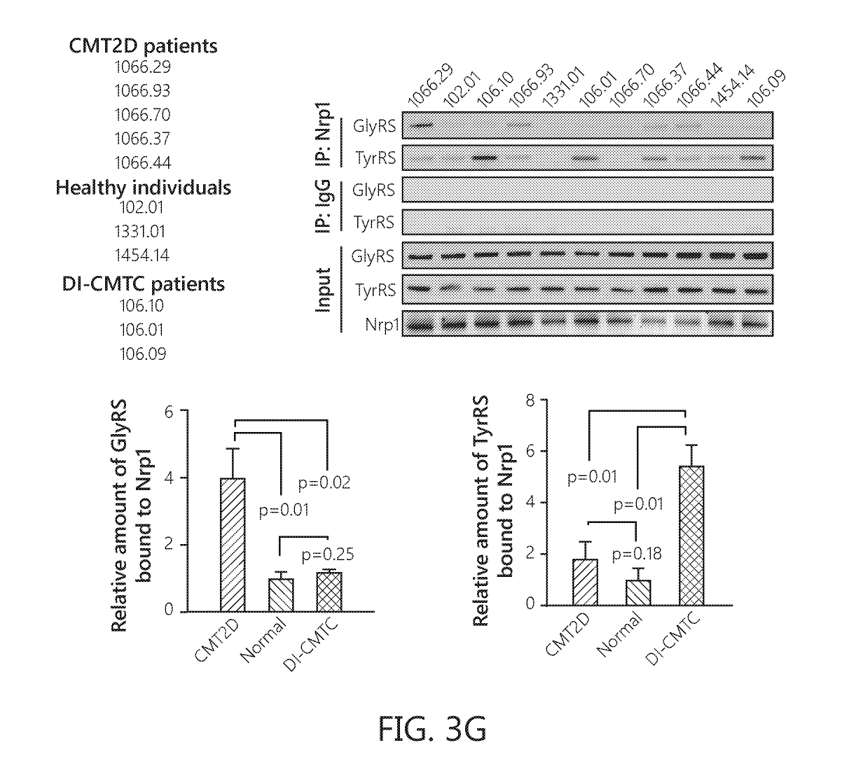

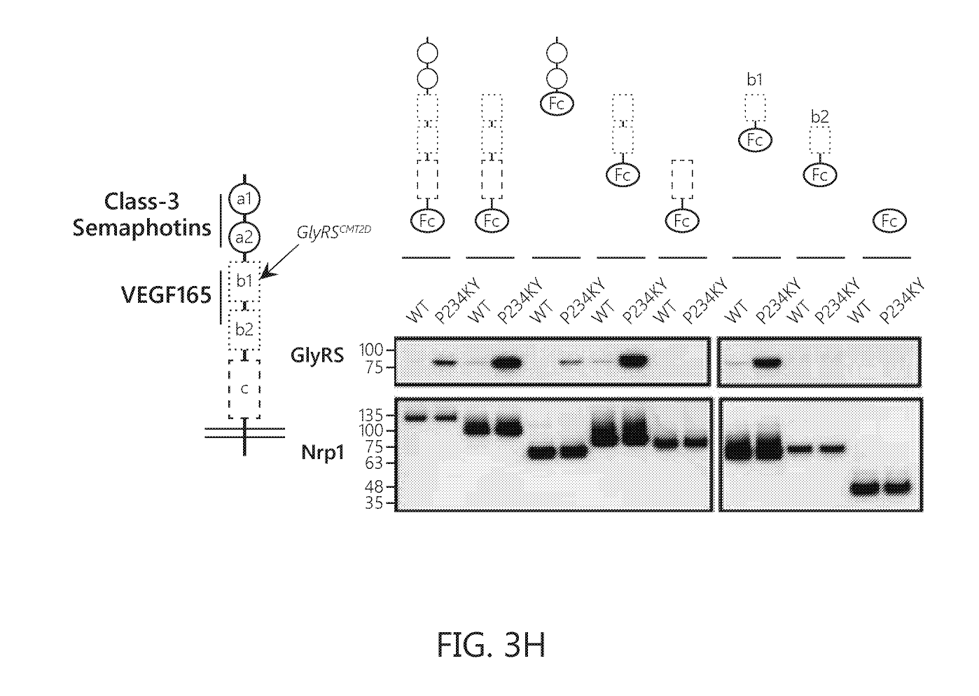

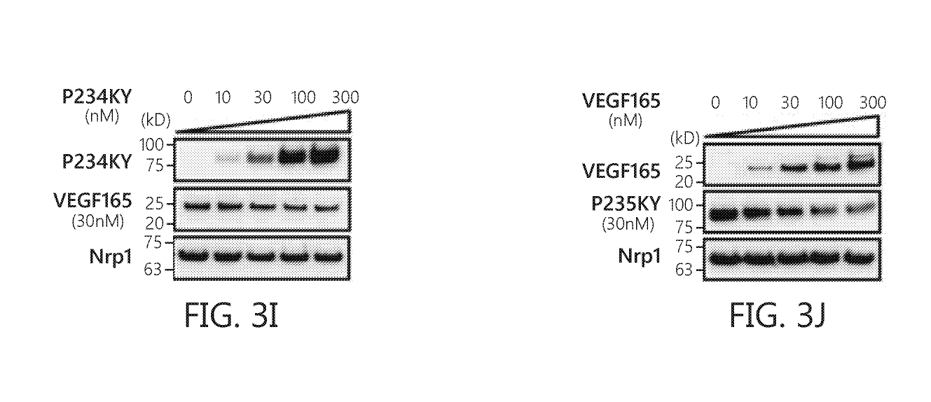

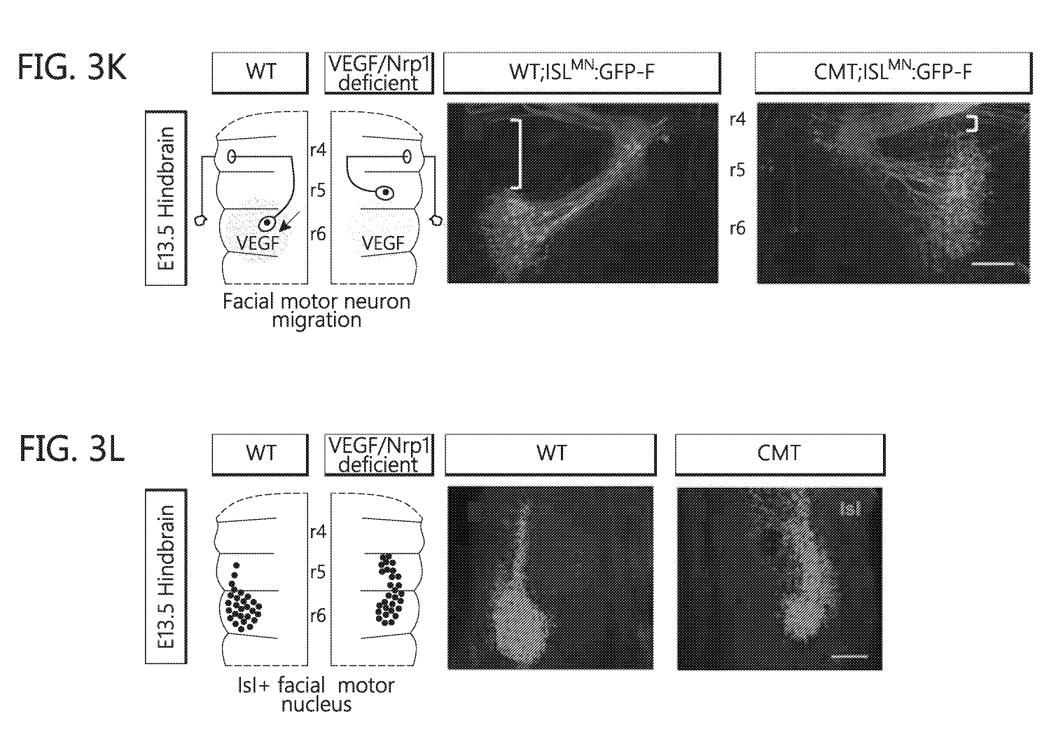

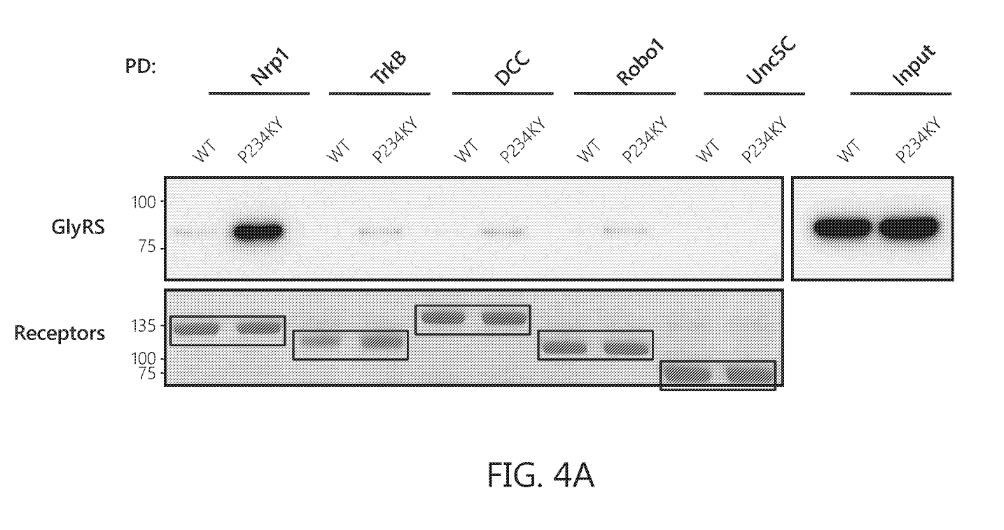

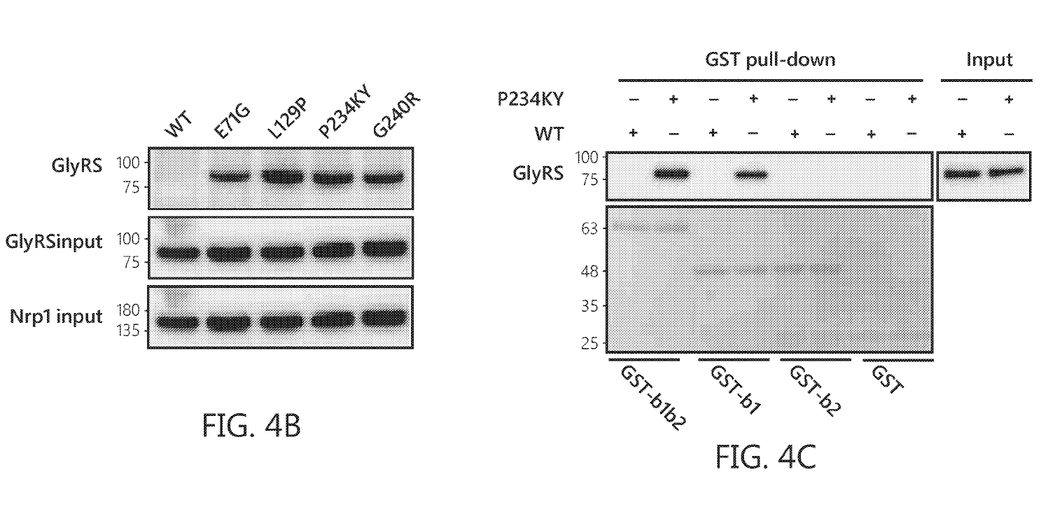

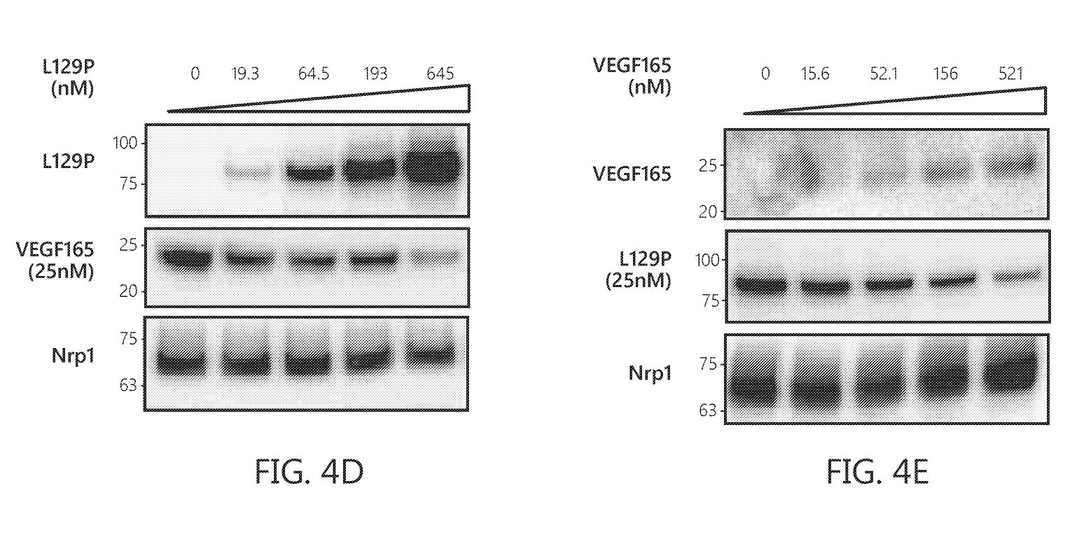

The new surfaces exposed by mutations in GlyRS^{CMT2D} can result in neomorphic protein-interactions. Binding partners unique to GlyRS^{CMT2D} were determined. A candidate-protein screen by in vitro protein pull-down assays was performed. Because motor neurons are the most frequently affected neuronal type in CMT2D (Antonellis et al., Am. J. Hum. Genet. 72:1293-1299 (2011); (Del Bo et al., Neurology 66:752-754 (2006); Dubourg et al., Neurology 66:1721-1726 (2006)), the initial screen focused on molecules that are highly expressed by motor neurons and that have been linked to motor neuron diseases/defects. Strong binding between the receptor Nrp1 and several GlyRS^{CMT2D} mutants was detected, including P234KY and the three (E71G, L129P, and G240R) with the strongest link to CMT2D in patients (FIG. 3A and FIGS. 4A and 4B) (Motley et al. Trends Neurosci. 33:59-66 (2010)). In contrast, GlyRS^{WT} did not bind Nrp1 strongly, and GlyRS^{CMT2D} failed to bind to other motor neuron proteins such as TrkB, DCC, Robo1, and Unc5C (FIG. 3A and FIGS. 4A and 4B). To confirm that this binding-specificity occurs in vivo, immunoprecipitations were performed using neural tissues from WT and P234KY-CMT2D mouse littermates. Anti-Nrp1 antibodies coprecipitated significantly more GlyRS from CMT2D mice than WT controls (FIG. 3B), indicating that P234KY-GlyRS^{CMT2D} binds to Nrp1 in vivo. Aberrant GlyRS-Nrp1 interaction is significantly stronger in CMT2D patients than in healthy individuals (FIG. 3C). This result is consistent with previous data using purified L129P GlyRS protein (FIG. 3A and FIG. 2B), and further demonstrates that the aberrant Nrp1-binding activity of GlyRS^{CMT2D} applies to more than the specific mutation (P234KY). To quantify these interactions, biolayer interferometry (BLI) and a biosensor with immobilized Nrp1 on the surface was used. GlyRS^{WT} binding was undetectable at 1 μM; whereas GlyRS^{CMT2D} bound significantly stronger with a K_{d} of 29.8±6.3 nM for L129P and 208.7±53.8 nM for P234KY.

To address whether the proposed mechanism is applicable to other ARS-associated CMT diseases, DI-CMTC patient samples were obtained, containing G41R mutation in tyrosyl-tRNA synthetase (TyrRS). Remarkably, aberrant interaction between Nrp1 and TyrRS were detected from DI-CMTC patients (FIG. 3D). This aberrant interaction between TyrRS and Nrp1 detected in DI-CMTC patient samples is further confirmed by co-immunoprecipitation in motor neuron NSC-34 cells (FIG. 3D, left) and by GST pull down using purified proteins (FIG. 3D, right). Moreover, the aberrant TyrRS-Nrp1 interaction is not unique to the G41R mutation, but also applies to other TyrRS mutations linked to DI-CMTC (E196K and the deletion mutation Del153-156) (FIG. 3D). In both experiments, G41R and E196K lead to a stronger TyrRS-Nrp1 interaction than Del153-156 (FIG. 3D). It is important to note that G41R and E196K mutations segregate with DI-CMTC in multigenerational families, while Del153-156 is a de novo mutation found in a single DI-CMTC patient (Jordanova et al., Nat. Genet. 38:197-202 (2006)). Therefore, like CMT2D, the strength of the aberrant interaction with Nrp1 also correlates with the strength of disease association for DI-CMTC.

Various other mutant aaRS known to be associated with CMT diseases were also examined. Aberrant Nrp1 interaction was observed, detected by co-immunoprecipitation in NSC-34 cells, with all of the published CMT2N AlaRS mutations tested (FIG. 3E). As shown in FIG. 3F, abberant Nrp1 interaction are also present with all of the published CMT2W HisRS mutations, detected by co-immunoprecipitation in NSC-34 cells. In FIG. 3F, Y454S mutation, which is linked to Usher syndrome, a disease different from CMT, was used as a negative control. FIG. 3G shows the results of a co-immunoprecipitation assay that detected significantly more aberrant GlyRS-Nrp1 interaction in lymphocytes from CMT2D patients carrying the GlyRS L129P mutation (n=5) than from DI-CMTC patients carrying TyrRS G41R mutation (n=3) and from healthy individuals. Similarly, significantly more averrant TyrRS-Nrp1 interaction was detected in lymphocytes from DI-CMTC patients carrying TyrRS g41R mutation (n=3) than from CMT2D patients carrying the GlyRS L129P mutation (n=5) and from healthy individuals. Moreover, within the five CMT2D patients carrying the GlyRS L129P mutation from the same family, the strength of the aberrant GlyRS-Nrp1 interaction seems to correlate with the severity of the CMT2D symptoms. For example, patient 1066.29 has the most severe CMT2D symptoms and also the strongest GlyRS-Nrp1 interaction, while patient 1066.70 has the least severe symptoms and the weakest GlyRS-Nrp1 interaction. These results show that aberrant aaRS-Nrp1 interaction can be used not only to determine the CMT pathogenicity of a tRNA synthetase mutation, but also as a companion diagnostic assay to select patients that are most suitable for targeting the aberrant interaction as a potential therapeutic.

Taken together, the data show that aberrant Nrp1 interactions with conformationally-altered mutant proteins is a common mechanism for different types of tRNA synthetase-linked CMT.

### Example 3

### Interference of VEGF-Nrp1 Interaction by GlyRS^{CMT2D} and Mapping of the Binding Site of GlyRS^{CMT2D} to Nrp1

This example shows that GlyRS^{CMT2D} antagonizes VEGF-Nrp1 interaction and the site where GlyRS^{CMT2D} binds to Nrp1 using pull-down assays with domain-deletion constructs.

Removal of the extracellular a and c domains of Nrp1 did not alter GlyRS^{CMT2D} binding, whereas the extracellular Nrp1-b1 domain alone was sufficient to bind P234KY-GlyRS^{CMT2D} (FIG. 3H and FIG. 4C). Because the b1 domain is the binding site of VEGF-A165, this finding raised the possibility that GlyRS^{CMT2D} might influence the binding of VEGF-A165 to this region of Nrp1. Using pull-down assays, increasing concentrations of P234KY or L129P GlyRS^{CMT2D} were found to compete with VEGF-A165 binding to the b domains of Nrp1 (FIG. 3I and FIG. 4D). Conversely, increasing levels of VEGFA165 displaced P234KY or L129P-GlyRS^{CMT2D} from the b domains (FIG. 3J and FIG. 4D).

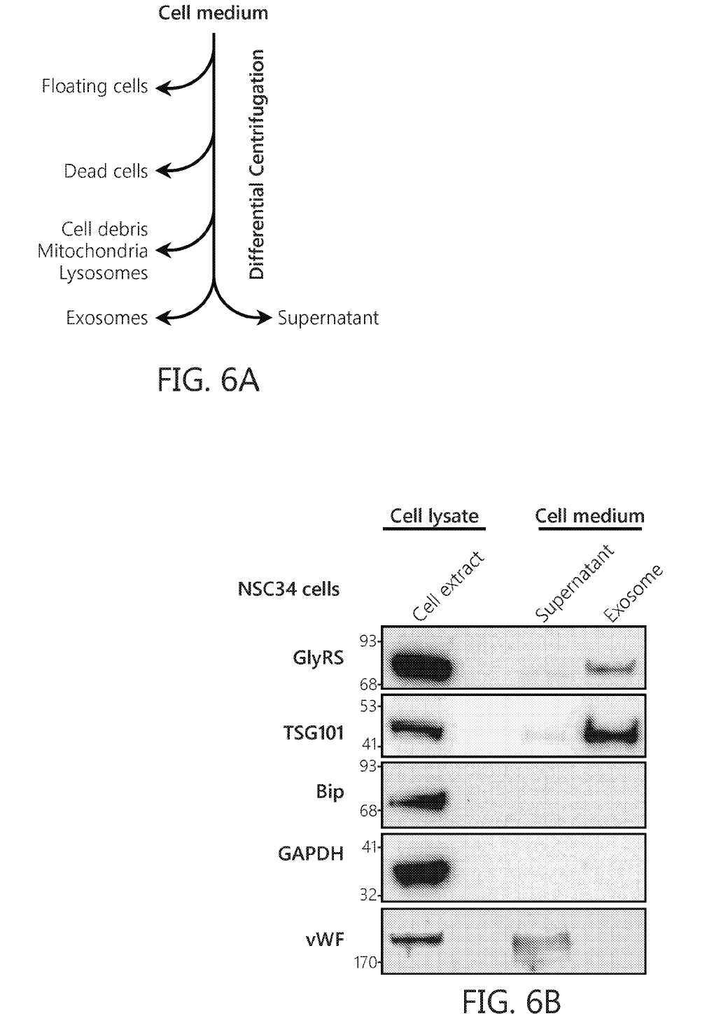

GlyRS protein in the extracellular environment was studied. Recent studies of GlyRS^{WT} detected secretion from immune cells (Park et al., PNAS 109:E640-647 (2012)). The GlyRS^{WT} was examined to determine whether it is released by cell types relevant to the peripheral nervous system and motor function. Indeed, endogenous GlyRS^{WT} was detected in the culture media of mouse motor neuron and differentiated myotube cell lines, but not of undifferentiated myoblasts (FIGS. 5A-5E). Secreted GlyRS^{WT} was enriched from extracellular sources using procedures that concentrate micro-vesicles (30-100 nm, “exosomes”) (FIGS. 6A and 6B). Extracellular levels of GlyRS^{WT} were diminished by application of the exosome-pathway inhibitor GW4869 and enhanced by exosome-pathway activators monensin (FIGS. 5A-5F). It was investigated whether CMT2D-causing mutations affect the secretion of GlyRS^{CMT2D}, and determined that P234KY-GlyRS^{CMT2D} was detected at levels similar to GlyRS^{WT} in the media of transfected cells (FIG. 5G).

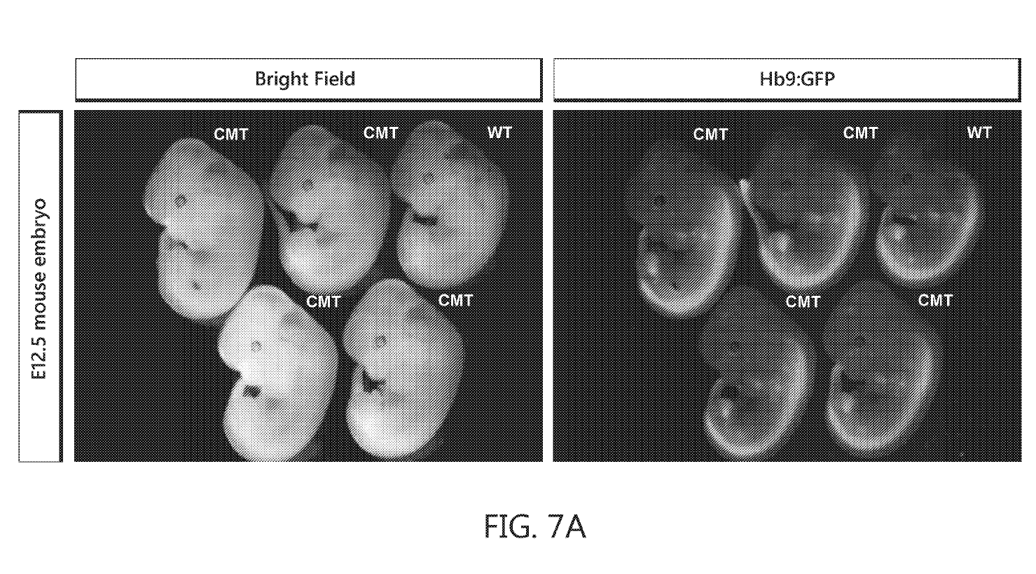

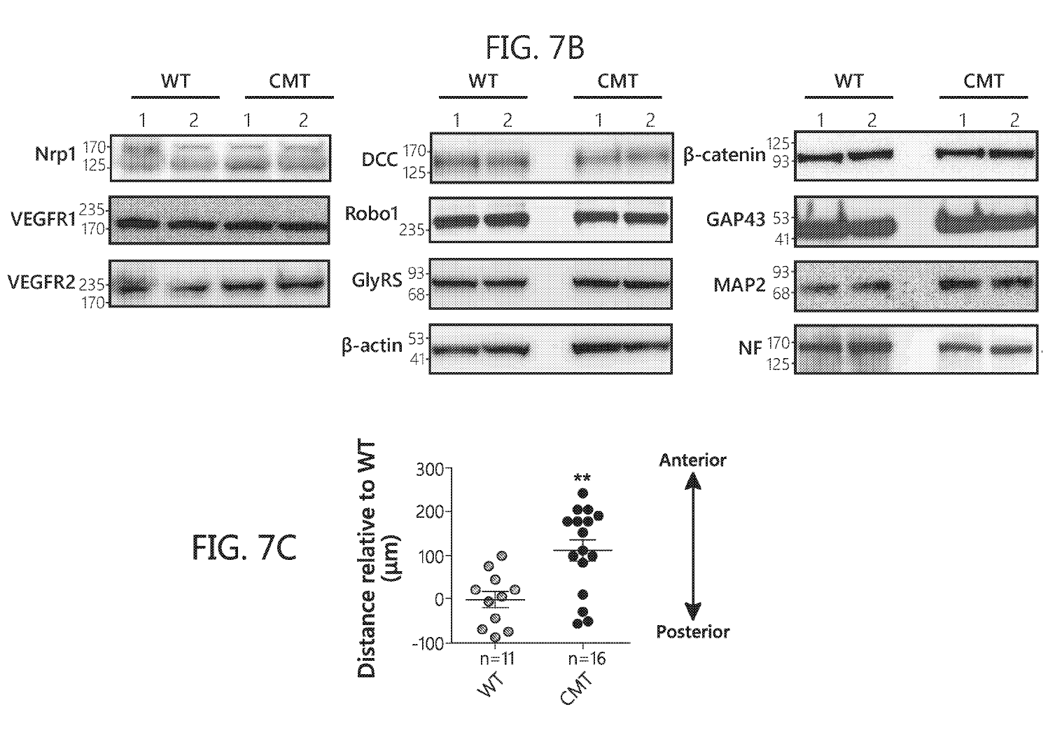

Nrp1 is a well-established receptor needed for motor neuron axon guidance and cell body migration (Schwarz et al, Genes Dev. 18:2822-2834 (2004); Huber et al. Neuron 48:949-964 (2005)). The competition of GlyRS^{CMT2D} and VEGF-A165 for access to Nrp1 as disclosed herein, raised the possibility that CMT2D-mice may phenocopy some features of VEGF-A164 (the mouse equivalent of human VEGF-A165) and Nrp1 mutant mice (Schwarz et al, Genes Dev. 18:2822-2834 (2004)). VEGF/Nrp1 signaling is necessary for the caudal migration of facial motor neurons from rhombomere (r) 4 to r6 during embryonic development (FIGS. 3K and 3L). This provides an excellent in vivo assay to examine the effect of GlyRS^{CMT2D} on a well-characterized system known to depend on VEGF/Nrp1 signaling. To track facial motor neuron migration, CMT2D mice were crossed to transgenic ISL^{MN}:GFP-F reporter animals that selectively label facial motor neurons (Song et al., Development 133:4945-4955 (2006), Lewcock et al., Neuron 56:604-620 (2007)). CMT2D mutant embryos developed at a normal rate, appeared overtly normal based on their overall morphology, and the expression levels of a variety of neuronal proteins were comparable to controls (FIGS. 7A and 7B). However, GFP-labeled facial motor neuron somata were found in ectopic anterior rhombomere locations in E13.5 GlyRS^{CMT2D} mutant embryos and manifested as an elongated stream across multiple rhombomeres (FIG. 3K). In contrast, most facial cells had completed their caudal migration to r6 in littermate controls at this stage (FIG. 3K). This observation was confirmed by immunostaining with the LIM homeodomain transcription factor Isl that is selectively expressed in the nuclei of facial branchiomotor neurons (FIG. 3L and FIG. 7C). This facial motor neuron migration defect closely resembles the phenotypes of Nrp1-null and VEGF-A164-null mice as previously reported (FIG. 3K) (Schwarz et al, Genes Dev. 18:2822-2834 (2004)). Taken together, the protein binding studies and embryological defects associated with GlyRS^{CMT2D} suggested that GlyRS^{CMT2D} inhibits VEGF/Nrp1 signaling.

### Example 4

### Nrp1 is a Genetic Modifier of CMT2D

This example demonstrates that Nrp1 is an important genetic modifier of CMT2D pathology and that GlyRS^{CMT2D} antagonizes normal Nrp1-signaling rather than activating aberrant signaling.

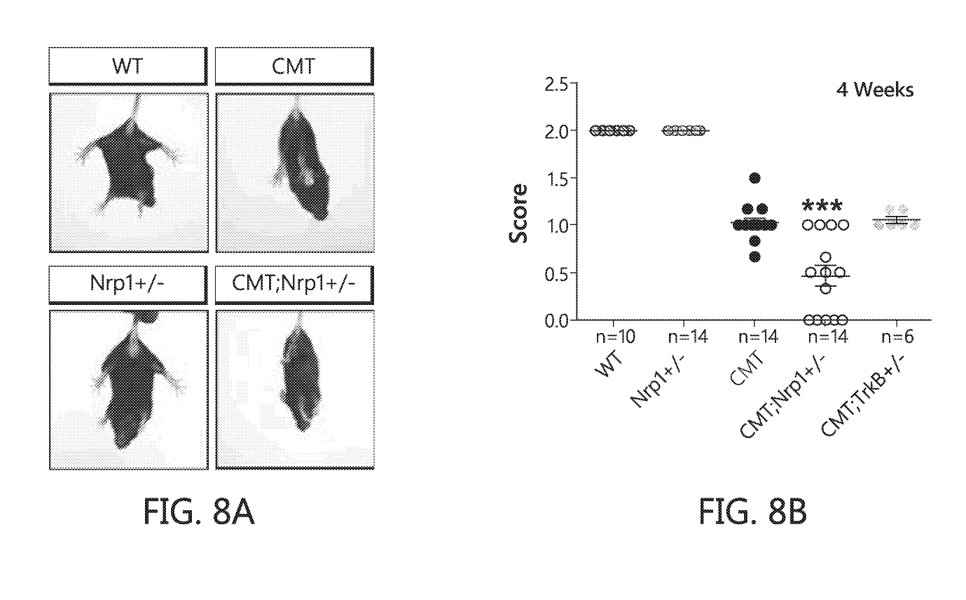

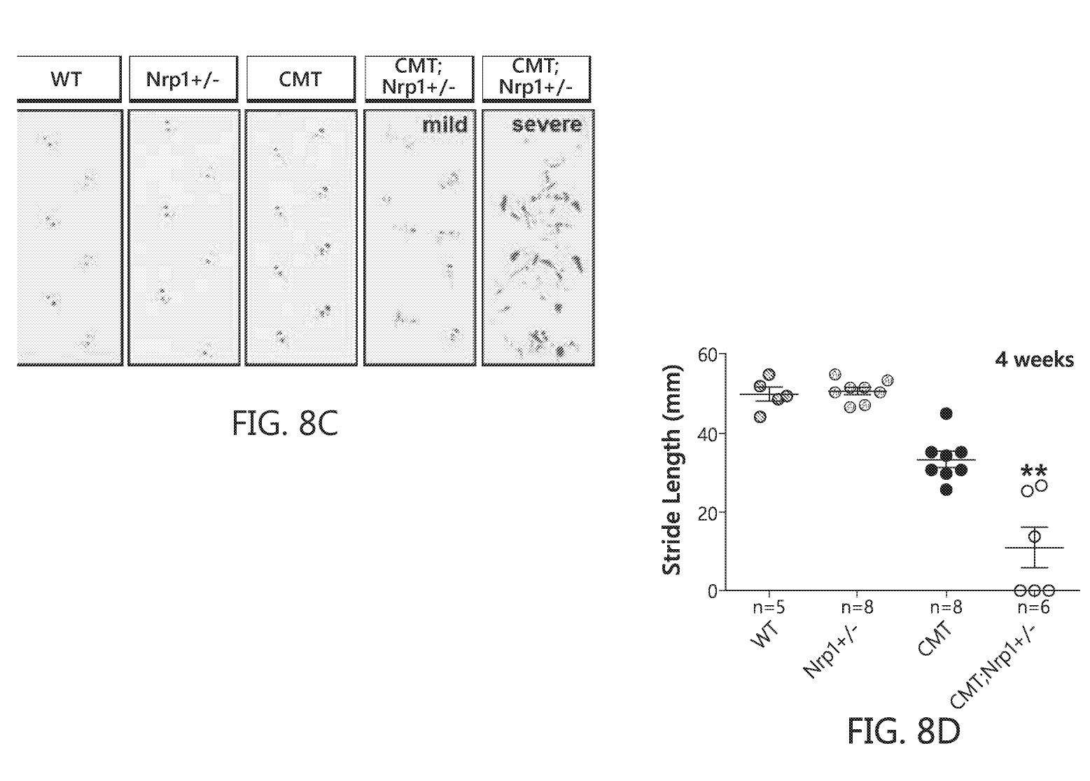

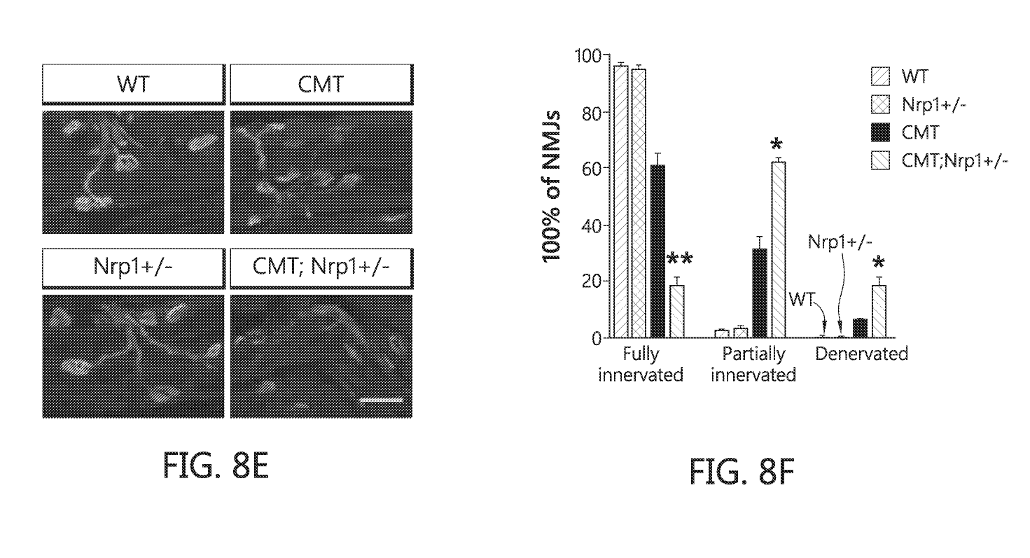

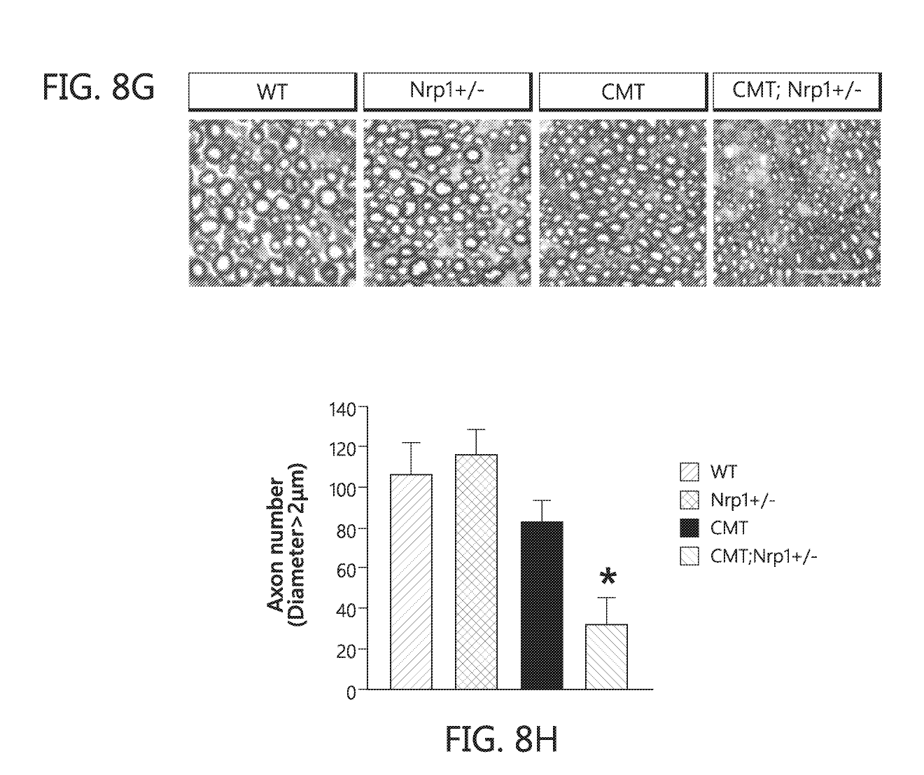

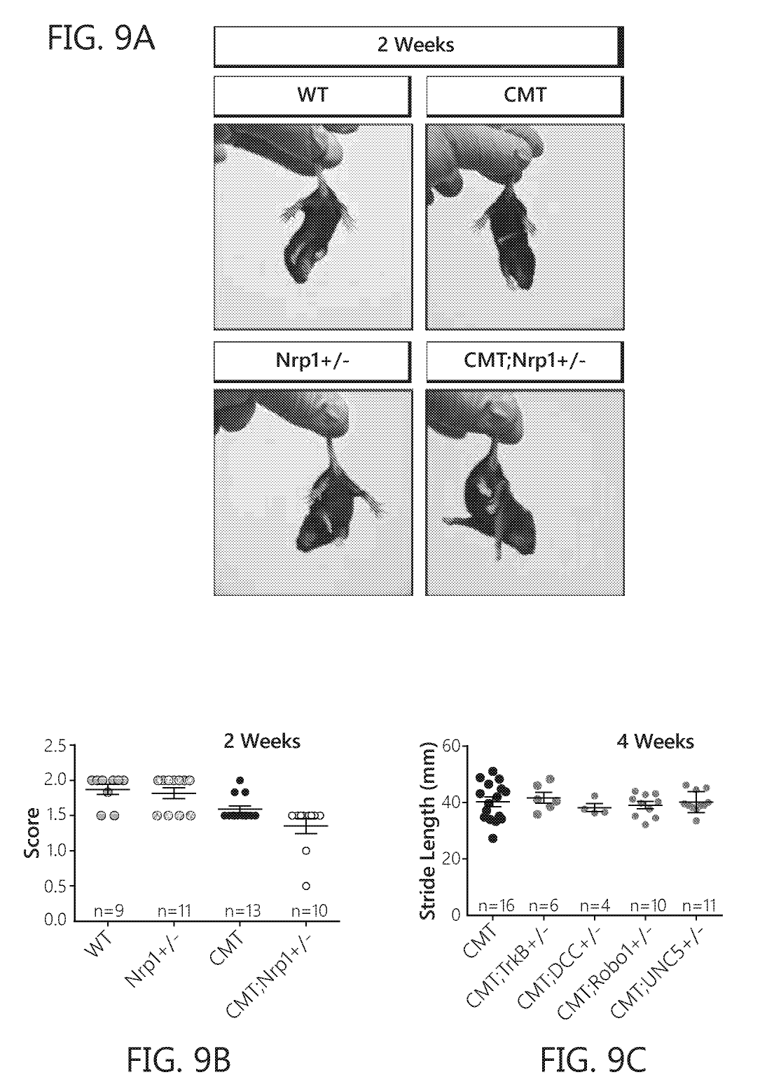

VEGF signaling is thought to protect neurons from a variety of damaging insults (Mackenzie et al., Development 139:1371-1380 (2012)). Intriguingly, deficient VEGF signaling leads to the selective degeneration of motor neurons in mice (Oosthuyse et al., Nat. Genet. 28:131-138 (2001)). To examine whether the VEGF/Nrp1 pathway is involved in motor deficits that arise from GlyRS mutations, the genetic interaction between Gars^{CMT2D} and Nrp1 was determined. Although the data from Example 3 suggest that GlyRS^{CMT2D} attenuates the normal Nrp1-signaling, it is still possible that GlyRS^{CMT2D} might activate aberrant signaling through Nrp1 and cause motor defects. In the first case motor phenotypes should get more severe as Nrp1 gene dosage is reduced in CMT2D-mice, and in the second case motor phenotypes should improve. To test these possibilities, Gars^{CMT2D} mice were intercrossed with Nrp1 heterozygous (Nrp1^{+/-}) animals and characterized the motor behavior of the single and double mutant offspring. At two weeks when motor behavioral changes were not observed in either Gars^{CMT2D} or Nrp1^{+/-} mutant mice, 20% of the compound heterozygous Gars^{CMT2D}/Nrp1 mutant mice had developed neuromuscular dysfunction based on a hindlimb extension test (FIGS. 9A and 9B). At 4 weeks, CMT2D-like symptoms including overt neuromuscular dysfunction and an altered walking stride become apparent in Gars^{CMT2D} mutants, whereas Nrp1^{+/-} mice appeared normal (FIGS. 8A-8D). Strikingly, by four weeks, 50° % of the Gars^{CMT2D}/Nrp1^{+/-} mutant mice had entirely lost the ability to spread their legs and toes (FIGS. 8A and 8B), and exhibited severely abnormal gait patterns (FIGS. 8C and 8D). After postnatal week 4, Gars^{CMT2D}/Nrp1^{+/-} mutant mice began to die. Consistent with the biochemical studies showing GlyRS^{CMT2D} binds poorly to other signaling receptors (see FIG. 3A and FIG. 4A), intercrosses between Gars^{CMT2D} and TrkB^{+/-}, DCC^{+/-}, Robo1^{+/-} and Unc5C^{+/-} heterozygous mice did not worsen the neuromuscular phenotypes in the compound heterozygotes (FIG. 8B and FIG. 9C).

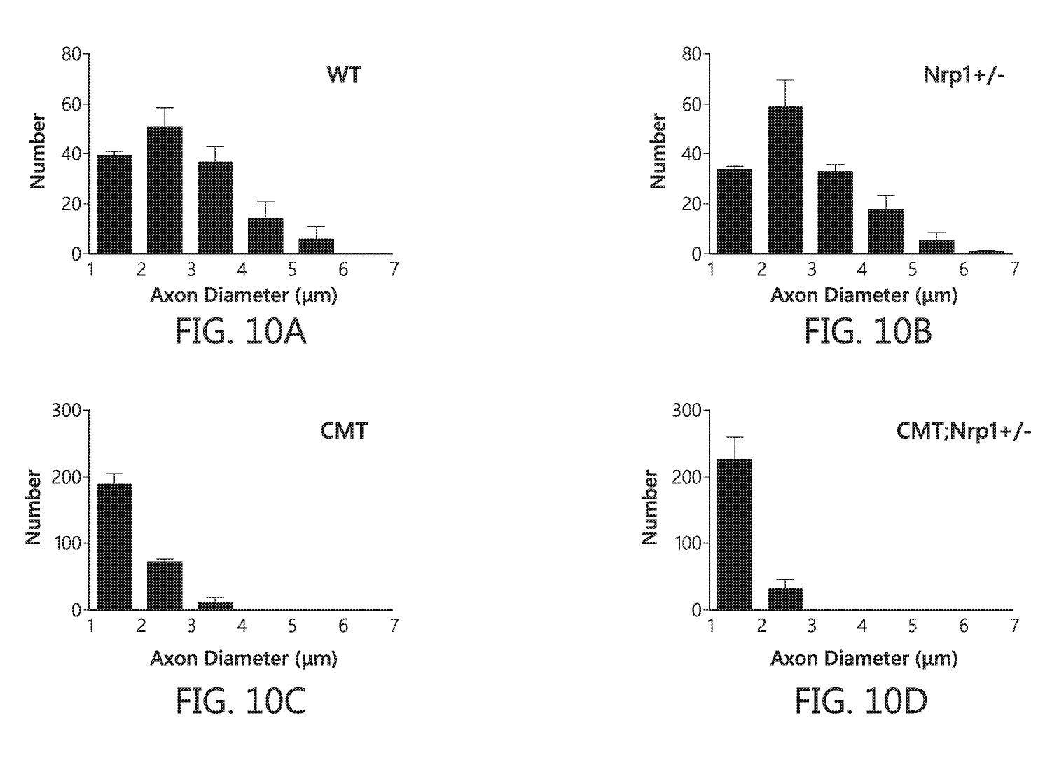

The motor defects in Gars^{CMT2D} and Gars^{CMT2D}/Nrp1^{+/-} mutants were accompanied with significant pathological changes in the peripheral nerves and synaptic contacts with muscle fibers. Neuromuscular junctions (NMJs) displayed a normal apposition of nerve fibers and postsynaptic acetylcholine receptors in wild-type (WT) and Nrp1^{+/-} animals, while partially innervated and completely denervated NMJs were present in four-week Gars^{CMT2D} mutants (FIGS. 8E and 8F). The loss of nerve terminals at NMJs was markedly increased in Gars^{CMT2D}/Nrp1^{+/-} mutants (FIGS. 8E and 8F). Likewise, at four weeks of age many large-diameter axons were absent from the sciatic nerves of Gars^{CMT2D} mutants compared to WT and Nrp1^{+/-} littermates (FIGS. 8G and 8H and FIGS. 10A-10C). The absence of large diameter axons was even more dramatic in four-week Gars^{CMT2D}/Nrp1^{+/-} compound mutants, and was comparable to the extreme axonal dystrophy observed in late-stage CMT2D mutants (FIGS. 8G and 8H and FIG. 10D) (Seburn et al., Neuron 51:715-726 (2006)). These findings demonstrate that Nrp1 is an important genetic modifier of CMT2D pathology and that GlyRS^{CMT2D} antagonizes normal Nrp1-signaling rather than activating aberrant signaling.

### Example 5

### VEGF Treatment Improves Motor Function in CMT2D Mice

This example demonstrates that VEGF treatment significantly ameliorates the loss of motor function in CMT2D mice.

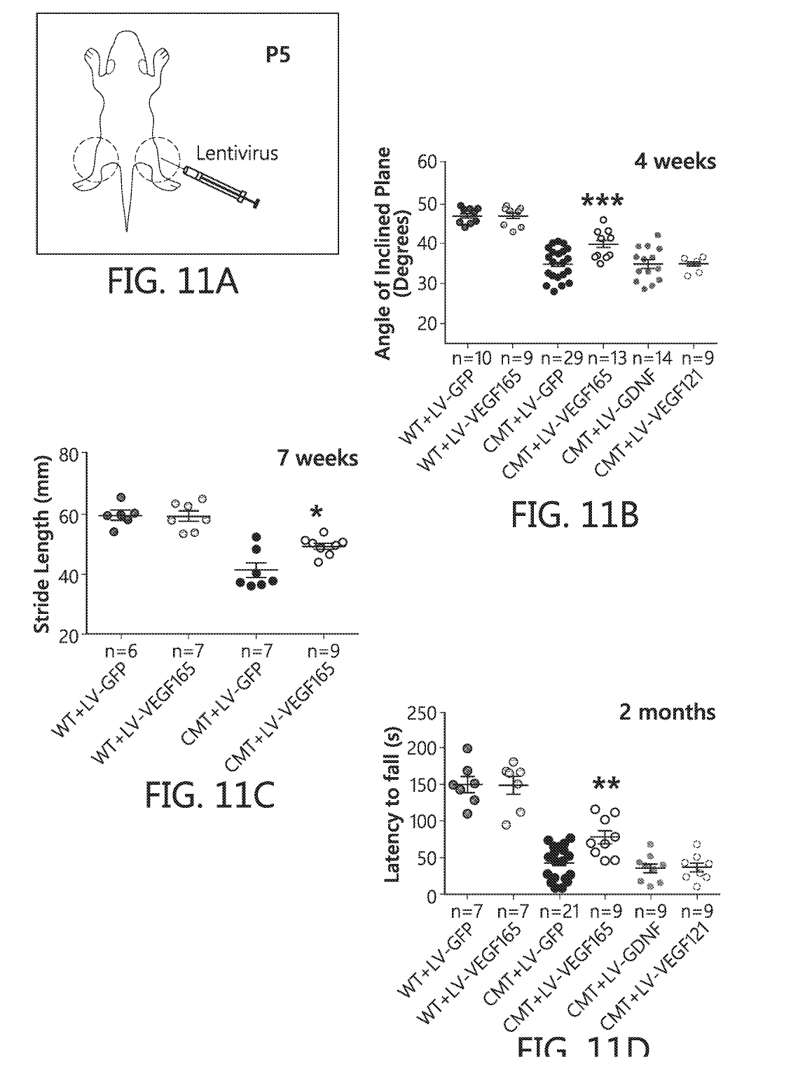

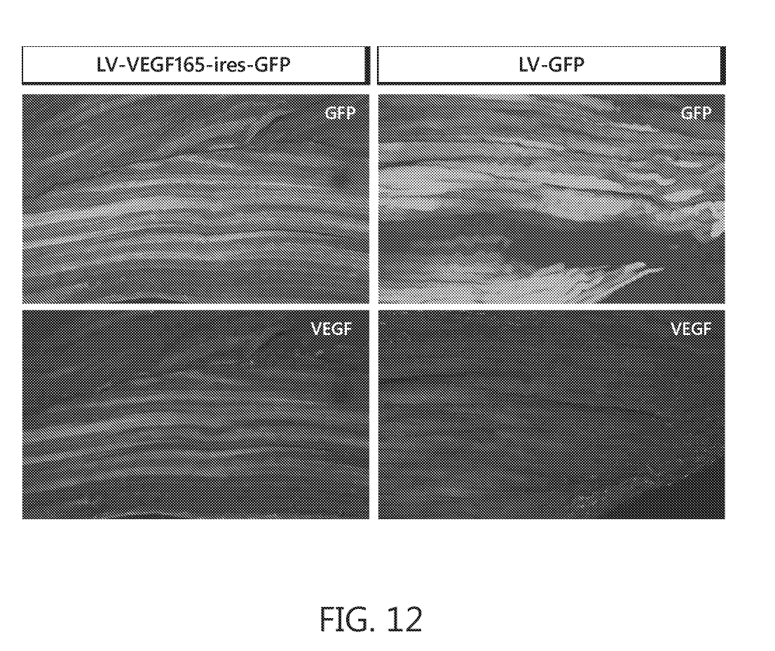

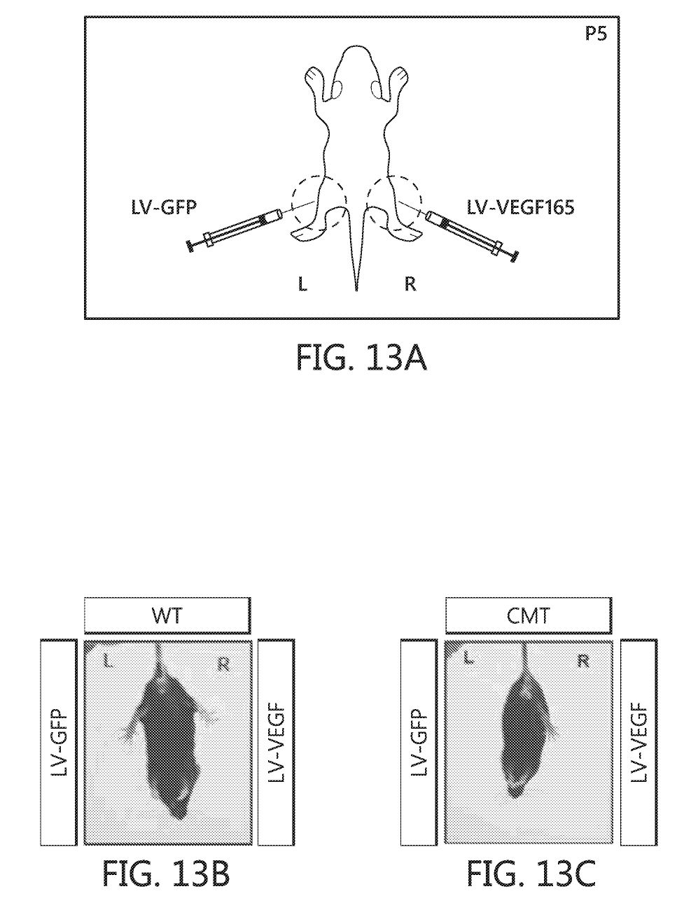

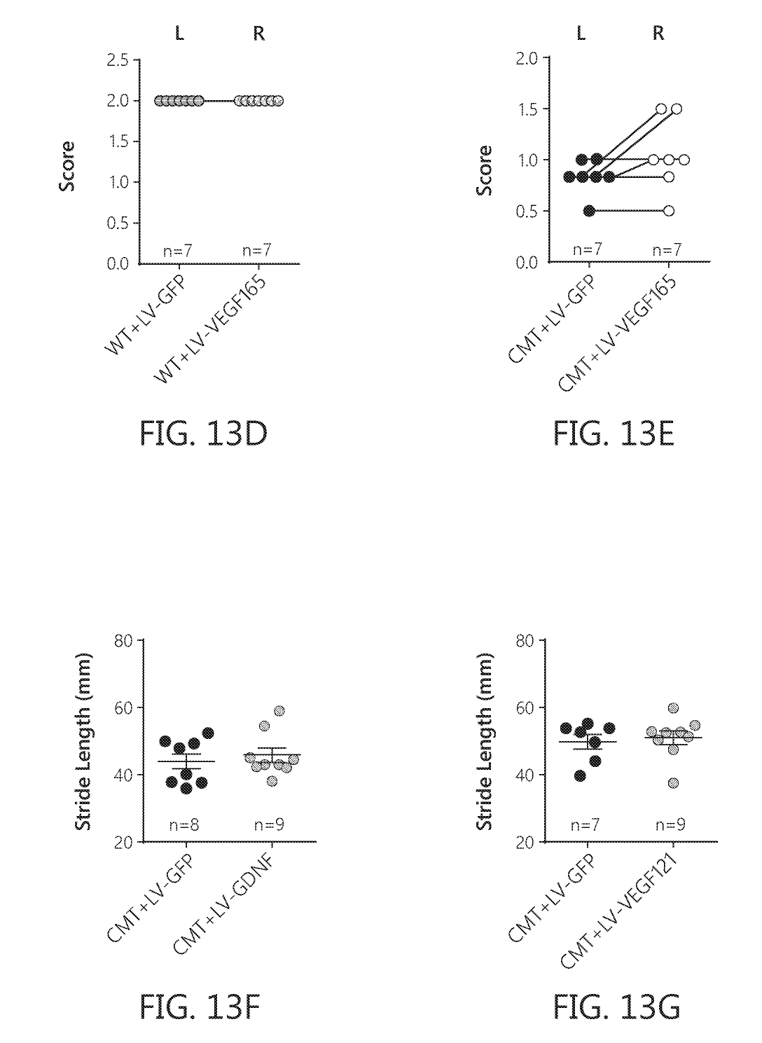

Without being bound by any particular theory, the findings in Example 4, demonstrating that GlyRS^{CMT2D} antagonizes normal Nrp1 signaling prompted the question of whether VEGF overexpression could counteract GlyRS^{CMT2D} and help to slow the loss of motor function in CMT2D mice. A lentiviral vector encoding either VEGF-A165 or GFP was injected bilaterally into the hindlimb muscles of Gars^{CMT2D} mutant mice at postnatal day five prior to the onset of overt motor defects (FIG. 11A and FIG. 12). By four weeks of age, a reduction of limb strength was observed in the control GFP-treated Gars^{CMT2D} animals using an inclined plane test (FIG. 11B). However, VEGF-A165-treated animals retained greater neuromuscular capacity with significantly higher scores (FIG. 11B). By seven weeks, Gars^{CMT2D} animals exhibited a disrupted gait pattern with shortened hindlimb stride length, while VEGF-A165-treated animals maintained a significantly longer walking stride (FIG. 11C). Likewise, VEGF treatment significantly improved the motor performance of Gars^{CMT2D} mutants in the rotarod test (FIG. 11D). To minimize the possible influence from the natural variation in disease progression among individual animals, the effect of VEGF treatment was compared to GFP controls by separately treating each hindlimb from the same animal. Lentiviral vectors encoding VEGF-A165 or GFP were injected unilaterally into each hindlimb of the same Gars^{CMT2D} mutant mouse at postnatal day five (FIG. 13A). At five weeks, GFP-treated hindlimbs developed severe muscle weakness, largely losing their ability to extend. In contrast, the contralateral hindlimbs treated with VEGF-A165 retained significant function (FIGS. 13B-13E). These results suggest that VEGF treatment significantly ameliorates the loss of motor function in CMT2D mice.

A number of neurotropic factors have been tested as broad-spectrum strategies to enhance neuronal survival and treat motor diseases (Wang et al., J. Neurosci. 22:6920-6928 (2002); Acsadi et al., Hum. Gene Ther. 13:1047-1059 (2002); Kaspar et al., Science 301:839-842 (2003); Nayak et al. Biochim Biophys Acta 1762:1128-1138 (2006); Azzouz et al., Nature 429:413-417 (2004)). This raised the possibility that VEGF might slow the progression of CMT2D pathology by functioning as a generic trophic factor rather than a specific agent to restore normal VEGF/Nrp1-signaling. Therefore, the effects of lentivirus-mediated expression of GDNF, a potent neurotrophin that has been used to enhance motor function and survival in mouse models of amyotrophic lateral sclerosis was tested. Unlike VEGF-A165, GDNF failed to slow the disease progression in Gars^{CMT2D} mice (FIGS. 11B and 11D and FIG. 13F). VEGF-mediated motor sparing was tested to determine whether it is dependent upon Nrp1-binding by exploiting the binding specificity of VEGF protein isoforms. VEGF-A121 has overlapping functions with VEGF-A165 but lacks high-affinity Nrp1 binding (Mackenzie et al., Development 139:1371-1380 (2012)). VEGF-A121 treatment failed to ameliorate the loss of motor function in CMT2D 10 animals (FIGS. 11B and 11D and FIG. 13G). These data support a model in which VEGF treatment helps to guard against the motor loss arising from the aberrant activity of GlyRS^{CMT2D} by restoring VEGF/Nrp1 signaling.

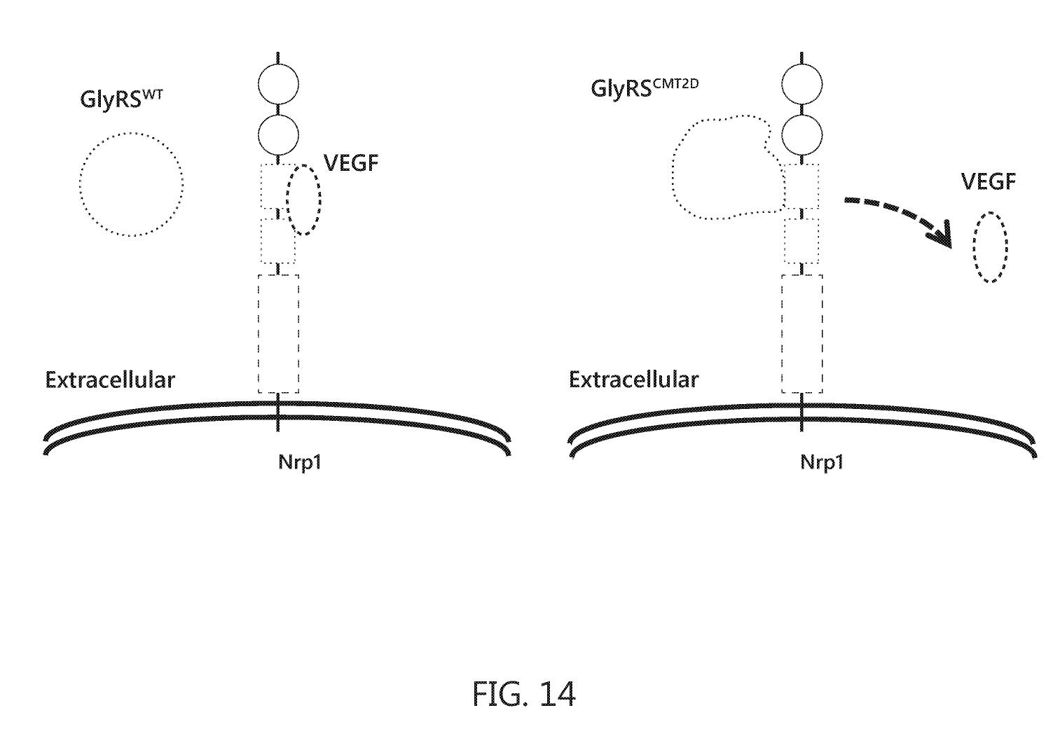

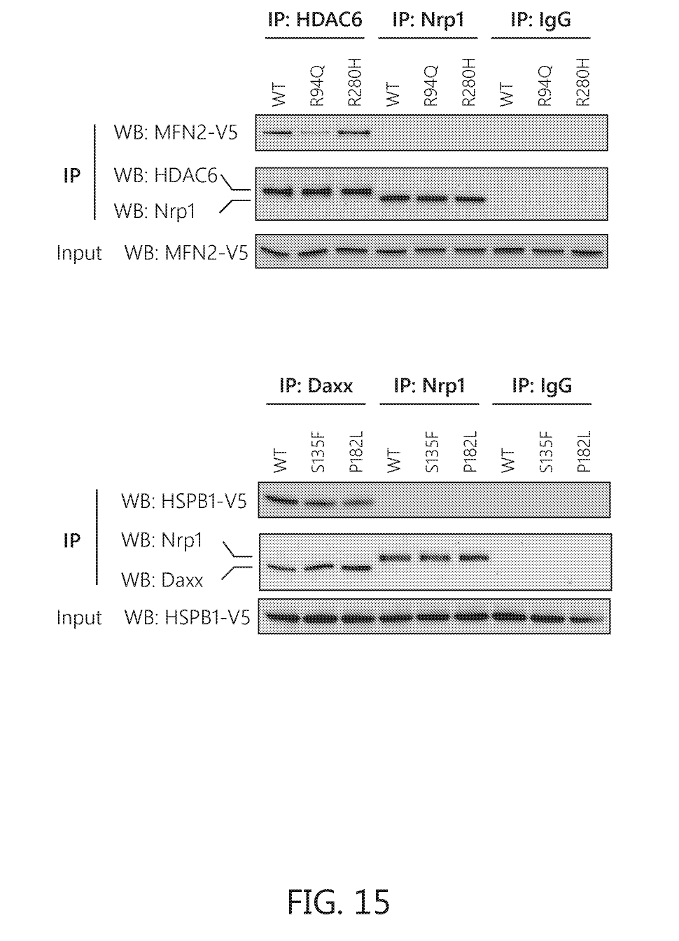

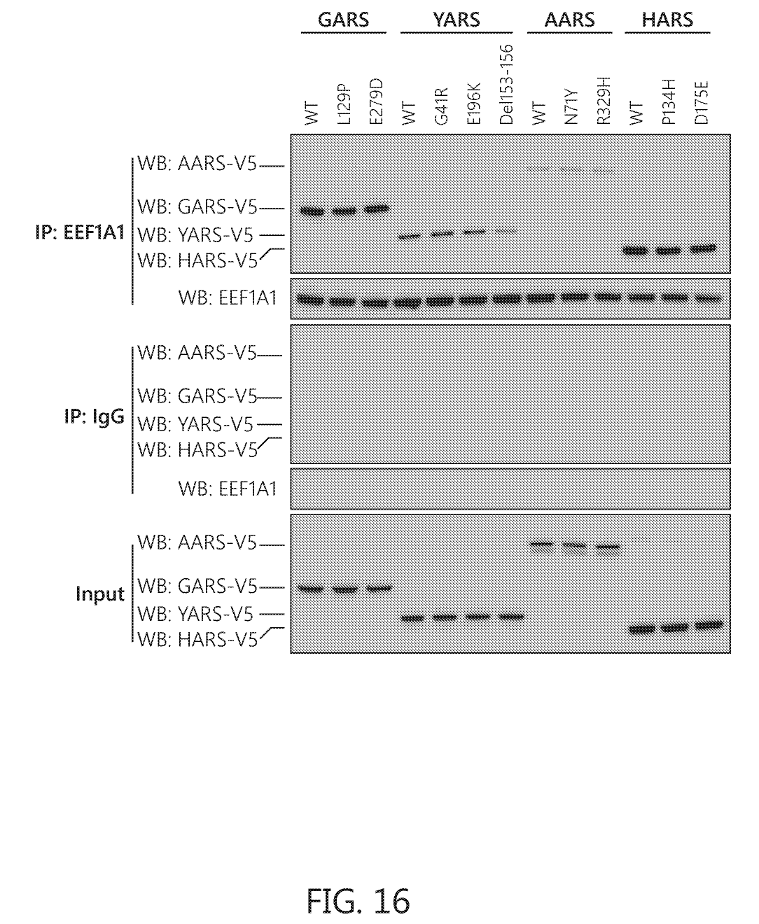

These examples identify the Nrp1 gene as an important genetic modifier for CMT2D, and link the selective pathology of this disease to the neomorphic binding of GlyRS^{CMT2D} to the receptor Nrp1. These findings strongly suggest that the VEGF/Nrp1 pathway is an actionable target for treating CMT2D (FIG. 14). While the exact role of VEGF in the motor system remains poorly defined, VEGF-deficient mice selectively develop symptoms of motor neuron disease over time (Oosthuyse et al., Nat. Genet. 28:131-138 (2001)). The direct antagonism of VEGF/Nrp1 signaling by GlyRS^{CMT2D} found here further indicates that deficient VEGF-signaling may represent a common pathogenic pathway that is susceptible to abnormal activity in other motor neuron diseases. A broad implication from this work is that the molecular basis of selective neuronal vulnerability in neurodegenerative diseases may arise from the neomorphic activity of misfolded proteins interacting with susceptible signaling targets in specific cell types. This conceptual framework may be applied for identifying additional druggable-targets to treat neurodegenerative diseases including other forms of CMT.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1. A method for determining the presence of a mutated aminoacyl tRNA synthetase (aaRS) in a biological sample, comprising:
(a) providing a biological sample that comprises or is suspected of comprising a mutated aaRS;
(b) immobilizing a Neuropilin 1 (Nrp1) protein or a fragment thereof on a solid support;
(c) contacting the biological sample with the immobilized Nrp1 protein under conditions that allows binding of Nrp1 protein to an aaRS to form an immobilized Nrp1-aaRS complex on the solid support;
(d) contacting the solid support with a detectably labeled molecule that specifically binds the aaRS; and
(e) detecting the amount of labeled Nrp1-aaRS complex on the solid support as indicative of the presence or absence of the mutated aaRS in the subject.

2. The method of claim 1, wherein the detectably labeled molecule is an antibody against aaRS or a fragment thereof (anti-aaRS antibody).

3.-5. (canceled)

6. The method of claim 1, wherein step (b) further comprises removing unbound Nrp1 protein or a fragment thereof from the solid support.

7. The method of claim 1, wherein step (c) further comprises washing the solid support to remove any unbound aaRS.

8. The method of claim 1, wherein step (d) further comprises removing unbounded detectably labeled molecule from the solid support.

9. The method of claim 1, further comprising comparing the amount of labeled Nrp1-aaRS complex detected in step (e) with a reference amount of Nrp1-aaRS complex from reference biological samples that do not have a mutated aaRS.

10.-15. (canceled)

16. The method of claim 1, wherein the biological sample comprises neural tissue, neural cells, neuroglia cells, peripheral blood, lymphoblastoid cells, cerebrospinal fluid, ependymal cells, muscle tissue, muscle cells, skin tissues, fibroblasts, or any combination thereof.

17. (canceled)

18. The method of claim 1, wherein the solid support comprises a bead, a microtiter plate, or a combination thereof.

19. The method of claim 1, wherein the Nrp1 protein or the fragment thereof is a recombinant protein.

20. The method of claim 1, wherein the fragment of the Nrp1 protein comprises a b domain of the Nrp1 protein.

21. The method of claim 1, wherein the mutated aaRS is a mutated glycyl-tRNA synthetase (GlyRS), tyrosyl-tRNA synthetase (TyrRS), alanyl-tRNA synthetase (AlaRS), histidyl-tRNA synthetase (HisRS), lysyl-tRNA synthetase (LysRS), or methionyl-tRNA synthetase (MetRS).

22. The method of claim 1, further comprising lysing cells within the biological sample, wherein lysing cells comprises dissociating endogenous Nrp1-aaRS complex in the biological sample.

23. (canceled)

24. The method of claim 1, wherein the biological sample is obtained or derived from a subject suffering from a Charcot-Marie-Tooth (CMT) disease, or a CMT-related neurological disease, or both.

25. (canceled)

26. (canceled)

27. A method for diagnosing a Charcot-Marie-Tooth (CMT) disease or a CMT-related neurological disease in a subject, comprising:
(a) isolating protein complexes comprising Neuropilin 1 (Nrp1) from a biological sample from a subject suspected of having a CMT disease or a CMT-related neurological disease;
(b) determining the amount of vascular endothelial growth factor (VEGF) in the protein complexes isolated in step (a); and
(c) comparing the amount of VEGF determined in step (c) with a reference amount of VEGF in subjects that do not have CMT diseases or CMT-related neurological diseases, whereby lower VEGF amount determined in step (b) indicates that the subject suffers from a CMT disease or a CMT-related neurological disease.

28. The method of claim 27, wherein said determining the amount of VEGF in the protein complexes comprises detecting VEGF using an antibody against VEGF (anti-VEGF antibody), wherein the anti-VEGF antibody is a polyclonal or monoclonal antibody.

29. (canceled)

30. (canceled)

31. The method of claim 27, wherein said determining the amount of VEGF in the protein complexes comprises dissociating the protein complexes.

32. The method of claim 27, further comprising
(d) determining the amount of one or more aminoacyl tRNA synthetases (aaRS) in the protein complexes isolated in step (a).

33. The method of claim 32, wherein at least one of the one or more aaRS is glycyl-tRNA synthetase (GlyRS), tyrosyl-tRNA synthetase (TyrRS), alanyl-tRNA synthetase (AlaRS), histidyl-tRNA synthetase (HisRS), lysyl-tRNA synthetase (LysRS), or methionyl-tRNA synthetase (MetRS).

34. (canceled)

35. The method of claim 32, further comprising comparing the amount of at least one of the one or more aaRS determined in step (e) with a reference amount of the aaRS in subjects that do not have CMT diseases

36.-45. (canceled)

46. The method of claim 27, wherein the biological sample comprises neural tissue, neural cells, neuroglia cells, peripheral blood, lymphoblastoid cells, cerebrospinal fluid, ependymal cells, muscle tissue, muscle cells, skin tissues, fibroblasts, or any combination thereof.

47. (canceled)

48. The method of claim 27, further comprising lysing cells within the biological sample.

49. The method of claim 27, wherein said isolating protein complexes comprising Nrp1 comprises immunoprecipitating the protein complexes using an antibody against Nrp1 (anti-Nrp1 antibody).

50. (canceled)

51. (canceled)

52. The method of claim 49, wherein the anti-Nrp1 antibody binds to a b1 domain or an intracellular domain of Nrp1.

53.-58. (canceled)

59. The method of claim 21, wherein the mutated aaRS is a missense mutant.

60. (canceled)

61. The method of claim 21, wherein the mutated GlyRS comprises at least one amino acid substitution selected from the group consisting of A57V, E71G, P234KY, L129P, D146N, C157R, S211F, L218Q, G240R, P244L, E279D, I280F, H418R, D500N, G526R, S581L, G598A, and a combination thereof, wherein the mutated TyrRS comprises a 4 amino acid deletion of VKOV at positions 153-156, at least one amino acid substitution selected from the group consisting of G41R, D81I, E196K, or a combination thereof, wherein the mutated AlaRS comprises at least one amino acid substitution selected from the group consisting of N71Y, G102R, R329H, E688G, E778A, D893N, and a combination thereof, or wherein the mutated HisRS comprises at least one amino acid substitution selected from the group consisting of T132I, P134H, R1370, D175E, D364Y, and a combination thereof.

62.-90. (canceled)

91. A kit for detecting a mutated aminoacyl tRNA synthetase (aaRS) in a biological sample, comprising:
(a) a cell lysis buffer,
(b) a solid support on which a Neuropilin 1 (Nrp1) protein or a fragment thereof is immobilized; and
(c) a detectably labeled molecule that specifically binds to an aaRS.

92. The kit of claim 91, wherein the Nrp1 protein or a fragment thereof is a recombinant protein.

93. The kit of claim 91, wherein the Nrp1 protein or the fragment thereof comprises a b1 domain of Nrp1 protein.

94. The kit of claim 91, wherein the detectably labeled molecule is an antibody against the aaRS or a fragment thereof (anti-aaRS antibody).

95.-106. (canceled)


SEQUENCE LISTING (54 sequences)

SEQ ID NO: 1 - DNA, 2220 bp, Homo sapiens
atgccctctccgcgtccagtgctgcttagaggtgctcgcgccgctctgctgctgctgctgccgccccggctcttagcccgaccctcgctcctgctccgccggtccctcagcgcggcctcctgccccccgatctccttgcccgccgccgcctcccggagcagcatggacggcgcgggggctgaggaggtgctggcacctctgaggctagcagtgcgccagcagggagatcttgtgcgaaaactcaaagaagataaagcaccccaagtagacgtagacaaagcagtggctgagctcaaagcccgcaagagggttctggaagcaaaggagctggcgttacagcccaaagatgatattgtagaccgagcaaaaatggaagataccctgaagaggaggtttttctatgatcaagcttttgctatttatggaggtgttagtggtctgtatgactttgggccagttggctgtgctttgaagaacaatattattcagacctggaggcagcactttatccaagaggaacagatcctggagatcgattgcaccatgctcacccctgagccagttttaaagacctctggccatgtagacaaatttgctgacttcatggtgaaagacgtaaaaaatggagaatgttttcgtgctgaccatctattaaaagctcatttacagaaattgatgtctgataagaagtgttctgtcgaaaagaaatcagaaatggaaagtgttttggcccagcttgataactatggacagcaagaacttgcggatctttttgtgaactataatgtaaaatctcccattactggaaatgatctatcccctccagtgtcttttaacttaatgttcaagactttcattgggcctggaggaaacatgcctgggtacttgagaccagaaactgcacaggggattttcttgaatttcaaacgacttttggagttcaaccaaggaaagttgccttttgctgctgcccagattggaaattcttttagaaatgagatctcccctcgatctggactgatcagagtcagagaattcacaatggcagaaattgagcactttgtagatcccagtgagaaagaccaccccaagttccagaatgtggcagaccttcacctttatttgtattcagcaaaagcccaggtcagcggacagtccgctcggaaaatgcgcctgggagatgctgttgaacagggtgtgattaataacacagtattaggctatttcattggccgcatctacctctacctcacgaaggttggaatatctccagataaactccgcttccggcagcacatggagaatgagatggcccattatgcctgtgactgttgggatgcagaatccaaaacatcctacggttggattgagattgttggatgtgctgatcgttcctgttatgacctctcctgtcatgcacgagccaccaaagtcccacttgtagctgagaaacctctgaaagaacccaaaacagtcaatgttgttcagtttgaacccagtaagggagcaattggtaaggcatataagaaggatgcaaaactggtgatggagtatcttgccatttgtgatgagtgctacattacagaaatggagatgctgctgaatgagaaaggggaattcacaattgaaactgaagggaaaacatttcagttaacaaaagacatgatcaatgtgaagagattccagaaaacactatatgtggaagaagttgttccgaatgtaattgaaccttccttcggcctgggtaggatcatgtatacggtatttgaacatacattccatgtacgagaaggagatgaacagagaacattcttcagtttccctgctgtagttgctccattcaaatgttccgtcctcccactgagccaaaaccaggagttcatgccatttgtcaaggaattatcggaagccctgaccaggcatggagtatctcacaaagtagacgattcctctgggtcaatcggaaggcgctatgccaggactgatgagattggcgtggcttttggtgtcaccattgactttgacacagtgaacaagaccccccacactgcaactctgagggaccgtgactcaatgcggcagataagagcagagatctctgagctgcccagcatagtccaagacctagccaatggcaacatcacatgggctgatgtggaggccaggtatcctctgtttgaagggcaagagactggtaaaaaagagacaatcgaggaatga

SEQ ID NO: 2 - DNA, 1587 bp, Homo sapiens
atgggggacgctcccagccctgaagagaaactgcaccttatcacccggaacctgcaggaggttctgggggaagagaagctgaaggagatactgaaggagcgggaacttaaaatttactggggaacggcaaccacgggcaaaccacatgtggcttactttgtgcccatgtcaaagattgcagacttcttaaaggcagggtgtgaggtaacaattctgtttgcggacctccacgcatacctggataacatgaaagccccatgggaacttctagaactccgagtcagttactatgagaatgtgatcaaagcaatgctggagagcattggtgtgcccttggagaagctcaagttcatcaaaggcactgattaccagctcagcaaagagtacacactagatgtgtacagactctcctccgtggtcacacagcacgattccaagaaggctggagctgaggtggtaaagcaggtggagcaccctttgctgagtggcctcttataccccggactgcaggctttggatgaagagtatttaaaagtagatgcccaatttggaggcattgatcagagaaagattttcacctttgcagagaagtacctccctgcacttggctattcaaaacgggtccatctgatgaatcctatggttccaggattaacaggcagcaaaatgagctcttcagaagaggagtccaagattgatctccttgatcggaaggaggatgtgaagaaaaaactgaagaaggccttctgtgagccaggaaatgtggagaacaatggggttctgtccttcatcaagcatgtcctttttccccttaagtccgagtttgtgatcctacgagatgagaaatggggtggaaacaaaacctacacagcttacgtggacctggaaaaggactttgctgctgaggttgtacatcctggagacctgaagaattctgttgaagtcgcactgaacaagttgctggatccaatccgggaaaagtttaatacccctgccctgaaaaaactggccagcgctgcctacccagatccctcaaagcagaagccaatggccaaaggccctgccaagaattcagaaccagaggaggtcatcccatcccggctggatatccgtgtggggaaaatcatcactgtggagaagcacccagatgcagacagcctgtatgtagagaagattgacgtgggggaagctgaaccacggactgtggtgagcggcctggtacagttcgtgcccaaggaggaactgcaggacaggctggtagtggtgctgtgcaacctgaaaccccagaagatgagaggagtcgagtcccaaggcatgcttctgtgtgcttctatagaagggataaaccgccaggttgaacctctggaccctccggcaggctctgctcctggtgagcacgtgtttgtgaagggctatgaaaagggccaaccagatgaggagctcaagcccaagaagaaagtcttcgagaagttgcaggctgacttcaaaatttctgaggagtgcatcgcacagtggaagcaaaccaacttcatgaccaagctgggctccatttcctgtaaatcgctgaaaggggggaacattagctag

SEQ ID NO: 3 - DNA, 2907 bp, Homo sapiens
atggactctactctaacagcaagtgaaatccggcagcgatttatagatttcttcaagaggaacgagcatacgtatgttcactcgtctgccaccatcccattggatgaccccactttgctctttgccaatgcaggcatgaaccagtttaaacccattttcctgaacacaattgacccatctcaccccatggcaaagctgagcagagctgccaatacccagaagtgcatccgggctgggggcaaacataatgacctggacgatgtgggcaaggatgtctatcatcacaccttcttcgagatgctgggctcttggtcttttggagattactttaaggaattggcatgtaagatggctctggaactcctcacccaagagtttggcattcccattgaaagactttatgttacttactttggcggggatgaagcagctggcttagaagcagatctggaatgcaaacagatctggcaaaatttggggctggatgacaccaaaatcctcccaggcaacatgaaggataacttctgggagatgggtgacacgggcccctgtggtccttgcagtgagatccactacgaccggattggtggtcgggacgccgcacatcttgtcaaccaggacgaccctaatgtgctggagatctggaaccttgtgttcatccagtataacagggaagctgatggcattctgaaacctcttcccaagaaaagcattgacacagggatgggcctggaacgactggtatctgtgctgcagaataagatgtccaactatgacactgacctttttgtcccttactttgaagccattcagaagggcacaggtgcccgaccatacactgggaaagttggtgctgaggatgccgatgggattgacatggcctaccgggtgctggctgaccacgctcggaccatcactgtggcactggctgatggtggccggcctgacaacacagggcgtggatatgtgttgagacggattctccgccgagctgtccgatacgcccatgaaaagctcaatgccagcaggggcttctttgctacgttagtggatgttgtcgtccagtccctgggagatgcatttcctgagctgaagaaggacccagacatggtgaaggacatcattaatgaagaagaggtgcagtttctcaagactctcagcagagggcgtcgcatcctggacaggaaaattcagagcctgggagacagcaagaccattcccggagacactgcttggctcctctatgacacctatgggtttccagtggatctgactggactgattgctgaagagaagggcctggtggtagacatggatggctttgaagaggagaggaaactggcccagctgaaatcacagggcaagggagctggtggggaagacctcattatgctggacatttacgctatcgaagagctccgggcacggggtctggaggtcacagatgattccccaaagtacaattaccatttggactccagtggtagctatgtatttgagaacacagtggctacggtgatggctctgcgcagggagaagatgttcgtggaagaggtgtccacaggccaggagtgtggagtggtgctggacaagacctgtttctatgctgagcaaggaggccagatctatgacgaaggctacctggtgaaggtggatgacagcagtgaagataaaacagagtttacagtgaagaatgctcaggtccgaggagggtatgtgctacacattggaaccatctacggtgacctgaaagtgggggatcaggtctggctgtttattgatgagccccgacgaagacccatcatgagcaaccacacagctacgcacattctgaacttcgccctgcgctcagtgcttggggaagctgaccagaaaggctcattggttgctcctgaccgcctcagatttgactttactgccaagggagccatgtccacccaacagatcaagaaggctgaagagattgctaatgagatgattgaggcagccaaggccgtctatacccaggattgccccctggcagcagcgaaagccatccagggcctacgggctgtgtttgatgagacctatcctgaccctgtgcgagtcgtctccattggggtcccggtgtccgagttgctggatgacccctctgggcctgctggctccctgacttctgttgagttctgtgggggaacgcacctgcggaactcgagtcatgcaggagcttttgtgatcgtgacggaagaagccattgccaagggtatccggaggattgtggctgtcacaggtgccgaggcccagaaggccctcaggaaagcagagagcttgaagaaatgtctctctgtcatggaagccaaagtgaaggctcagactgctccaaacaaggatgtgcagagggagatcgctgaccttggagaggccctggccactgcagtcatcccccagtggcagaaggatgaattgcgggagactctcaaatccctaaagaaggtcatggatgacttggaccgagccagcaaagccgatgtccagaaacgagtgttagagaagacgaagcagttcatcgacagcaaccccaaccagcctcttgtcatcctggagatggagagcggcgcctcagccaaggccctgaatgaagccttgaagctcttcaagatgcactcccctcagacttctgccatgctcttcacggtggacaatgaggctggcaagatcacgtgcctgtgtcaagttccccagaatgcagccaatcggggcttaaaagccagcgagtgggtgcagcaggtgtcaggcttgatggacggtaaaggtggtggcaaggatgtgtctgcacaggccacaggcaagaacgttggctgcctgcaggaggcgctgcagctggccacttccttcgcccagctgcgcctcggggatgtaaagaactga

SEQ ID NO: 4 - DNA, 1410 bp, Homo sapiens
atggcagagcgtgcggcgctggaggagctggtgaaacttcagggagagcgcgtgcgaggcctcaagcagcagaaggccagcgccgagctgatcgaggaggaggtggcgaaactcctgaaactgaaggcacagctgggtcctgatgaaagcaaacagaaatttgtgctcaaaacccccaaggaaacactgatgggaaagtatggggaagactccaagcttatctatgacctgaaggaccagggcggggagctcctgtcccttcgctatgacctcactgttccttttgctcggtatttggcaatgaataaactgaccaacattaaacgctaccacatagcaaaggtatatcggcgggataacccagccatgacccgtggccgataccgggaattctaccagtgtgattttgacattgctgggaactttgatcccatgatccctgatgcagagtgcctgaagatcatgtgcgagatcctgagttcacttcagataggcgacttcctggtcaaggtaaacgatcgacgcattctagatgggatgtttgctatctgtggtgtttctgacagcaagttccgtaccatctgctcctcagtagacaagctggacaaggtgtcctgggaagaggtgaagaatgagatggtgggagagaagggccttgcacctgaggtggctgaccgcattggggactatgtccagcaacatggtggggtatccctggtggaacagctgctccaggatcctaaactatcccaaaacaagcaggccttggagggcctgggagacctgaagttgctctttgagtacctgaccctatttggcattgatgacaaaatctcctttgacctgagccttgctcgagggctggattactacactggggtgatctatgaggcagtgctgctacagaccccagcccaggcaggggaagagcccctgggtgtgggcagtgtggctgctggaggacgctatgatgggctagtgggcatgttcgaccccaaagggcgcaaggtgccatgtgtggggctcagcattggggtggagcggattttctccatcgtggaacagagactagaggctttggaggagaagatacggaccacggagacacaggtgcttgtggcatctgcacagaagaagctgctagaggaaagactaaagcttgtctcagaactgtgggatgctgggatcaaggctgagctgctgtacaagaagaacccaaagctactgaaccagttacagtactgtgaggaggcaggcatcccactggtggctatcatcggcgagcaggaactcaaggatggggtcatcaagctccgttcagtgacgagcagggaagaggtggatgtccgaagagaagaccttgtggaggaaatcaaaaggagaacaggccagcccctctgcatctgctga

SEQ ID NO: 5 - DNA, 1878 bp, Homo sapiens
atgttgacgcaagctgctgtaaggcttgttagggggtccctgcgcaaaacctcctgggcagagtggggtcacagggaactgcgactgggtcaacttgctcctttcacagcgcctcacaaggacaagtcattttctgatcaaagaagtgagctgaagagacgcctgaaagctgagaagaaagtagcagagaaggaggccaaacagaaagagctcagtgagaaacagctaagccaagccactgctgctgccaccaaccacaccactgataatggtgtgggtcctgaggaagagagcgtggacccaaatcaatactacaaaatccgcagtcaagcaattcatcagctgaaggtcaatggggaagacccatacccacacaagttccatgtagacatctcactcactgacttcatccaaaaatatagtcacctgcagcctggggatcacctgactgacatcaccttaaaggtggcaggtaggatccatgccaaaagagcttctgggggaaagctcatcttctatgatcttcgaggagagggggtgaagttgcaagtcatggccaattccagaaattataaatcagaagaagaatttattcatattaataacaaactgcgtcggggagacataattggagttcaggggaatcctggtaaaaccaagaagggtgagctgagcatcattccgtatgagatcacactgctgtctccctgtttgcatatgttacctcatcttcactttggcctcaaagacaaggaaacaaggtatcgccagagatacttggacttgatcctgaatgactttgtgaggcagaaatttatcatccgctctaagatcatcacatatataagaagtttcttagatgagctgggattcctagagattgaaactcccatgatgaacatcatcccagggggagccgtggccaagcctttcatcacttatcacaacgagctggacatgaacttatatatgagaattgctccagaactctatcataagatgcttgtggttggtggcatcgaccgggtttatgaaattggacgccagttccggaatgaggggattgatttgacgcacaatcctgagttcaccacctgtgagttctacatggcctatgcagactatcacgatctcatggaaatcacggagaagatggtttcagggatggtgaagcatattacaggcagttacaaggtcacctaccacccagatggcccagagggccaagcctacgatgttgacttcaccccacccttccggcgaatcaacatggtagaagagcttgagaaagccctggggatgaagctgccagaaacgaacctctttgaaactgaagaaactcgcaaaattcttgatgatatctgtgtggcaaaagctgttgaatgccctccacctcggaccacagccaggctccttgacaagcttgttggggagttcctggaagtgacttgcatcaatcctacattcatctgtgatcacccacagataatgagccctttggctaaatggcaccgctctaaagagggtctgactgagcgctttgagctgtttgtcatgaagaaagagatatgcaatgcgtatactgagctgaatgatcccatgcggcagcggcagctttttgaagaacaggccaaggccaaggctgcaggtgatgatgaggccatgttcatagatgaaaacttctgtactgccctggaatatgggctgccccccacagctggctggggcatgggcattgatcgagtcgccatgtttctcacggactccaacaacatcaaggaagtacttctgtttcctgccatgaaacccgaagacaagaaggagaatgtagcaaccactgatacactggaaagcacaacagttggcacttctgtctag

SEQ ID NO: 6 - DNA, 2703 bp, Homo sapiens
atgagactgttcgtgagtgatggcgtcccgggttgcttgccggtgctggccgccgccgggagagcccggggcagagcagaggtgctcatcagcactgtaggcccggaagattgtgtggtcccgttcctgacccggcctaaggtccctgtcttgcagctggatagcggcaactacctcttctccactagtgcaatctgccgatatttttttttgttatctggctgggagcaagatgacctcactaaccagtggctggaatgggaagcgacagagctgcagccagctttgtctgctgccctgtactatttagtggtccaaggcaagaagggggaagatgttcttggttcagtgcggagagccctgactcacattgaccacagcttgagtcgtcagaactgtcctttcctggctggggagacagaatctctagccgacattgttttgtggggagccctatacccattactgcaagatcccgcctacctccctgaggagctgagtgccctgcacagctggttccagacactgagtacccaggaaccatgtcagcgagctgcagagactgtactgaaacagcaaggtgtcctggctctccggccttacctccaaaagcagccccagcccagccccgctgagggaagggctgtcaccaatgagcctgaggaggaggagctggctaccctatctgaggaggagattgctatggctgttactgcttgggagaagggcctagaaagtttgcccccgctgcggccccagcagaatccagtgttgcctgtggctggagaaaggaatgtgctcatcaccagtgccctcccttacgtcaacaatgtcccccaccttgggaacatcattggttgtgtgctcagtgccgatgtctttgccaggtactctcgcctccgccagtggaacaccctctatctgtgtgggacagatgagtatggtacagcaacagagaccaaggctctggaggagggactaaccccccaggagatctgcgacaagtaccacatcatccatgctgacatctaccgctggtttaacatttcgtttgatatttttggtcgcaccaccactccacagcagaccaaaatcacccaggacattttccagcagttgctgaaacgaggttttgtgctgcaagatactgtggagcaactgcgatgtgagcactgtgctcgcttcctggctgaccgcttcgtggagggcgtgtgtcccttctgtggctatgaggaggctcggggtgaccagtgtgacaagtgtggcaagctcatcaatgctgtcgagcttaagaagcctcagtgtaaagtctgccgatcatgccctgtggtgcagtcgagccagcacctgtttctggacctgcctaagctggagaagcgactggaggagtggttggggaggacattgcctggcagtgactggacacccaatgcccagtttatcacccgttcttggcttcgggatggcctcaagccacgctgcataacccgagacctcaaatggggaacccctgtacccttagaaggttttgaagacaaggtattctatgtctggtttgatgccactattggctatctgtccatcacagccaactacacagaccagtgggagagatggtggaagaacccagagcaagtggacctgtatcagttcatggccaaagacaatgttcctttccatagcttagtctttccttgctcagccctaggagctgaggataactataccttggtcagccacctcattgctacagagtacctgaactatgaggatgggaaattctctaagagccgcggtgtgggagtgtttggggacatggcccaggacacggggatccctgctgacatctggcgcttctatctgctgtacattcggcctgagggccaggacagtgctttctcctggacggacctgctgctgaagaataattctgagctgcttaacaacctgggcaacttcatcaacagagctgggatgtttgtgtctaagttctttgggggctatgtgcctgagatggtgctcacccctgatgatcagcgcctgctggcccatgtcaccctggagctccagcactatcaccagctacttgagaaggttcggatccgggatgccttgcgcagtatcctcaccatatctcgacatggcaaccaatatattcaggtgaatgagccctggaagcggattaaaggcagtgaggctgacaggcaacgggcaggaacagtgactggcttggcagtgaatatagctgccttgctctctgtcatgcttcagccttacatgcccacggttagtgccacaatccaggcccagctgcagctcccacctccagcctgcagtatcctgctgacaaacttcctgtgtaccttaccagcaggacaccagattggcacagtcagtcccttgttccaaaaattggaaaatgaccagattgaaagtttaaggcagcgctttggagggggccaggcaaaaacgtccccgaagccagcagttgtagagactgttacaacagccaagccacagcagatacaagcgctgatggatgaagtgacaaaacaaggaaacattgtccgagaactgaaagcacaaaaggcagacaagaacgaggttgctgcggaggtggcgaaactcttggatctaaagaaacagttggctgtagctgaggggaaaccccctgaagcccctaaaggcaagaagaaaaagtaa

SEQ ID NO: 7 - DNA, 1935 bp, Homo sapiens
atggagagggggctgccgctcctctgcgccgtgctcgccctcgtcctcgccccggccggcgcttttcgcaacgataaatgtggcgatactataaaaattgaaagccccgggtaccttacatctcctggttatcctcattcttatcacccaagtgaaaaatgcgaatggctgattcaggctccggacccataccagagaattatgatcaacttcaaccctcacttcgatttggaggacagagactgcaagtatgactacgtggaagtcttcgatggagaaaatgaaaatggacattttaggggaaagttctgtggaaagatagcccctcctcctgttgtgtcttcagggccatttctttttatcaaatttgtctctgactacgaaacacatggtgcaggattttccatacgttatgaaattttcaagagaggtcctgaatgttcccagaactacacaacacctagtggagtgataaagtcccccggattccctgaaaaatatcccaacagccttgaatgcacttatattgtctttgcgccaaagatgtcagagattatcctggaatttgaaagctttgacctggagcctgactcaaatcctccaggggggatgttctgtcgctacgaccggctagaaatctgggatggattccctgatgttggccctcacattgggcgttactgtggacagaaaacaccaggtcgaatccgatcctcatcgggcattctctccatggttttttacaccgacagcgcgatagcaaaagaaggtttctcagcaaactacagtgtcttgcagagcagtgtctcagaagatttcaaatgtatggaagctctgggcatggaatcaggagaaattcattctgaccagatcacagcttcttcccagtatagcaccaactggtctgcagagcgctcccgcctgaactaccctgagaatgggtggactcccggagaggattcctaccgagagtggatacaggtagacttgggccttctgcgctttgtcacggctgtcgggacacagggcgccatttcaaaagaaaccaagaagaaatattatgtcaagacttacaagatcgacgttagctccaacggggaagactggatcaccataaaagaaggaaacaaacctgttctctttcagggaaacaccaaccccacagatgttgtggttgcagtattccccaaaccactgataactcgatttgtccgaatcaagcctgcaacttgggaaactggcatatctatgagatttgaagtatacggttgcaagataacagattatccttgctctggaatgttgggtatggtgtctggacttatttctgactcccagatcacatcatccaaccaaggggacagaaactggatgcctgaaaacatccgcctggtaaccagtcgctctggctgggcacttccacccgcacctcattcctacatcaatgagtggctccaaatagacctgggggaggagaagatcgtgaggggcatcatcattcagggtgggaagcaccgagagaacaaggtgttcatgaggaagttcaagatcgggtacagcaacaacggctcggactggaagatgatcatggatgacagcaaacgcaaggcgaagtcttttgagggcaacaacaactatgatacacctgagctgcggacttttccagctctctccacgcgattcatcaggatctaccccgagagagccactcatggcggactggggctcagaatggagctgctgggctgtgaagtggaagcccctacagctggaccgaccactcccaacgggaacttggtggatgaatgtgatgacgaccaggccaactgccacagtggaacaggtgatgacttccagctcacaggtggcaccactgtgctggccacagaaaagcccacggtcatagacagcaccatacaatcaggtatcaaataa

SEQ ID NO: 8 - DNA, 1239 bp, Homo sapiens
ctgacggacagacagacagacaccgcccccagccccagctaccacctcctccccggccggcggcggacagtggacgcggcggcgagccgcgggcaggggccggagcccgcgcccggaggcggggtggagggggtcggggctcgcggcgtcgcactgaaacttttcgtccaacttctgggctgttctcgcttcggaggagccgtggtccgcgcgggggaagccgagccgagcggagccgcgagaagtgctagctcgggccgggaggagccgcagccggaggagggggaggaggaagaagagaaggaagaggagagggggccgcagtggcgactcggcgctcggaagccgggctcatggacgggtgaggcggcggtgtgcgcagacagtgctccagccgcgcgcgctccccaggccctggcccgggcctcgggccggggaggaagagtagctcgccgaggcgccgaggagagcgggccgccccacagcccgagccggagagggagcgcgagccgcgccggccccggtcgggcctccgaaaccatgaactttctgctgtcttgggtgcattggagccttgccttgctgctctacctccaccatgccaagtggtcccaggctgcacccatggcagaaggaggagggcagaatcatcacgaagtggtgaagttcatggatgtctatcagcgcagctactgccatccaatcgagaccctggtggacatcttccaggagtaccctgatgagatcgagtacatcttcaagccatcctgtgtgcccctgatgcgatgcgggggctgctgcaatgacgagggcctggagtgtgtgcccactgaggagtccaacatcaccatgcagattatgcggatcaaacctcaccaaggccagcacataggagagatgagcttcctacagcacaacaaatgtgaatgcagaccaaagaaagatagagcaagacaagaaaaaaaatcagttcgaggaaagggaaaggggcaaaaacgaaagcgcaagaaatcccggtataagtcctggagcgtgtacgttggtgcccgctgctgtctaatgccctggagcctccctggcccccatccctgtgggccttgctcagagcggagaaagcatttgtttgtacaagatccgcagacgtgtaaatgttcctgcaaaaacacagactcgcgttgcaaggcgaggcagcttgagttaaacgaacgtacttgcagatgtgacaagccgaggcggtga

SEQ ID NO: 9 - DNA, 468 bp, Homo sapiens
ttcaaatgtatggaagctctgggcatggaatcaggagaaattcattctgaccagatcacagcttcttcccagtatagcaccaactggtctgcagagcgctcccgcctgaactaccctgagaatgggtggactcccggagaggattcctaccgagagtggatacaggtagacttgggccttctgcgctttgtcacggctgtcgggacacagggcgccatttcaaaagaaaccaagaagaaatattatgtcaagacttacaagatcgacgttagctccaacggggaagactggatcaccataaaagaaggaaacaaacctgttctctttcagggaaacaccaaccccacagatgttgtggttgcagtattccccaaaccactgataactcgatttgtccgaatcaagcctgcaacttgggaaactggcatatctatgagatttgaagtatacggttgcaagataacagat

SEQ ID NO: 10 - protein, 739 aa, Homo sapiens
MPSPRPVLLRGARAALLLLLPPRLLARPSLLLRRSLSAASCPPISLPAAASRSSMDGAGAEEVLAPLRLAVRQQGDLVRKLKEDKAPQVDVDKAVAELKARKRVLEAKELALQPKDDIVDRAKMEDTLKRRFFYDQAFAIYGGVSGLYDFGPVGCALKNNIIQTWRQHFIQEEQILEIDCTMLTPEPVLKTSGHVDKFADFMVKDVKNGECFRADHLLKAHLQKLMSDKKCSVEKKSEMESVLAQLDNYGQQELADLFVNYNVKSPITGNDLSPPVSFNLMFKTFIGPGGNMPGYLRPETAQGIFLNFKRLLEFNQGKLPFAAAQIGNSFRNEISPRSGLIRVREFTMAEIEHFVDPSEKDHPKFQNVADLHLYLYSAKAQVSGQSARKMRLGDAVEQGVINNTVLGYFIGRIYLYLTKVGISPDKLRFRQHMENEMAHYACDCWDAESKTSYGWIEIVGCADRSCYDLSCHARATKVPLVAEKPLKEPKTVNVVQFEPSKGAIGKAYKKDAKLVMEYLAICDECYITEMEMLLNEKGEFTIETEGKTFQLTKDMINVKRFQKTLYVEEVVPNVIEPSFGLGRIMYTVFEHTFHVREGDEQRTFFSFPAVVAPFKCSVLPLSQNQEFMPFVKELSEALTRHGVSHKVDDSSGSIGRRYARTDEIGVAFGVTIDFDTVNKTPHTATLRDRDSMRQIRAEISELPSIVQDLANGNITWADVEARYPLFEGQETGKKETIEE

SEQ ID NO: 11 - protein, 739 aa, Artificial Sequence
  GARS A57V
MPSPRPVLLRGARAALLLLLPPRLLARPSLLLRRSLSAASCPPISLPAAASRSSMDGAGAEEVLAPLRLAVRQQGDLVRKLKEDKAPQVDVDKAVAELKARKRVLEAKELVLQPKDDIVDRAKMEDTLKRRFFYDQAFAIYGGVSGLYDFGPVGCALKNNIIQTWRQHFIQEEQILEIDCTMLTPEPVLKTSGHVDKFADFMVKDVKNGECFRADHLLKAHLQKLMSDKKCSVEKKSEMESVLAQLDNYGQQELADLFVNYNVKSPITGNDLSPPVSFNLMFKTFIGPGGNMPGYLRPETAQGIFLNFKRLLEFNQGKLPFAAAQIGNSFRNEISPRSGLIRVREFTMAEIEHFVDPSEKDHPKFQNVADLHLYLYSAKAQVSGQSARKMRLGDAVEQGVINNTVLGYFIGRIYLYLTKVGISPDKLRFRQHMENEMAHYACDCWDAESKTSYGWIEIVGCADRSCYDLSCHARATKVPLVAEKPLKEPKTVNVVQFEPSKGAIGKAYKKDAKLVMEYLAICDECYITEMEMLLNEKGEFTIETEGKTFQLTKDMINVKRFQKTLYVEEVVPNVIEPSFGLGRIMYTVFEHTFHVREGDEQRTFFSFPAVVAPFKCSVLPLSQNQEFMPFVKELSEALTRHGVSHKVDDSSGSIGRRYARTDEIGVAFGVTIDFDTVNKTPHTATLRDRDSMRQIRAEISELPSIVQDLANGNITWADVEARYPLFEGQETGKKETIEE

SEQ ID NO: 12 - protein, 739 aa, Artificial Sequence
  GARS E71G
MPSPRPVLLRGARAALLLLLPPRLLARPSLLLRRSLSAASCPPISLPAAASRSSMDGAGAEEVLAPLRLAVRQQGDLVRKLKEDKAPQVDVDKAVAELKARKRVLEAKELALQPKDDIVDRAKMGDTLKRRFFYDQAFAIYGGVSGLYDFGPVGCALKNNIIQTWRQHFIQEEQILEIDCTMLTPEPVLKTSGHVDKFADFMVKDVKNGECFRADHLLKAHLQKLMSDKKCSVEKKSEMESVLAQLDNYGQQELADLFVNYNVKSPITGNDLSPPVSFNLMFKTFIGPGGNMPGYLRPETAQGIFLNFKRLLEFNQGKLPFAAAQIGNSFRNEISPRSGLIRVREFTMAEIEHFVDPSEKDHPKFQNVADLHLYLYSAKAQVSGQSARKMRLGDAVEQGVINNTVLGYFIGRIYLYLTKVGISPDKLRFRQHMENEMAHYACDCWDAESKTSYGWIEIVGCADRSCYDLSCHARATKVPLVAEKPLKEPKTVNVVQFEPSKGAIGKAYKKDAKLVMEYLAICDECYITEMEMLLNEKGEFTIETEGKTFQLTKDMINVKRFQKTLYVEEVVPNVIEPSFGLGRIMYTVFEHTFHVREGDEQRTFFSFPAVVAPFKCSVLPLSQNQEFMPFVKELSEALTRHGVSHKVDDSSGSIGRRYARTDEIGVAFGVTIDFDTVNKTPHTATLRDRDSMRQIRAEISELPSIVQDLANGNITWADVEARYPLFEGQETGKKETIEE

SEQ ID NO: 13 - protein, 739 aa, Artificial Sequence
  GARS L129P
MPSPRPVLLRGARAALLLLLPPRLLARPSLLLRRSLSAASCPPISLPAAASRSSMDGAGAEEVLAPLRLAVRQQGDLVRKLKEDKAPQVDVDKAVAELKARKRVLEAKELALQPKDDIVDRAKMEDTLKRRFFYDQAFAIYGGVSGLYDFGPVGCALKNNIIQTWRQHFIQEEQILEIDCTMPTPEPVLKTSGHVDKFADFMVKDVKNGECFRADHLLKAHLQKLMSDKKCSVEKKSEMESVLAQLDNYGQQELADLFVNYNVKSPITGNDLSPPVSFNLMFKTFIGPGGNMPGYLRPETAQGIFLNFKRLLEFNQGKLPFAAAQIGNSFRNEISPRSGLIRVREFTMAEIEHFVDPSEKDHPKFQNVADLHLYLYSAKAQVSGQSARKMRLGDAVEQGVINNTVLGYFIGRIYLYLTKVGISPDKLRFRQHMENEMAHYACDCWDAESKTSYGWIEIVGCADRSCYDLSCHARATKVPLVAEKPLKEPKTVNVVQFEPSKGAIGKAYKKDAKLVMEYLAICDECYITEMEMLLNEKGEFTIETEGKTFQLTKDMINVKRFQKTLYVEEVVPNVIEPSFGLGRIMYTVFEHTFHVREGDEQRTFFSFPAVVAPFKCSVLPLSQNQEFMPFVKELSEALTRHGVSHKVDDSSGSIGRRYARTDEIGVAFGVTIDFDTVNKTPHTATLRDRDSMRQIRAEISELPSIVQDLANGNITWADVEARYPLFEGQETGKKETIEE

SEQ ID NO: 14 - protein, 739 aa, Artificial Sequence
  GARS D146N
MPSPRPVLLRGARAALLLLLPPRLLARPSLLLRRSLSAASCPPISLPAAASRSSMDGAGAEEVLAPLRLAVRQQGDLVRKLKEDKAPQVDVDKAVAELKARKRVLEAKELALQPKDDIVDRAKMEDTLKRRFFYDQAFAIYGGVSGLYDFGPVGCALKNNIIQTWRQHFIQEEQILEIDCTMLTPEPVLKTSGHVDKFANFMVKDVKNGECFRADHLLKAHLQKLMSDKKCSVEKKSEMESVLAQLDNYGQQELADLFVNYNVKSPITGNDLSPPVSFNLMFKTFIGPGGNMPGYLRPETAQGIFLNFKRLLEFNQGKLPFAAAQIGNSFRNEISPRSGLIRVREFTMAEIEHFVDPSEKDHPKFQNVADLHLYLYSAKAQVSGQSARKMRLGDAVEQGVINNTVLGYFIGRIYLYLTKVGISPDKLRFRQHMENEMAHYACDCWDAESKTSYGWIEIVGCADRSCYDLSCHARATKVPLVAEKPLKEPKTVNVVQFEPSKGAIGKAYKKDAKLVMEYLAICDECYITEMEMLLNEKGEFTIETEGKTFQLTKDMINVKRFQKTLYVEEVVPNVIEPSFGLGRIMYTVFEHTFHVREGDEQRTFFSFPAVVAPFKCSVLPLSQNQEFMPFVKELSEALTRHGVSHKVDDSSGSIGRRYARTDEIGVAFGVTIDFDTVNKTPHTATLRDRDSMRQIRAEISELPSIVQDLANGNITWADVEARYPLFEGQETGKKETIEE

SEQ ID NO: 15 - protein, 739 aa, Artificial Sequence
  GARS S211F
MPSPRPVLLRGARAALLLLLPPRLLARPSLLLRRSLSAASCPPISLPAAASRSSMDGAGAEEVLAPLRLAVRQQGDLVRKLKEDKAPQVDVDKAVAELKARKRVLEAKELALQPKDDIVDRAKMEDTLKRRFFYDQAFAIYGGVSGLYDFGPVGCALKNNIIQTWRQHFIQEEQILEIDCTMLTPEPVLKTSGHVDKFADFMVKDVKNGECFRADHLLKAHLQKLMSDKKCSVEKKSEMESVLAQLDNYGQQELADLFVNYNVKFPITGNDLSPPVSFNLMFKTFIGPGGNMPGYLRPETAQGIFLNFKRLLEFNQGKLPFAAAQIGNSFRNEISPRSGLIRVREFTMAEIEHFVDPSEKDHPKFQNVADLHLYLYSAKAQVSGQSARKMRLGDAVEQGVINNTVLGYFIGRIYLYLTKVGISPDKLRFRQHMENEMAHYACDCWDAESKTSYGWIEIVGCADRSCYDLSCHARATKVPLVAEKPLKEPKTVNVVQFEPSKGAIGKAYKKDAKLVMEYLAICDECYITEMEMLLNEKGEFTIETEGKTFQLTKDMINVKRFQKTLYVEEVVPNVIEPSFGLGRIMYTVFEHTFHVREGDEQRTFFSFPAVVAPFKCSVLPLSQNQEFMPFVKELSEALTRHGVSHKVDDSSGSIGRRYARTDEIGVAFGVTIDFDTVNKTPHTATLRDRDSMRQIRAEISELPSIVQDLANGNITWADVEARYPLFEGQETGKKETIEE

SEQ ID NO: 16 - protein, 739 aa, Artificial Sequence
  GARS L218Q
MPSPRPVLLRGARAALLLLLPPRLLARPSLLLRRSLSAASCPPISLPAAASRSSMDGAGAEEVLAPLRLAVRQQGDLVRKLKEDKAPQVDVDKAVAELKARKRVLEAKELALQPKDDIVDRAKMEDTLKRRFFYDQAFAIYGGVSGLYDFGPVGCALKNNIIQTWRQHFIQEEQILEIDCTMLTPEPVLKTSGHVDKFADFMVKDVKNGECFRADHLLKAHLQKLMSDKKCSVEKKSEMESVLAQLDNYGQQELADLFVNYNVKSPITGNDQSPPVSFNLMFKTFIGPGGNMPGYLRPETAQGIFLNFKRLLEFNQGKLPFAAAQIGNSFRNEISPRSGLIRVREFTMAEIEHFVDPSEKDHPKFQNVADLHLYLYSAKAQVSGQSARKMRLGDAVEQGVINNTVLGYFIGRIYLYLTKVGISPDKLRFRQHMENEMAHYACDCWDAESKTSYGWIEIVGCADRSCYDLSCHARATKVPLVAEKPLKEPKTVNVVQFEPSKGAIGKAYKKDAKLVMEYLAICDECYITEMEMLLNEKGEFTIETEGKTFQLTKDMINVKRFQKTLYVEEVVPNVIEPSFGLGRIMYTVFEHTFHVREGDEQRTFFSFPAVVAPFKCSVLPLSQNQEFMPFVKELSEALTRHGVSHKVDDSSGSIGRRYARTDEIGVAFGVTIDFDTVNKTPHTATLRDRDSMRQIRAEISELPSIVQDLANGNITWADVEARYPLFEGQETGKKETIEE

SEQ ID NO: 17 - protein, 739 aa, Artificial Sequence
  GARS G240R
MPSPRPVLLRGARAALLLLLPPRLLARPSLLLRRSLSAASCPPISLPAAASRSSMDGAGAEEVLAPLRLAVRQQGDLVRKLKEDKAPQVDVDKAVAELKARKRVLEAKELALQPKDDIVDRAKMEDTLKRRFFYDQAFAIYGGVSGLYDFGPVGCALKNNIIQTWRQHFIQEEQILEIDCTMLTPEPVLKTSGHVDKFADFMVKDVKNGECFRADHLLKAHLQKLMSDKKCSVEKKSEMESVLAQLDNYGQQELADLFVNYNVKSPITGNDLSPPVSFNLMFKTFIGPGGNMPRYLRPETAQGIFLNFKRLLEFNQGKLPFAAAQIGNSFRNEISPRSGLIRVREFTMAEIEHFVDPSEKDHPKFQNVADLHLYLYSAKAQVSGQSARKMRLGDAVEQGVINNTVLGYFIGRIYLYLTKVGISPDKLRFRQHMENEMAHYACDCWDAESKTSYGWIEIVGCADRSCYDLSCHARATKVPLVAEKPLKEPKTVNVVQFEPSKGAIGKAYKKDAKLVMEYLAICDECYITEMEMLLNEKGEFTIETEGKTFQLTKDMINVKRFQKTLYVEEVVPNVIEPSFGLGRIMYTVFEHTFHVREGDEQRTFFSFPAVVAPFKCSVLPLSQNQEFMPFVKELSEALTRHGVSHKVDDSSGSIGRRYARTDEIGVAFGVTIDFDTVNKTPHTATLRDRDSMRQIRAEISELPSIVQDLANGNITWADVEARYPLFEGQETGKKETIEE

SEQ ID NO: 18 - protein, 739 aa, Artificial Sequence
  GARS P244L
MPSPRPVLLRGARAALLLLLPPRLLARPSLLLRRSLSAASCPPISLPAAASRSSMDGAGAEEVLAPLRLAVRQQGDLVRKLKEDKAPQVDVDKAVAELKARKRVLEAKELALQPKDDIVDRAKMEDTLKRRFFYDQAFAIYGGVSGLYDFGPVGCALKNNIIQTWRQHFIQEEQILEIDCTMLTPEPVLKTSGHVDKFADFMVKDVKNGECFRADHLLKAHLQKLMSDKKCSVEKKSEMESVLAQLDNYGQQELADLFVNYNVKSPITGNDLSPPVSFNLMFKTFIGPGGNMPGYLRLETAQGIFLNFKRLLEFNQGKLPFAAAQIGNSFRNEISPRSGLIRVREFTMAEIEHFVDPSEKDHPKFQNVADLHLYLYSAKAQVSGQSARKMRLGDAVEQGVINNTVLGYFIGRIYLYLTKVGISPDKLRFRQHMENEMAHYACDCWDAESKTSYGWIEIVGCADRSCYDLSCHARATKVPLVAEKPLKEPKTVNVVQFEPSKGAIGKAYKKDAKLVMEYLAICDECYITEMEMLLNEKGEFTIETEGKTFQLTKDMINVKRFQKTLYVEEVVPNVIEPSFGLGRIMYTVFEHTFHVREGDEQRTFFSFPAVVAPFKCSVLPLSQNQEFMPFVKELSEALTRHGVSHKVDDSSGSIGRRYARTDEIGVAFGVTIDFDTVNKTPHTATLRDRDSMRQIRAEISELPSIVQDLANGNITWADVEARYPLFEGQETGKKETIEE

SEQ ID NO: 19 - protein, 739 aa, Artificial Sequence
  GARS E279D
MPSPRPVLLRGARAALLLLLPPRLLARPSLLLRRSLSAASCPPISLPAAASRSSMDGAGAEEVLAPLRLAVRQQGDLVRKLKEDKAPQVDVDKAVAELKARKRVLEAKELALQPKDDIVDRAKMEDTLKRRFFYDQAFAIYGGVSGLYDFGPVGCALKNNIIQTWRQHFIQEEQILEIDCTMLTPEPVLKTSGHVDKFADFMVKDVKNGECFRADHLLKAHLQKLMSDKKCSVEKKSEMESVLAQLDNYGQQELADLFVNYNVKSPITGNDLSPPVSFNLMFKTFIGPGGNMPGYLRPETAQGIFLNFKRLLEFNQGKLPFAAAQIGNSFRNDISPRSGLIRVREFTMAEIEHFVDPSEKDHPKFQNVADLHLYLYSAKAQVSGQSARKMRLGDAVEQGVINNTVLGYFIGRIYLYLTKVGISPDKLRFRQHMENEMAHYACDCWDAESKTSYGWIEIVGCADRSCYDLSCHARATKVPLVAEKPLKEPKTVNVVQFEPSKGAIGKAYKKDAKLVMEYLAICDECYITEMEMLLNEKGEFTIETEGKTFQLTKDMINVKRFQKTLYVEEVVPNVIEPSFGLGRIMYTVFEHTFHVREGDEQRTFFSFPAVVAPFKCSVLPLSQNQEFMPFVKELSEALTRHGVSHKVDDSSGSIGRRYARTDEIGVAFGVTIDFDTVNKTPHTATLRDRDSMRQIRAEISELPSIVQDLANGNITWADVEARYPLFEGQETGKKETIEE

SEQ ID NO: 20 - protein, 739 aa, Artificial Sequence
  GARS I280F
MPSPRPVLLRGARAALLLLLPPRLLARPSLLLRRSLSAASCPPISLPAAASRSSMDGAGAEEVLAPLRLAVRQQGDLVRKLKEDKAPQVDVDKAVAELKARKRVLEAKELALQPKDDIVDRAKMEDTLKRRFFYDQAFAIYGGVSGLYDFGPVGCALKNNIIQTWRQHFIQEEQILEIDCTMLTPEPVLKTSGHVDKFADFMVKDVKNGECFRADHLLKAHLQKLMSDKKCSVEKKSEMESVLAQLDNYGQQELADLFVNYNVKSPITGNDLSPPVSFNLMFKTFIGPGGNMPGYLRPETAQGIFLNFKRLLEFNQGKLPFAAAQIGNSFRNEFSPRSGLIRVREFTMAEIEHFVDPSEKDHPKFQNVADLHLYLYSAKAQVSGQSARKMRLGDAVEQGVINNTVLGYFIGRIYLYLTKVGISPDKLRFRQHMENEMAHYACDCWDAESKTSYGWIEIVGCADRSCYDLSCHARATKVPLVAEKPLKEPKTVNVVQFEPSKGAIGKAYKKDAKLVMEYLAICDECYITEMEMLLNEKGEFTIETEGKTFQLTKDMINVKRFQKTLYVEEVVPNVIEPSFGLGRIMYTVFEHTFHVREGDEQRTFFSFPAVVAPFKCSVLPLSQNQEFMPFVKELSEALTRHGVSHKVDDSSGSIGRRYARTDEIGVAFGVTIDFDTVNKTPHTATLRDRDSMRQIRAEISELPSIVQDLANGNITWADVEARYPLFEGQETGKKETIEE

SEQ ID NO: 21 - protein, 739 aa, Artificial Sequence
  GARS H418R
MPSPRPVLLRGARAALLLLLPPRLLARPSLLLRRSLSAASCPPISLPAAASRSSMDGAGAEEVLAPLRLAVRQQGDLVRKLKEDKAPQVDVDKAVAELKARKRVLEAKELALQPKDDIVDRAKMEDTLKRRFFYDQAFAIYGGVSGLYDFGPVGCALKNNIIQTWRQHFIQEEQILEIDCTMLTPEPVLKTSGHVDKFADFMVKDVKNGECFRADHLLKAHLQKLMSDKKCSVEKKSEMESVLAQLDNYGQQELADLFVNYNVKSPITGNDLSPPVSFNLMFKTFIGPGGNMPGYLRPETAQGIFLNFKRLLEFNQGKLPFAAAQIGNSFRNEISPRSGLIRVREFTMAEIEHFVDPSEKDHPKFQNVADLHLYLYSAKAQVSGQSARKMRLGDAVEQGVINNTVLGYFIGRIYLYLTKVGISPDKLRFRQHMENEMAHYACDCWDAESKTSYGWIEIVGCADRSCYDLSCRARATKVPLVAEKPLKEPKTVNVVQFEPSKGAIGKAYKKDAKLVMEYLAICDECYITEMEMLLNEKGEFTIETEGKTFQLTKDMINVKRFQKTLYVEEVVPNVIEPSFGLGRIMYTVFEHTFHVREGDEQRTFFSFPAVVAPFKCSVLPLSQNQEFMPFVKELSEALTRHGVSHKVDDSSGSIGRRYARTDEIGVAFGVTIDFDTVNKTPHTATLRDRDSMRQIRAEISELPSIVQDLANGNITWADVEARYPLFEGQETGKKETIEE

SEQ ID NO: 22 - protein, 739 aa, Artificial Sequence
  GARS D500N
MPSPRPVLLRGARAALLLLLPPRLLARPSLLLRRSLSAASCPPISLPAAASRSSMDGAGAEEVLAPLRLAVRQQGDLVRKLKEDKAPQVDVDKAVAELKARKRVLEAKELALQPKDDIVDRAKMEDTLKRRFFYDQAFAIYGGVSGLYDFGPVGCALKNNIIQTWRQHFIQEEQILEIDCTMLTPEPVLKTSGHVDKFADFMVKDVKNGECFRADHLLKAHLQKLMSDKKCSVEKKSEMESVLAQLDNYGQQELADLFVNYNVKSPITGNDLSPPVSFNLMFKTFIGPGGNMPGYLRPETAQGIFLNFKRLLEFNQGKLPFAAAQIGNSFRNEISPRSGLIRVREFTMAEIEHFVDPSEKDHPKFQNVADLHLYLYSAKAQVSGQSARKMRLGDAVEQGVINNTVLGYFIGRIYLYLTKVGISPDKLRFRQHMENEMAHYACDCWDAESKTSYGWIEIVGCADRSCYDLSCHARATKVPLVAEKPLKEPKTVNVVQFEPSKGAIGKAYKKDAKLVMEYLAICDECYITEMEMLLNEKGEFTIETEGKTFQLTKNMINVKRFQKTLYVEEVVPNVIEPSFGLGRIMYTVFEHTFHVREGDEQRTFFSFPAVVAPFKCSVLPLSQNQEFMPFVKELSEALTRHGVSHKVDDSSGSIGRRYARTDEIGVAFGVTIDFDTVNKTPHTATLRDRDSMRQIRAEISELPSIVQDLANGNITWADVEARYPLFEGQETGKKETIEE

SEQ ID NO: 23 - protein, 739 aa, Artificial Sequence
  GARS G526R
MPSPRPVLLRGARAALLLLLPPRLLARPSLLLRRSLSAASCPPISLPAAASRSSMDGAGAEEVLAPLRLAVRQQGDLVRKLKEDKAPQVDVDKAVAELKARKRVLEAKELALQPKDDIVDRAKMEDTLKRRFFYDQAFAIYGGVSGLYDFGPVGCALKNNIIQTWRQHFIQEEQILEIDCTMLTPEPVLKTSGHVDKFADFMVKDVKNGECFRADHLLKAHLQKLMSDKKCSVEKKSEMESVLAQLDNYGQQELADLFVNYNVKSPITGNDLSPPVSFNLMFKTFIGPGGNMPGYLRPETAQGIFLNFKRLLEFNQGKLPFAAAQIGNSFRNEISPRSGLIRVREFTMAEIEHFVDPSEKDHPKFQNVADLHLYLYSAKAQVSGQSARKMRLGDAVEQGVINNTVLGYFIGRIYLYLTKVGISPDKLRFRQHMENEMAHYACDCWDAESKTSYGWIEIVGCADRSCYDLSCHARATKVPLVAEKPLKEPKTVNVVQFEPSKGAIGKAYKKDAKLVMEYLAICDECYITEMEMLLNEKGEFTIETEGKTFQLTKDMINVKRFQKTLYVEEVVPNVIEPSFRLGRIMYTVFEHTFHVREGDEQRTFFSFPAVVAPFKCSVLPLSQNQEFMPFVKELSEALTRHGVSHKVDDSSGSIGRRYARTDEIGVAFGVTIDFDTVNKTPHTATLRDRDSMRQIRAEISELPSIVQDLANGNITWADVEARYPLFEGQETGKKETIEE

SEQ ID NO: 24 - protein, 739 aa, Artificial Sequence
  GARS S581L
MPSPRPVLLRGARAALLLLLPPRLLARPSLLLRRSLSAASCPPISLPAAASRSSMDGAGAEEVLAPLRLAVRQQGDLVRKLKEDKAPQVDVDKAVAELKARKRVLEAKELALQPKDDIVDRAKMEDTLKRRFFYDQAFAIYGGVSGLYDFGPVGCALKNNIIQTWRQHFIQEEQILEIDCTMLTPEPVLKTSGHVDKFADFMVKDVKNGECFRADHLLKAHLQKLMSDKKCSVEKKSEMESVLAQLDNYGQQELADLFVNYNVKSPITGNDLSPPVSFNLMFKTFIGPGGNMPGYLRPETAQGIFLNFKRLLEFNQGKLPFAAAQIGNSFRNEISPRSGLIRVREFTMAEIEHFVDPSEKDHPKFQNVADLHLYLYSAKAQVSGQSARKMRLGDAVEQGVINNTVLGYFIGRIYLYLTKVGISPDKLRFRQHMENEMAHYACDCWDAESKTSYGWIEIVGCADRSCYDLSCHARATKVPLVAEKPLKEPKTVNVVQFEPSKGAIGKAYKKDAKLVMEYLAICDECYITEMEMLLNEKGEFTIETEGKTFQLTKDMINVKRFQKTLYVEEVVPNVIEPSFGLGRIMYTVFEHTFHVREGDEQRTFFSFPAVVAPFKCSVLPLSQNQEFMPFVKELLEALTRHGVSHKVDDSSGSIGRRYARTDEIGVAFGVTIDFDTVNKTPHTATLRDRDSMRQIRAEISELPSIVQDLANGNITWADVEARYPLFEGQETGKKETIEE

SEQ ID NO: 25 - protein, 739 aa, Artificial Sequence
  GARS G598A
MPSPRPVLLRGARAALLLLLPPRLLARPSLLLRRSLSAASCPPISLPAAASRSSMDGAGAEEVLAPLRLAVRQQGDLVRKLKEDKAPQVDVDKAVAELKARKRVLEAKELALQPKDDIVDRAKMEDTLKRRFFYDQAFAIYGGVSGLYDFGPVGCALKNNIIQTWRQHFIQEEQILEIDCTMLTPEPVLKTSGHVDKFADFMVKDVKNGECFRADHLLKAHLQKLMSDKKCSVEKKSEMESVLAQLDNYGQQELADLFVNYNVKSPITGNDLSPPVSFNLMFKTFIGPGGNMPGYLRPETAQGIFLNFKRLLEFNQGKLPFAAAQIGNSFRNEISPRSGLIRVREFTMAEIEHFVDPSEKDHPKFQNVADLHLYLYSAKAQVSGQSARKMRLGDAVEQGVINNTVLGYFIGRIYLYLTKVGISPDKLRFRQHMENEMAHYACDCWDAESKTSYGWIEIVGCADRSCYDLSCHARATKVPLVAEKPLKEPKTVNVVQFEPSKGAIGKAYKKDAKLVMEYLAICDECYITEMEMLLNEKGEFTIETEGKTFQLTKDMINVKRFQKTLYVEEVVPNVIEPSFGLGRIMYTVFEHTFHVREGDEQRTFFSFPAVVAPFKCSVLPLSQNQEFMPFVKELSEALTRHGVSHKVDDSSASIGRRYARTDEIGVAFGVTIDFDTVNKTPHTATLRDRDSMRQIRAEISELPSIVQDLANGNITWADVEARYPLFEGQETGKKETIEE

SEQ ID NO: 26 - protein, 528 aa, Artificial Sequence
  YARS  WT
MGDAPSPEEKLHLITRNLQEVLGEEKLKEILKERELKIYWGTATTGKPHVAYFVPMSKIADFLKAGCEVTILFADLHAYLDNMKAPWELLELRVSYYENVIKAMLESIGVPLEKLKFIKGTDYQLSKEYTLDVYRLSSVVTQHDSKKAGAEVVKQVEHPLLSGLLYPGLQALDEEYLKVDAQFGGIDQRKIFTFAEKYLPALGYSKRVHLMNPMVPGLTGSKMSSSEEESKIDLLDRKEDVKKKLKKAFCEPGNVENNGVLSFIKHVLFPLKSEFVILRDEKWGGNKTYTAYVDLEKDFAAEVVHPGDLKNSVEVALNKLLDPIREKFNTPALKKLASAAYPDPSKQKPMAKGPAKNSEPEEVIPSRLDIRVGKIITVEKHPDADSLYVEKIDVGEAEPRTVVSGLVQFVPKEELQDRLVVVLCNLKPQKMRGVESQGMLLCASIEGINRQVEPLDPPAGSAPGEHVFVKGYEKGQPDEELKPKKKVFEKLQADFKISEECIAQWKQTNFMTKLGSISCKSLKGGNIS

SEQ ID NO: 27 - protein, 528 aa, Artificial Sequence
  YARS G41R
MGDAPSPEEKLHLITRNLQEVLGEEKLKEILKERELKIYWRTATTGKPHVAYFVPMSKIADFLKAGCEVTILFADLHAYLDNMKAPWELLELRVSYYENVIKAMLESIGVPLEKLKFIKGTDYQLSKEYTLDVYRLSSVVTQHDSKKAGAEVVKQVEHPLLSGLLYPGLQALDEEYLKVDAQFGGIDQRKIFTFAEKYLPALGYSKRVHLMNPMVPGLTGSKMSSSEEESKIDLLDRKEDVKKKLKKAFCEPGNVENNGVLSFIKHVLFPLKSEFVILRDEKWGGNKTYTAYVDLEKDFAAEVVHPGDLKNSVEVALNKLLDPIREKFNTPALKKLASAAYPDPSKQKPMAKGPAKNSEPEEVIPSRLDIRVGKIITVEKHPDADSLYVEKIDVGEAEPRTVVSGLVQFVPKEELQDRLVVVLCNLKPQKMRGVESQGMLLCASIEGINRQVEPLDPPAGSAPGEHVFVKGYEKGQPDEELKPKKKVFEKLQADFKISEECIAQWKQTNFMTKLGSISCKSLKGGNIS

SEQ ID NO: 28 - protein, 528 aa, Artificial Sequence
  YARS D81I
MGDAPSPEEKLHLITRNLQEVLGEEKLKEILKERELKIYWGTATTGKPHVAYFVPMSKIADFLKAGCEVTILFADLHAYLINMKAPWELLELRVSYYENVIKAMLESIGVPLEKLKFIKGTDYQLSKEYTLDVYRLSSVVTQHDSKKAGAEVVKQVEHPLLSGLLYPGLQALDEEYLKVDAQFGGIDQRKIFTFAEKYLPALGYSKRVHLMNPMVPGLTGSKMSSSEEESKIDLLDRKEDVKKKLKKAFCEPGNVENNGVLSFIKHVLFPLKSEFVILRDEKWGGNKTYTAYVDLEKDFAAEVVHPGDLKNSVEVALNKLLDPIREKFNTPALKKLASAAYPDPSKQKPMAKGPAKNSEPEEVIPSRLDIRVGKIITVEKHPDADSLYVEKIDVGEAEPRTVVSGLVQFVPKEELQDRLVVVLCNLKPQKMRGVESQGMLLCASIEGINRQVEPLDPPAGSAPGEHVFVKGYEKGQPDEELKPKKKVFEKLQADFKISEECIAQWKQTNFMTKLGSISCKSLKGGNIS

SEQ ID NO: 29 - protein, 524 aa, Artificial Sequence
  YARS Del153-156
MGDAPSPEEKLHLITRNLQEVLGEEKLKEILKERELKIYWGTATTGKPHVAYFVPMSKIADFLKAGCEVTILFADLHAYLDNMKAPWELLELRVSYYENVIKAMLESIGVPLEKLKFIKGTDYQLSKEYTLDVYRLSSVVTQHDSKKAGAEVEHPLLSGLLYPGLQALDEEYLKVDAQFGGIDQRKIFTFAEKYLPALGYSKRVHLMNPMVPGLTGSKMSSSEEESKIDLLDRKEDVKKKLKKAFCEPGNVENNGVLSFIKHVLFPLKSEFVILRDEKWGGNKTYTAYVDLEKDFAAEVVHPGDLKNSVEVALNKLLDPIREKFNTPALKKLASAAYPDPSKQKPMAKGPAKNSEPEEVIPSRLDIRVGKIITVEKHPDADSLYVEKIDVGEAEPRTVVSGLVQFVPKEELQDRLVVVLCNLKPQKMRGVESQGMLLCASIEGINRQVEPLDPPAGSAPGEHVFVKGYEKGQPDEELKPKKKVFEKLQADFKISEECIAQWKQTNFMTKLGSISCKSLKGGNIS

SEQ ID NO: 30 - protein, 528 aa, Artificial Sequence
  YARS E196K
MGDAPSPEEKLHLITRNLQEVLGEEKLKEILKERELKIYWGTATTGKPHVAYFVPMSKIADFLKAGCEVTILFADLHAYLDNMKAPWELLELRVSYYENVIKAMLESIGVPLEKLKFIKGTDYQLSKEYTLDVYRLSSVVTQHDSKKAGAEVVKQVEHPLLSGLLYPGLQALDEEYLKVDAQFGGIDQRKIFTFAKKYLPALGYSKRVHLMNPMVPGLTGSKMSSSEEESKIDLLDRKEDVKKKLKKAFCEPGNVENNGVLSFIKHVLFPLKSEFVILRDEKWGGNKTYTAYVDLEKDFAAEVVHPGDLKNSVEVALNKLLDPIREKFNTPALKKLASAAYPDPSKQKPMAKGPAKNSEPEEVIPSRLDIRVGKIITVEKHPDADSLYVEKIDVGEAEPRTVVSGLVQFVPKEELQDRLVVVLCNLKPQKMRGVESQGMLLCASIEGINRQVEPLDPPAGSAPGEHVFVKGYEKGQPDEELKPKKKVFEKLQADFKISEECIAQWKQTNFMTKLGSISCKSLKGGNIS

SEQ ID NO: 31 - protein, 968 aa, Homo sapiens
MDSTLTASEIRQRFIDFFKRNEHTYVHSSATIPLDDPTLLFANAGMNQFKPIFLNTIDPSHPMAKLSRAANTQKCIRAGGKHNDLDDVGKDVYHHTFFEMLGSWSFGDYFKELACKMALELLTQEFGIPIERLYVTYFGGDEAAGLEADLECKQIWQNLGLDDTKILPGNMKDNFWEMGDTGPCGPCSEIHYDRIGGRDAAHLVNQDDPNVLEIWNLVFIQYNREADGILKPLPKKSIDTGMGLERLVSVLQNKMSNYDTDLFVPYFEAIQKGTGARPYTGKVGAEDADGIDMAYRVLADHARTITVALADGGRPDNTGRGYVLRRILRRAVRYAHEKLNASRGFFATLVDVVVQSLGDAFPELKKDPDMVKDIINEEEVQFLKTLSRGRRILDRKIQSLGDSKTIPGDTAWLLYDTYGFPVDLTGLIAEEKGLVVDMDGFEEERKLAQLKSQGKGAGGEDLIMLDIYAIEELRARGLEVTDDSPKYNYHLDSSGSYVFENTVATVMALRREKMFVEEVSTGQECGVVLDKTCFYAEQGGQIYDEGYLVKVDDSSEDKTEFTVKNAQVRGGYVLHIGTIYGDLKVGDQVWLFIDEPRRRPIMSNHTATHILNFALRSVLGEADQKGSLVAPDRLRFDFTAKGAMSTQQIKKAEEIANEMIEAAKAVYTQDCPLAAAKAIQGLRAVFDETYPDPVRVVSIGVPVSELLDDPSGPAGSLTSVEFCGGTHLRNSSHAGAFVIVTEEAIAKGIRRIVAVTGAEAQKALRKAESLKKCLSVMEAKVKAQTAPNKDVQREIADLGEALATAVIPQWQKDELRETLKSLKKVMDDLDRASKADVQKRVLEKTKQFIDSNPNQPLVILEMESGASAKALNEALKLFKMHSPQTSAMLFTVDNEAGKITCLCQVPQNAANRGLKASEWVQQVSGLMDGKGGGKDVSAQATGKNVGCLQEALQLATSFAQLRLGDVKN

SEQ ID NO: 32 - protein, 968 aa, Artificial Sequence
  AARS N71Y
MDSTLTASEIRQRFIDFFKRNEHTYVHSSATIPLDDPTLLFANAGMNQFKPIFLNTIDPSHPMAKLSRAAYTQKCIRAGGKHNDLDDVGKDVYHHTFFEMLGSWSFGDYFKELACKMALELLTQEFGIPIERLYVTYFGGDEAAGLEADLECKQIWQNLGLDDTKILPGNMKDNFWEMGDTGPCGPCSEIHYDRIGGRDAAHLVNQDDPNVLEIWNLVFIQYNREADGILKPLPKKSIDTGMGLERLVSVLQNKMSNYDTDLFVPYFEAIQKGTGARPYTGKVGAEDADGIDMAYRVLADHARTITVALADGGRPDNTGRGYVLRRILRRAVRYAHEKLNASRGFFATLVDVVVQSLGDAFPELKKDPDMVKDIINEEEVQFLKTLSRGRRILDRKIQSLGDSKTIPGDTAWLLYDTYGFPVDLTGLIAEEKGLVVDMDGFEEERKLAQLKSQGKGAGGEDLIMLDIYAIEELRARGLEVTDDSPKYNYHLDSSGSYVFENTVATVMALRREKMFVEEVSTGQECGVVLDKTCFYAEQGGQIYDEGYLVKVDDSSEDKTEFTVKNAQVRGGYVLHIGTIYGDLKVGDQVWLFIDEPRRRPIMSNHTATHILNFALRSVLGEADQKGSLVAPDRLRFDFTAKGAMSTQQIKKAEEIANEMIEAAKAVYTQDCPLAAAKAIQGLRAVFDETYPDPVRVVSIGVPVSELLDDPSGPAGSLTSVEFCGGTHLRNSSHAGAFVIVTEEAIAKGIRRIVAVTGAEAQKALRKAESLKKCLSVMEAKVKAQTAPNKDVQREIADLGEALATAVIPQWQKDELRETLKSLKKVMDDLDRASKADVQKRVLEKTKQFIDSNPNQPLVILEMESGASAKALNEALKLFKMHSPQTSAMLFTVDNEAGKITCLCQVPQNAANRGLKASEWVQQVSGLMDGKGGGKDVSAQATGKNVGCLQEALQLATSFAQLRLGDVKN

SEQ ID NO: 33 - protein, 968 aa, Artificial Sequence
  AARS G102R
MDSTLTASEIRQRFIDFFKRNEHTYVHSSATIPLDDPTLLFANAGMNQFKPIFLNTIDPSHPMAKLSRAANTQKCIRAGGKHNDLDDVGKDVYHHTFFEMLRSWSFGDYFKELACKMALELLTQEFGIPIERLYVTYFGGDEAAGLEADLECKQIWQNLGLDDTKILPGNMKDNFWEMGDTGPCGPCSEIHYDRIGGRDAAHLVNQDDPNVLEIWNLVFIQYNREADGILKPLPKKSIDTGMGLERLVSVLQNKMSNYDTDLFVPYFEAIQKGTGARPYTGKVGAEDADGIDMAYRVLADHARTITVALADGGRPDNTGRGYVLRRILRRAVRYAHEKLNASRGFFATLVDVVVQSLGDAFPELKKDPDMVKDIINEEEVQFLKTLSRGRRILDRKIQSLGDSKTIPGDTAWLLYDTYGFPVDLTGLIAEEKGLVVDMDGFEEERKLAQLKSQGKGAGGEDLIMLDIYAIEELRARGLEVTDDSPKYNYHLDSSGSYVFENTVATVMALRREKMFVEEVSTGQECGVVLDKTCFYAEQGGQIYDEGYLVKVDDSSEDKTEFTVKNAQVRGGYVLHIGTIYGDLKVGDQVWLFIDEPRRRPIMSNHTATHILNFALRSVLGEADQKGSLVAPDRLRFDFTAKGAMSTQQIKKAEEIANEMIEAAKAVYTQDCPLAAAKAIQGLRAVFDETYPDPVRVVSIGVPVSELLDDPSGPAGSLTSVEFCGGTHLRNSSHAGAFVIVTEEAIAKGIRRIVAVTGAEAQKALRKAESLKKCLSVMEAKVKAQTAPNKDVQREIADLGEALATAVIPQWQKDELRETLKSLKKVMDDLDRASKADVQKRVLEKTKQFIDSNPNQPLVILEMESGASAKALNEALKLFKMHSPQTSAMLFTVDNEAGKITCLCQVPQNAANRGLKASEWVQQVSGLMDGKGGGKDVSAQATGKNVGCLQEALQLATSFAQLRLGDVKN

SEQ ID NO: 34 - protein, 968 aa, Artificial Sequence
  AARS R329H
MDSTLTASEIRQRFIDFFKRNEHTYVHSSATIPLDDPTLLFANAGMNQFKPIFLNTIDPSHPMAKLSRAANTQKCIRAGGKHNDLDDVGKDVYHHTFFEMLGSWSFGDYFKELACKMALELLTQEFGIPIERLYVTYFGGDEAAGLEADLECKQIWQNLGLDDTKILPGNMKDNFWEMGDTGPCGPCSEIHYDRIGGRDAAHLVNQDDPNVLEIWNLVFIQYNREADGILKPLPKKSIDTGMGLERLVSVLQNKMSNYDTDLFVPYFEAIQKGTGARPYTGKVGAEDADGIDMAYRVLADHARTITVALADGGRPDNTGRGYVLRRILHRAVRYAHEKLNASRGFFATLVDVVVQSLGDAFPELKKDPDMVKDIINEEEVQFLKTLSRGRRILDRKIQSLGDSKTIPGDTAWLLYDTYGFPVDLTGLIAEEKGLVVDMDGFEEERKLAQLKSQGKGAGGEDLIMLDIYAIEELRARGLEVTDDSPKYNYHLDSSGSYVFENTVATVMALRREKMFVEEVSTGQECGVVLDKTCFYAEQGGQIYDEGYLVKVDDSSEDKTEFTVKNAQVRGGYVLHIGTIYGDLKVGDQVWLFIDEPRRRPIMSNHTATHILNFALRSVLGEADQKGSLVAPDRLRFDFTAKGAMSTQQIKKAEEIANEMIEAAKAVYTQDCPLAAAKAIQGLRAVFDETYPDPVRVVSIGVPVSELLDDPSGPAGSLTSVEFCGGTHLRNSSHAGAFVIVTEEAIAKGIRRIVAVTGAEAQKALRKAESLKKCLSVMEAKVKAQTAPNKDVQREIADLGEALATAVIPQWQKDELRETLKSLKKVMDDLDRASKADVQKRVLEKTKQFIDSNPNQPLVILEMESGASAKALNEALKLFKMHSPQTSAMLFTVDNEAGKITCLCQVPQNAANRGLKASEWVQQVSGLMDGKGGGKDVSAQATGKNVGCLQEALQLATSFAQLRLGDVKN

SEQ ID NO: 35 - protein, 968 aa, Artificial Sequence
  AARS E688G
MDSTLTASEIRQRFIDFFKRNEHTYVHSSATIPLDDPTLLFANAGMNQFKPIFLNTIDPSHPMAKLSRAANTQKCIRAGGKHNDLDDVGKDVYHHTFFEMLGSWSFGDYFKELACKMALELLTQEFGIPIERLYVTYFGGDEAAGLEADLECKQIWQNLGLDDTKILPGNMKDNFWEMGDTGPCGPCSEIHYDRIGGRDAAHLVNQDDPNVLEIWNLVFIQYNREADGILKPLPKKSIDTGMGLERLVSVLQNKMSNYDTDLFVPYFEAIQKGTGARPYTGKVGAEDADGIDMAYRVLADHARTITVALADGGRPDNTGRGYVLRRILRRAVRYAHEKLNASRGFFATLVDVVVQSLGDAFPELKKDPDMVKDIINEEEVQFLKTLSRGRRILDRKIQSLGDSKTIPGDTAWLLYDTYGFPVDLTGLIAEEKGLVVDMDGFEEERKLAQLKSQGKGAGGEDLIMLDIYAIEELRARGLEVTDDSPKYNYHLDSSGSYVFENTVATVMALRREKMFVEEVSTGQECGVVLDKTCFYAEQGGQIYDEGYLVKVDDSSEDKTEFTVKNAQVRGGYVLHIGTIYGDLKVGDQVWLFIDEPRRRPIMSNHTATHILNFALRSVLGEADQKGSLVAPDRLRFDFTAKGAMSTQQIKKAEEIANEMIEAAKAVYTQDCPLAAAKAIQGLRAVFDGTYPDPVRVVSIGVPVSELLDDPSGPAGSLTSVEFCGGTHLRNSSHAGAFVIVTEEAIAKGIRRIVAVTGAEAQKALRKAESLKKCLSVMEAKVKAQTAPNKDVQREIADLGEALATAVIPQWQKDELRETLKSLKKVMDDLDRASKADVQKRVLEKTKQFIDSNPNQPLVILEMESGASAKALNEALKLFKMHSPQTSAMLFTVDNEAGKITCLCQVPQNAANRGLKASEWVQQVSGLMDGKGGGKDVSAQATGKNVGCLQEALQLATSFAQLRLGDVKN

SEQ ID NO: 36 - protein, 968 aa, Artificial Sequence
  AARS E778A
MDSTLTASEIRQRFIDFFKRNEHTYVHSSATIPLDDPTLLFANAGMNQFKPIFLNTIDPSHPMAKLSRAANTQKCIRAGGKHNDLDDVGKDVYHHTFFEMLGSWSFGDYFKELACKMALELLTQEFGIPIERLYVTYFGGDEAAGLEADLECKQIWQNLGLDDTKILPGNMKDNFWEMGDTGPCGPCSEIHYDRIGGRDAAHLVNQDDPNVLEIWNLVFIQYNREADGILKPLPKKSIDTGMGLERLVSVLQNKMSNYDTDLFVPYFEAIQKGTGARPYTGKVGAEDADGIDMAYRVLADHARTITVALADGGRPDNTGRGYVLRRILRRAVRYAHEKLNASRGFFATLVDVVVQSLGDAFPELKKDPDMVKDIINEEEVQFLKTLSRGRRILDRKIQSLGDSKTIPGDTAWLLYDTYGFPVDLTGLIAEEKGLVVDMDGFEEERKLAQLKSQGKGAGGEDLIMLDIYAIEELRARGLEVTDDSPKYNYHLDSSGSYVFENTVATVMALRREKMFVEEVSTGQECGVVLDKTCFYAEQGGQIYDEGYLVKVDDSSEDKTEFTVKNAQVRGGYVLHIGTIYGDLKVGDQVWLFIDEPRRRPIMSNHTATHILNFALRSVLGEADQKGSLVAPDRLRFDFTAKGAMSTQQIKKAEEIANEMIEAAKAVYTQDCPLAAAKAIQGLRAVFDETYPDPVRVVSIGVPVSELLDDPSGPAGSLTSVEFCGGTHLRNSSHAGAFVIVTEEAIAKGIRRIVAVTGAEAQKALRKAESLKKCLSVMAAKVKAQTAPNKDVQREIADLGEALATAVIPQWQKDELRETLKSLKKVMDDLDRASKADVQKRVLEKTKQFIDSNPNQPLVILEMESGASAKALNEALKLFKMHSPQTSAMLFTVDNEAGKITCLCQVPQNAANRGLKASEWVQQVSGLMDGKGGGKDVSAQATGKNVGCLQEALQLATSFAQLRLGDVKN

SEQ ID NO: 37 - protein, 968 aa, Artificial Sequence
  AARS D893N
MDSTLTASEIRQRFIDFFKRNEHTYVHSSATIPLDDPTLLFANAGMNQFKPIFLNTIDPSHPMAKLSRAANTQKCIRAGGKHNDLDDVGKDVYHHTFFEMLGSWSFGDYFKELACKMALELLTQEFGIPIERLYVTYFGGDEAAGLEADLECKQIWQNLGLDDTKILPGNMKDNFWEMGDTGPCGPCSEIHYDRIGGRDAAHLVNQDDPNVLEIWNLVFIQYNREADGILKPLPKKSIDTGMGLERLVSVLQNKMSNYDTDLFVPYFEAIQKGTGARPYTGKVGAEDADGIDMAYRVLADHARTITVALADGGRPDNTGRGYVLRRILRRAVRYAHEKLNASRGFFATLVDVVVQSLGDAFPELKKDPDMVKDIINEEEVQFLKTLSRGRRILDRKIQSLGDSKTIPGDTAWLLYDTYGFPVDLTGLIAEEKGLVVDMDGFEEERKLAQLKSQGKGAGGEDLIMLDIYAIEELRARGLEVTDDSPKYNYHLDSSGSYVFENTVATVMALRREKMFVEEVSTGQECGVVLDKTCFYAEQGGQIYDEGYLVKVDDSSEDKTEFTVKNAQVRGGYVLHIGTIYGDLKVGDQVWLFIDEPRRRPIMSNHTATHILNFALRSVLGEADQKGSLVAPDRLRFDFTAKGAMSTQQIKKAEEIANEMIEAAKAVYTQDCPLAAAKAIQGLRAVFDETYPDPVRVVSIGVPVSELLDDPSGPAGSLTSVEFCGGTHLRNSSHAGAFVIVTEEAIAKGIRRIVAVTGAEAQKALRKAESLKKCLSVMEAKVKAQTAPNKDVQREIADLGEALATAVIPQWQKDELRETLKSLKKVMDDLDRASKADVQKRVLEKTKQFIDSNPNQPLVILEMESGASAKALNEALKLFKMHSPQTSAMLFTVNNEAGKITCLCQVPQNAANRGLKASEWVQQVSGLMDGKGGGKDVSAQATGKNVGCLQEALQLATSFAQLRLGDVKN

SEQ ID NO: 38 - protein, 469 aa, Homo sapiens
MAERAALEELVKLQGERVRGLKQQKASAELIEEEVAKLLKLKAQLGPDESKQKFVLKTPKETLMGKYGEDSKLIYDLKDQGGELLSLRYDLTVPFARYLAMNKLTNIKRYHIAKVYRRDNPAMTRGRYREFYQCDFDIAGNFDPMIPDAECLKIMCEILSSLQIGDFLVKVNDRRILDGMFAICGVSDSKFRTICSSVDKLDKVSWEEVKNEMVGEKGLAPEVADRIGDYVQQHGGVSLVEQLLQDPKLSQNKQALEGLGDLKLLFEYLTLFGIDDKISFDLSLARGLDYYTGVIYEAVLLQTPAQAGEEPLGVGSVAAGGRYDGLVGMFDPKGRKVPCVGLSIGVERIFSIVEQRLEALEEKIRTTETQVLVASAQKKLLEERLKLVSELWDAGIKAELLYKKNPKLLNQLQYCEEAGIPLVAIIGEQELKDGVIKLRSVTSREEVDVRREDLVEEIKRRTGQPLCIC

SEQ ID NO: 39 - protein, 469 aa, Artificial Sequence
  HARS T132I
MAERAALEELVKLQGERVRGLKQQKASAELIEEEVAKLLKLKAQLGPDESKQKFVLKTPKETLMGKYGEDSKLIYDLKDQGGELLSLRYDLIVPFARYLAMNKLTNIKRYHIAKVYRRDNPAMTRGRYREFYQCDFDIAGNFDPMIPDAECLKIMCEILSSLQIGDFLVKVNDRRILDGMFAICGVSDSKFRTICSSVDKLDKVSWEEVKNEMVGEKGLAPEVADRIGDYVQQHGGVSLVEQLLQDPKLSQNKQALEGLGDLKLLFEYLTLFGIDDKISFDLSLARGLDYYTGVIYEAVLLQTPAQAGEEPLGVGSVAAGGRYDGLVGMFDPKGRKVPCVGLSIGVERIFSIVEQRLEALEEKIRTTETQVLVASAQKKLLEERLKLVSELWDAGIKAELLYKKNPKLLNQLQYCEEAGIPLVAIIGEQELKDGVIKLRSVTSREEVDVRREDLVEEIKRRTGQPLCIC

SEQ ID NO: 40 - protein, 469 aa, Artificial Sequence
  HARS P134H
MAERAALEELVKLQGERVRGLKQQKASAELIEEEVAKLLKLKAQLGPDESKQKFVLKTPKETLMGKYGEDSKLIYDLKDQGGELLSLRYDLTVHFARYLAMNKLTNIKRYHIAKVYRRDNPAMTRGRYREFYQCDFDIAGNFDPMIPDAECLKIMCEILSSLQIGDFLVKVNDRRILDGMFAICGVSDSKFRTICSSVDKLDKVSWEEVKNEMVGEKGLAPEVADRIGDYVQQHGGVSLVEQLLQDPKLSQNKQALEGLGDLKLLFEYLTLFGIDDKISFDLSLARGLDYYTGVIYEAVLLQTPAQAGEEPLGVGSVAAGGRYDGLVGMFDPKGRKVPCVGLSIGVERIFSIVEQRLEALEEKIRTTETQVLVASAQKKLLEERLKLVSELWDAGIKAELLYKKNPKLLNQLQYCEEAGIPLVAIIGEQELKDGVIKLRSVTSREEVDVRREDLVEEIKRRTGQPLCIC

SEQ ID NO: 41 - protein, 469 aa, Artificial Sequence
  HARS R137Q
MAERAALEELVKLQGERVRGLKQQKASAELIEEEVAKLLKLKAQLGPDESKQKFVLKTPKETLMGKYGEDSKLIYDLKDQGGELLSLRYDLTVPFAQYLAMNKLTNIKRYHIAKVYRRDNPAMTRGRYREFYQCDFDIAGNFDPMIPDAECLKIMCEILSSLQIGDFLVKVNDRRILDGMFAICGVSDSKFRTICSSVDKLDKVSWEEVKNEMVGEKGLAPEVADRIGDYVQQHGGVSLVEQLLQDPKLSQNKQALEGLGDLKLLFEYLTLFGIDDKISFDLSLARGLDYYTGVIYEAVLLQTPAQAGEEPLGVGSVAAGGRYDGLVGMFDPKGRKVPCVGLSIGVERIFSIVEQRLEALEEKIRTTETQVLVASAQKKLLEERLKLVSELWDAGIKAELLYKKNPKLLNQLQYCEEAGIPLVAIIGEQELKDGVIKLRSVTSREEVDVRREDLVEEIKRRTGQPLCIC

SEQ ID NO: 42 - protein, 469 aa, Artificial Sequence
  HARS D175E
MAERAALEELVKLQGERVRGLKQQKASAELIEEEVAKLLKLKAQLGPDESKQKFVLKTPKETLMGKYGEDSKLIYDLKDQGGELLSLRYDLTVPFARYLAMNKLTNIKRYHIAKVYRRDNPAMTRGRYREFYQCEFDIAGNFDPMIPDAECLKIMCEILSSLQIGDFLVKVNDRRILDGMFAICGVSDSKFRTICSSVDKLDKVSWEEVKNEMVGEKGLAPEVADRIGDYVQQHGGVSLVEQLLQDPKLSQNKQALEGLGDLKLLFEYLTLFGIDDKISFDLSLARGLDYYTGVIYEAVLLQTPAQAGEEPLGVGSVAAGGRYDGLVGMFDPKGRKVPCVGLSIGVERIFSIVEQRLEALEEKIRTTETQVLVASAQKKLLEERLKLVSELWDAGIKAELLYKKNPKLLNQLQYCEEAGIPLVAIIGEQELKDGVIKLRSVTSREEVDVRREDLVEEIKRRTGQPLCIC

SEQ ID NO: 43 - protein, 469 aa, Artificial Sequence
  HARS D364Y
MAERAALEELVKLQGERVRGLKQQKASAELIEEEVAKLLKLKAQLGPDESKQKFVLKTPKETLMGKYGEDSKLIYDLKDQGGELLSLRYDLTVPFARYLAMNKLTNIKRYHIAKVYRRDNPAMTRGRYREFYQCDFDIAGNFDPMIPDAECLKIMCEILSSLQIGDFLVKVNDRRILDGMFAICGVSDSKFRTICSSVDKLDKVSWEEVKNEMVGEKGLAPEVADRIGDYVQQHGGVSLVEQLLQDPKLSQNKQALEGLGDLKLLFEYLTLFGIDDKISFDLSLARGLDYYTGVIYEAVLLQTPAQAGEEPLGVGSVAAGGRYYGLVGMFDPKGRKVPCVGLSIGVERIFSIVEQRLEALEEKIRTTETQVLVASAQKKLLEERLKLVSELWDAGIKAELLYKKNPKLLNQLQYCEEAGIPLVAIIGEQELKDGVIKLRSVTSREEVDVRREDLVEEIKRRTGQPLCIC

SEQ ID NO: 44 - protein, 625 aa, Homo sapiens
MLTQAAVRLVRGSLRKTSWAEWGHRELRLGQLAPFTAPHKDKSFSDQRSELKRRLKAEKKVAEKEAKQKELSEKQLSQATAAATNHTTDNGVGPEEESVDPNQYYKIRSQAIHQLKVNGEDPYPHKFHVDISLTDFIQKYSHLQPGDHLTDITLKVAGRIHAKRASGGKLIFYDLRGEGVKLQVMANSRNYKSEEEFIHINNKLRRGDIIGVQGNPGKTKKGELSIIPYEITLLSPCLHMLPHLHFGLKDKETRYRQRYLDLILNDFVRQKFIIRSKIITYIRSFLDELGFLEIETPMMNIIPGGAVAKPFITYHNELDMNLYMRIAPELYHKMLVVGGIDRVYEIGRQFRNEGIDLTHNPEFTTCEFYMAYADYHDLMEITEKMVSGMVKHITGSYKVTYHPDGPEGQAYDVDFTPPFRRINMVEELEKALGMKLPETNLFETEETRKILDDICVAKAVECPPPRTTARLLDKLVGEFLEVTCINPTFICDHPQIMSPLAKWHRSKEGLTERFELFVMKKEICNAYTELNDPMRQRQLFEEQAKAKAAGDDEAMFIDENFCTALEYGLPPTAGWGMGIDRVAMFLTDSNNIKEVLLFPAMKPEDKKENVATTDTLESTTVGTSV

SEQ ID NO: 45 - protein, 625 aa, Artificial Sequence
  KARS L133H
MLTQAAVRLVRGSLRKTSWAEWGHRELRLGQLAPFTAPHKDKSFSDQRSELKRRLKAEKKVAEKEAKQKELSEKQLSQATAAATNHTTDNGVGPEEESVDPNQYYKIRSQAIHQLKVNGEDPYPHKFHVDISHTDFIQKYSHLQPGDHLTDITLKVAGRIHAKRASGGKLIFYDLRGEGVKLQVMANSRNYKSEEEFIHINNKLRRGDIIGVQGNPGKTKKGELSIIPYEITLLSPCLHMLPHLHFGLKDKETRYRQRYLDLILNDFVRQKFIIRSKIITYIRSFLDELGFLEIETPMMNIIPGGAVAKPFITYHNELDMNLYMRIAPELYHKMLVVGGIDRVYEIGRQFRNEGIDLTHNPEFTTCEFYMAYADYHDLMEITEKMVSGMVKHITGSYKVTYHPDGPEGQAYDVDFTPPFRRINMVEELEKALGMKLPETNLFETEETRKILDDICVAKAVECPPPRTTARLLDKLVGEFLEVTCINPTFICDHPQIMSPLAKWHRSKEGLTERFELFVMKKEICNAYTELNDPMRQRQLFEEQAKAKAAGDDEAMFIDENFCTALEYGLPPTAGWGMGIDRVAMFLTDSNNIKEVLLFPAMKPEDKKENVATTDTLESTTVGTSV

SEQ ID NO: 46 - protein, 180 aa, Artificial Sequence
  KARS Y173SerfsX7
MLTQAAVRLVRGSLRKTSWAEWGHRELRLGQLAPFTAPHKDKSFSDQRSELKRRLKAEKKVAEKEAKQKELSEKQLSQATAAATNHTTDNGVGPEEESVDPNQYYKIRSQAIHQLKVNGEDPYPHKFHVDISLTDFIQKYSHLQPGDHLTDITLKVAGRIHAKRASGGKLIFYLIFEERG

SEQ ID NO: 47 - protein, 625 aa, Artificial Sequence
  KARS I302M
MLTQAAVRLVRGSLRKTSWAEWGHRELRLGQLAPFTAPHKDKSFSDQRSELKRRLKAEKKVAEKEAKQKELSEKQLSQATAAATNHTTDNGVGPEEESVDPNQYYKIRSQAIHQLKVNGEDPYPHKFHVDISLTDFIQKYSHLQPGDHLTDITLKVAGRIHAKRASGGKLIFYDLRGEGVKLQVMANSRNYKSEEEFIHINNKLRRGDIIGVQGNPGKTKKGELSIIPYEITLLSPCLHMLPHLHFGLKDKETRYRQRYLDLILNDFVRQKFIIRSKIITYIRSFLDELGFLEIETPMMNIMPGGAVAKPFITYHNELDMNLYMRIAPELYHKMLVVGGIDRVYEIGRQFRNEGIDLTHNPEFTTCEFYMAYADYHDLMEITEKMVSGMVKHITGSYKVTYHPDGPEGQAYDVDFTPPFRRINMVEELEKALGMKLPETNLFETEETRKILDDICVAKAVECPPPRTTARLLDKLVGEFLEVTCINPTFICDHPQIMSPLAKWHRSKEGLTERFELFVMKKEICNAYTELNDPMRQRQLFEEQAKAKAAGDDEAMFIDENFCTALEYGLPPTAGWGMGIDRVAMFLTDSNNIKEVLLFPAMKPEDKKENVATTDTLESTTVGTSV

SEQ ID NO: 48 - protein, 625 aa, Artificial Sequence [sequence omitted]

SEQ ID NO: 49 - protein, 900 aa, Homo sapiens
MRLFVSDGVPGCLPVLAAAGRARGRAEVLISTVGPEDCVVPFLTRPKVPVLQLDSGNYLFSTSAICRYFFLLSGWEQDDLTNQWLEWEATELQPALSAALYYLVVQGKKGEDVLGSVRRALTHIDHSLSRQNCPFLAGETESLADIVLWGALYPLLQDPAYLPEELSALHSWFQTLSTQEPCQRAAETVLKQQGVLALRPYLQKQPQPSPAEGRAVTNEPEEEELATLSEEEIAMAVTAWEKGLESLPPLRPQQNPVLPVAGERNVLITSALPYVNNVPHLGNIIGCVLSADVFARYSRLRQWNTLYLCGTDEYGTATETKALEEGLTPQEICDKYHIIHADIYRWFNISFDIFGRTTTPQQTKITQDIFQQLLKRGFVLQDTVEQLRCEHCARFLADRFVEGVCPFCGYEEARGDQCDKCGKLINAVELKKPQCKVCRSCPVVQSSQHLFLDLPKLEKRLEEWLGRTLPGSDWTPNAQFITRSWLRDGLKPRCITRDLKWGTPVPLEGFEDKVFYVWFDATIGYLSITANYTDQWERWWKNPEQVDLYQFMAKDNVPFHSLVFPCSALGAEDNYTLVSHLIATEYLNYEDGKFSKSRGVGVFGDMAQDTGIPADIWRFYLLYIRPEGQDSAFSWTDLLLKNNSELLNNLGNFINRAGMFVSKFFGGYVPEMVLTPDDQRLLAHVTLELQHYHQLLEKVRIRDALRSILTISRHGNQYIQVNEPWKRIKGSEADRQRAGTVTGLAVNIAALLSVMLQPYMPTVSATIQAQLQLPPPACSILLTNFLCTLPAGHQIGTVSPLFQKLENDQIESLRQRFGGGQAKTSPKPAVVETVTTAKPQQIQALMDEVTKQGNIVRELKAQKADKNEVAAEVAKLLDLKKQLAVAEGKPPEAPKGKKKK

SEQ ID NO: 50 - protein, 900 aa, Artificial Sequence
  MARS R618C
MRLFVSDGVPGCLPVLAAAGRARGRAEVLISTVGPEDCVVPFLTRPKVPVLQLDSGNYLFSTSAICRYFFLLSGWEQDDLTNQWLEWEATELQPALSAALYYLVVQGKKGEDVLGSVRRALTHIDHSLSRQNCPFLAGETESLADIVLWGALYPLLQDPAYLPEELSALHSWFQTLSTQEPCQRAAETVLKQQGVLALRPYLQKQPQPSPAEGRAVTNEPEEEELATLSEEEIAMAVTAWEKGLESLPPLRPQQNPVLPVAGERNVLITSALPYVNNVPHLGNIIGCVLSADVFARYSRLRQWNTLYLCGTDEYGTATETKALEEGLTPQEICDKYHIIHADIYRWFNISFDIFGRTTTPQQTKITQDIFQQLLKRGFVLQDTVEQLRCEHCARFLADRFVEGVCPFCGYEEARGDQCDKCGKLINAVELKKPQCKVCRSCPVVQSSQHLFLDLPKLEKRLEEWLGRTLPGSDWTPNAQFITRSWLRDGLKPRCITRDLKWGTPVPLEGFEDKVFYVWFDATIGYLSITANYTDQWERWWKNPEQVDLYQFMAKDNVPFHSLVFPCSALGAEDNYTLVSHLIATEYLNYEDGKFSKSRGVGVFGDMAQDTGIPADIWCFYLLYIRPEGQDSAFSWTDLLLKNNSELLNNLGNFINRAGMFVSKFFGGYVPEMVLTPDDQRLLAHVTLELQHYHQLLEKVRIRDALRSILTISRHGNQYIQVNEPWKRIKGSEADRQRAGTVTGLAVNIAALLSVMLQPYMPTVSATIQAQLQLPPPACSILLTNFLCTLPAGHQIGTVSPLFQKLENDQIESLRQRFGGGQAKTSPKPAVVETVTTAKPQQIQALMDEVTKQGNIVRELKAQKADKNEVAAEVAKLLDLKKQLAVAEGKPPEAPKGKKKK

SEQ ID NO: 51 - protein, 900 aa, Artificial Sequence
  MARS P800T
MRLFVSDGVPGCLPVLAAAGRARGRAEVLISTVGPEDCVVPFLTRPKVPVLQLDSGNYLFSTSAICRYFFLLSGWEQDDLTNQWLEWEATELQPALSAALYYLVVQGKKGEDVLGSVRRALTHIDHSLSRQNCPFLAGETESLADIVLWGALYPLLQDPAYLPEELSALHSWFQTLSTQEPCQRAAETVLKQQGVLALRPYLQKQPQPSPAEGRAVTNEPEEEELATLSEEEIAMAVTAWEKGLESLPPLRPQQNPVLPVAGERNVLITSALPYVNNVPHLGNIIGCVLSADVFARYSRLRQWNTLYLCGTDEYGTATETKALEEGLTPQEICDKYHIIHADIYRWFNISFDIFGRTTTPQQTKITQDIFQQLLKRGFVLQDTVEQLRCEHCARFLADRFVEGVCPFCGYEEARGDQCDKCGKLINAVELKKPQCKVCRSCPVVQSSQHLFLDLPKLEKRLEEWLGRTLPGSDWTPNAQFITRSWLRDGLKPRCITRDLKWGTPVPLEGFEDKVFYVWFDATIGYLSITANYTDQWERWWKNPEQVDLYQFMAKDNVPFHSLVFPCSALGAEDNYTLVSHLIATEYLNYEDGKFSKSRGVGVFGDMAQDTGIPADIWRFYLLYIRPEGQDSAFSWTDLLLKNNSELLNNLGNFINRAGMFVSKFFGGYVPEMVLTPDDQRLLAHVTLELQHYHQLLEKVRIRDALRSILTISRHGNQYIQVNEPWKRIKGSEADRQRAGTVTGLAVNIAALLSVMLQPYMPTVSATIQAQLQLPPPACSILLTNFLCTLPAGHQIGTVSTLFQKLENDQIESLRQRFGGGQAKTSPKPAVVETVTTAKPQQIQALMDEVTKQGNIVRELKAQKADKNEVAAEVAKLLDLKKQLAVAEGKPPEAPKGKKKK

SEQ ID NO: 52 - protein, 644 aa, Homo sapiens
MERGLPLLCAVLALVLAPAGAFRNDKCGDTIKIESPGYLTSPGYPHSYHPSEKCEWLIQAPDPYQRIMINFNPHFDLEDRDCKYDYVEVFDGENENGHFRGKFCGKIAPPPVVSSGPFLFIKFVSDYETHGAGFSIRYEIFKRGPECSQNYTTPSGVIKSPGFPEKYPNSLECTYIVFAPKMSEIILEFESFDLEPDSNPPGGMFCRYDRLEIWDGFPDVGPHIGRYCGQKTPGRIRSSSGILSMVFYTDSAIAKEGFSANYSVLQSSVSEDFKCMEALGMESGEIHSDQITASSQYSTNWSAERSRLNYPENGWTPGEDSYREWIQVDLGLLRFVTAVGTQGAISKETKKKYYVKTYKIDVSSNGEDWITIKEGNKPVLFQGNTNPTDVVVAVFPKPLITRFVRIKPATWETGISMRFEVYGCKITDYPCSGMLGMVSGLISDSQITSSNQGDRNWMPENIRLVTSRSGWALPPAPHSYINEWLQIDLGEEKIVRGIIIQGGKHRENKVFMRKFKIGYSNNGSDWKMIMDDSKRKAKSFEGNNNYDTPELRTFPALSTRFIRIYPERATHGGLGLRMELLGCEVEAPTAGPTTPNGNLVDECDDDQANCHSGTGDDFQLTGGTTVLATEKPTVIDSTIQSGIK

SEQ ID NO: 53 - protein, 158 aa, Homo sapiens
EDFKCMEALGMESGEIHSDQITASSQYSTNWSAERSRLNYPENGWTPGEDSYREWIQVDLGLLRFVTAVGTQGAISKETKKKYYVKTYKIDVSSNGEDWITIKEGNKPVLFQGNTNPTDVVVAVFPKPLITRFVRIKPATWETGISMRFEVYGCKITD

SEQ ID NO: 54 - protein, 412 aa, Homo sapiens
MTDRQTDTAPSPSYHLLPGRRRTVDAAASRGQGPEPAPGGGVEGVGARGVALKLFVQLLGCSRFGGAVVRAGEAEPSGAARSASSGREEPQPEEGEEEEEKEEERGPQWRLGARKPGSWTGEAAVCADSAPAARAPQALARASGRGGRVARRGAEESGPPHSPSRRGSASRAGPGRASETMNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRYKSWSVYVGARCCLMPWSLPGPHPCGPCSERRKHLFVQDPQTCKCSCKNTDSRCKARQLELNERTCRCDKPRR